SEQUENCE LISTING (351 sequences)

SEQ ID NO: 1 - DNA, 53 bp, Artificial Sequence
  synthetic
ggatgtgtctgcggcgttttatcatcttcctcttcatcctgctgctatgcctc

SEQ ID NO: 2 - DNA, 28 bp, Artificial Sequence
  synthetic
ggatgtgtctgcggcgttttatcatctt

SEQ ID NO: 3 - DNA, 28 bp, Artificial Sequence
  synthetic
gatgtgtctgcggcgttttatcatcttc

SEQ ID NO: 4 - DNA, 28 bp, Artificial Sequence
  synthetic
atgtgtctgcggcgttttatcatcttcc

SEQ ID NO: 5 - DNA, 28 bp, Artificial Sequence
  synthetic
tgtgtctgcggcgttttatcatcttcct

SEQ ID NO: 6 - DNA, 28 bp, Artificial Sequence
  synthetic
gtgtctgcggcgttttatcatcttcctc

SEQ ID NO: 7 - DNA, 28 bp, Artificial Sequence
  synthetic
tgtctgcggcgttttatcatcttcctct

SEQ ID NO: 8 - DNA, 28 bp, Artificial Sequence
  synthetic
gtctgcggcgttttatcatcttcctctt

SEQ ID NO: 9 - DNA, 28 bp, Artificial Sequence
  synthetic
tctgcggcgttttatcatcttcctcttc

SEQ ID NO: 10 - DNA, 28 bp, Artificial Sequence
  synthetic
ctgcggcgttttatcatcttcctcttca

SEQ ID NO: 11 - DNA, 28 bp, Artificial Sequence
  synthetic
tgcggcgttttatcatcttcctcttcat

SEQ ID NO: 12 - DNA, 28 bp, Artificial Sequence
  synthetic
gcggcgttttatcatcttcctcttcatc

SEQ ID NO: 13 - DNA, 28 bp, Artificial Sequence
  synthetic
cggcgttttatcatcttcctcttcatcc

SEQ ID NO: 14 - DNA, 28 bp, Artificial Sequence
  synthetic
ggcgttttatcatcttcctcttcatcct

SEQ ID NO: 15 - DNA, 28 bp, Artificial Sequence
  synthetic
gcgttttatcatcttcctcttcatcctg

SEQ ID NO: 16 - DNA, 28 bp, Artificial Sequence
  synthetic
cgttttatcatcttcctcttcatcctgc

SEQ ID NO: 17 - DNA, 28 bp, Artificial Sequence
  synthetic
gttttatcatcttcctcttcatcctgct

SEQ ID NO: 18 - DNA, 28 bp, Artificial Sequence
  synthetic
ttttatcatcttcctcttcatcctgctg

SEQ ID NO: 19 - DNA, 28 bp, Artificial Sequence
  synthetic
tttatcatcttcctcttcatcctgctgc

SEQ ID NO: 20 - DNA, 28 bp, Artificial Sequence
  synthetic
ttatcatcttcctcttcatcctgctgct

SEQ ID NO: 21 - DNA, 28 bp, Artificial Sequence
  synthetic
tatcatcttcctcttcatcctgctgcta

SEQ ID NO: 22 - DNA, 28 bp, Artificial Sequence
  synthetic
atcatcttcctcttcatcctgctgctat

SEQ ID NO: 23 - DNA, 28 bp, Artificial Sequence
  synthetic
tcatcttcctcttcatcctgctgctatg

SEQ ID NO: 24 - DNA, 28 bp, Artificial Sequence
  synthetic
catcttcctcttcatcctgctgctatgc

SEQ ID NO: 25 - DNA, 28 bp, Artificial Sequence
  synthetic
atcttcctcttcatcctgctgctatgcc

SEQ ID NO: 26 - DNA, 28 bp, Artificial Sequence
  synthetic
tcttcctcttcatcctgctgctatgcct

SEQ ID NO: 27 - DNA, 28 bp, Artificial Sequence
  synthetic
cttcctcttcatcctgctgctatgcctc

SEQ ID NO: 28 - DNA, 53 bp, Artificial Sequence
  synthetic
aaatttcatggggtatgtaattggatgttggggtacattaccacaggaacata

SEQ ID NO: 29 - DNA, 28 bp, Artificial Sequence
  synthetic
aaatttcatggggtatgtaattggatgt

SEQ ID NO: 30 - DNA, 28 bp, Artificial Sequence
  synthetic
aatttcatggggtatgtaattggatgtt

SEQ ID NO: 31 - DNA, 28 bp, Artificial Sequence
  synthetic
atttcatggggtatgtaattggatgttg

SEQ ID NO: 32 - DNA, 28 bp, Artificial Sequence
  synthetic
tttcatggggtatgtaattggatgttgg

SEQ ID NO: 33 - DNA, 28 bp, Artificial Sequence
  synthetic
ttcatggggtatgtaattggatgttggg

SEQ ID NO: 34 - DNA, 28 bp, Artificial Sequence
  synthetic
tcatggggtatgtaattggatgttgggg

SEQ ID NO: 35 - DNA, 28 bp, Artificial Sequence
  synthetic
catggggtatgtaattggatgttggggt

SEQ ID NO: 36 - DNA, 28 bp, Artificial Sequence
  synthetic
atggggtatgtaattggatgttggggta

SEQ ID NO: 37 - DNA, 28 bp, Artificial Sequence
  synthetic
tggggtatgtaattggatgttggggtac

SEQ ID NO: 38 - DNA, 28 bp, Artificial Sequence
  synthetic
ggggtatgtaattggatgttggggtaca

SEQ ID NO: 39 - DNA, 28 bp, Artificial Sequence
  synthetic
gggtatgtaattggatgttggggtacat

SEQ ID NO: 40 - DNA, 28 bp, Artificial Sequence
  synthetic
ggtatgtaattggatgttggggtacatt

SEQ ID NO: 41 - DNA, 28 bp, Artificial Sequence
  synthetic
gtatgtaattggatgttggggtacatta

SEQ ID NO: 42 - DNA, 28 bp, Artificial Sequence
  synthetic
tatgtaattggatgttggggtacattac

SEQ ID NO: 43 - DNA, 28 bp, Artificial Sequence
  synthetic
atgtaattggatgttggggtacattacc

SEQ ID NO: 44 - DNA, 28 bp, Artificial Sequence
  synthetic
tgtaattggatgttggggtacattacca

SEQ ID NO: 45 - DNA, 28 bp, Artificial Sequence
  synthetic
gtaattggatgttggggtacattaccac

SEQ ID NO: 46 - DNA, 28 bp, Artificial Sequence
  synthetic
taattggatgttggggtacattaccaca

SEQ ID NO: 47 - DNA, 28 bp, Artificial Sequence
  synthetic
aattggatgttggggtacattaccacag

SEQ ID NO: 48 - DNA, 28 bp, Artificial Sequence
  synthetic
attggatgttggggtacattaccacagg

SEQ ID NO: 49 - DNA, 28 bp, Artificial Sequence
  synthetic
ttggatgttggggtacattaccacagga

SEQ ID NO: 50 - DNA, 28 bp, Artificial Sequence
  synthetic
tggatgttggggtacattaccacaggaa

SEQ ID NO: 51 - DNA, 28 bp, Artificial Sequence
  synthetic
ggatgttggggtacattaccacaggaac

SEQ ID NO: 52 - DNA, 28 bp, Artificial Sequence
  synthetic
gatgttggggtacattaccacaggaaca

SEQ ID NO: 53 - DNA, 28 bp, Artificial Sequence
  synthetic
atgttggggtacattaccacaggaacat

SEQ ID NO: 54 - DNA, 28 bp, Artificial Sequence
  synthetic
tgttggggtacattaccacaggaacata

SEQ ID NO: 55 - DNA, 53 bp, Artificial Sequence
  synthetic
tgtggtattgggggccaagtctgtatcgcatcttgagtccctttttaccgctg

SEQ ID NO: 56 - DNA, 28 bp, Artificial Sequence
  synthetic
tgtggtattgggggccaagtctgtatcg

SEQ ID NO: 57 - DNA, 28 bp, Artificial Sequence
  synthetic
gtggtattgggggccaagtctgtatcgc

SEQ ID NO: 58 - DNA, 28 bp, Artificial Sequence
  synthetic
tggtattgggggccaagtctgtatcgca

SEQ ID NO: 59 - DNA, 28 bp, Artificial Sequence
  synthetic
ggtattgggggccaagtctgtatcgcat

SEQ ID NO: 60 - DNA, 28 bp, Artificial Sequence
  synthetic
gtattgggggccaagtctgtatcgcatc

SEQ ID NO: 61 - DNA, 28 bp, Artificial Sequence
  synthetic
tattgggggccaagtctgtatcgcatct

SEQ ID NO: 62 - DNA, 28 bp, Artificial Sequence
  synthetic
attgggggccaagtctgtatcgcatctt

SEQ ID NO: 63 - DNA, 28 bp, Artificial Sequence
  synthetic
ttgggggccaagtctgtatcgcatcttg

SEQ ID NO: 64 - DNA, 28 bp, Artificial Sequence
  synthetic
tgggggccaagtctgtatcgcatcttga

SEQ ID NO: 65 - DNA, 28 bp, Artificial Sequence
  synthetic
gggggccaagtctgtatcgcatcttgag

SEQ ID NO: 66 - DNA, 28 bp, Artificial Sequence
  synthetic
ggggccaagtctgtatcgcatcttgagt

SEQ ID NO: 67 - DNA, 28 bp, Artificial Sequence
  synthetic
gggccaagtctgtatcgcatcttgagtc

SEQ ID NO: 68 - DNA, 28 bp, Artificial Sequence
  synthetic
ggccaagtctgtatcgcatcttgagtcc

SEQ ID NO: 69 - DNA, 28 bp, Artificial Sequence
  synthetic
gccaagtctgtatcgcatcttgagtccc

SEQ ID NO: 70 - DNA, 28 bp, Artificial Sequence
  synthetic
ccaagtctgtatcgcatcttgagtccct

SEQ ID NO: 71 - DNA, 28 bp, Artificial Sequence
  synthetic
caagtctgtatcgcatcttgagtccctt

SEQ ID NO: 72 - DNA, 28 bp, Artificial Sequence
  synthetic
aagtctgtatcgcatcttgagtcccttt

SEQ ID NO: 73 - DNA, 28 bp, Artificial Sequence
  synthetic
agtctgtatcgcatcttgagtccctttt

SEQ ID NO: 74 - DNA, 28 bp, Artificial Sequence
  synthetic
gtctgtatcgcatcttgagtcccttttt

SEQ ID NO: 75 - DNA, 28 bp, Artificial Sequence
  synthetic
tctgtatcgcatcttgagtcccttttta

SEQ ID NO: 76 - DNA, 28 bp, Artificial Sequence
  synthetic
ctgtatcgcatcttgagtccctttttac

SEQ ID NO: 77 - DNA, 28 bp, Artificial Sequence
  synthetic
tgtatcgcatcttgagtccctttttacc

SEQ ID NO: 78 - DNA, 28 bp, Artificial Sequence
  synthetic
gtatcgcatcttgagtccctttttaccg

SEQ ID NO: 79 - DNA, 28 bp, Artificial Sequence
  synthetic
tatcgcatcttgagtccctttttaccgc

SEQ ID NO: 80 - DNA, 28 bp, Artificial Sequence
  synthetic
atcgcatcttgagtccctttttaccgct

SEQ ID NO: 81 - DNA, 28 bp, Artificial Sequence
  synthetic
tcgcatcttgagtccctttttaccgctg

SEQ ID NO: 82 - DNA, 53 bp, Artificial Sequence
  synthetic
gactgtttgtttaaagactgggaggatttgggggaggagattaggttaatgat

SEQ ID NO: 83 - DNA, 28 bp, Artificial Sequence
  synthetic
gactgtttgtttaaagactgggaggatt

SEQ ID NO: 84 - DNA, 28 bp, Artificial Sequence
  synthetic
actgtttgtttaaagactgggaggattt

SEQ ID NO: 85 - DNA, 28 bp, Artificial Sequence
  synthetic
ctgtttgtttaaagactgggaggatttg

SEQ ID NO: 86 - DNA, 28 bp, Artificial Sequence
  synthetic
tgtttgtttaaagactgggaggatttgg

SEQ ID NO: 87 - DNA, 28 bp, Artificial Sequence
  synthetic
gtttgtttaaagactgggaggatttggg

SEQ ID NO: 88 - DNA, 28 bp, Artificial Sequence
  synthetic
tttgtttaaagactgggaggatttgggg

SEQ ID NO: 89 - DNA, 28 bp, Artificial Sequence
  synthetic
ttgtttaaagactgggaggatttggggg

SEQ ID NO: 90 - DNA, 28 bp, Artificial Sequence
  synthetic
tgtttaaagactgggaggatttggggga

SEQ ID NO: 91 - DNA, 28 bp, Artificial Sequence
  synthetic
gtttaaagactgggaggatttgggggag

SEQ ID NO: 92 - DNA, 28 bp, Artificial Sequence
  synthetic
tttaaagactgggaggatttgggggagg

SEQ ID NO: 93 - DNA, 28 bp, Artificial Sequence
  synthetic
ttaaagactgggaggatttgggggagga

SEQ ID NO: 94 - DNA, 28 bp, Artificial Sequence
  synthetic
taaagactgggaggatttgggggaggag

SEQ ID NO: 95 - DNA, 28 bp, Artificial Sequence
  synthetic
aaagactgggaggatttgggggaggaga

SEQ ID NO: 96 - DNA, 28 bp, Artificial Sequence
  synthetic
aagactgggaggatttgggggaggagat

SEQ ID NO: 97 - DNA, 28 bp, Artificial Sequence
  synthetic
agactgggaggatttgggggaggagatt

SEQ ID NO: 98 - DNA, 28 bp, Artificial Sequence
  synthetic
gactgggaggatttgggggaggagatta

SEQ ID NO: 99 - DNA, 28 bp, Artificial Sequence
  synthetic
actgggaggatttgggggaggagattag

SEQ ID NO: 100 - DNA, 28 bp, Artificial Sequence
  synthetic
ctgggaggatttgggggaggagattagg

SEQ ID NO: 101 - DNA, 28 bp, Artificial Sequence
  synthetic
tgggaggatttgggggaggagattaggt

SEQ ID NO: 102 - DNA, 28 bp, Artificial Sequence
  synthetic
gggaggatttgggggaggagattaggtt

SEQ ID NO: 103 - DNA, 28 bp, Artificial Sequence
  synthetic
ggaggatttgggggaggagattaggtta

SEQ ID NO: 104 - DNA, 28 bp, Artificial Sequence
  synthetic
gaggatttgggggaggagattaggttaa

SEQ ID NO: 105 - DNA, 28 bp, Artificial Sequence
  synthetic
aggatttgggggaggagattaggttaat

SEQ ID NO: 106 - DNA, 28 bp, Artificial Sequence
  synthetic
ggatttgggggaggagattaggttaatg

SEQ ID NO: 107 - DNA, 28 bp, Artificial Sequence
  synthetic
gatttgggggaggagattaggttaatga

SEQ ID NO: 108 - DNA, 28 bp, Artificial Sequence
  synthetic
atttgggggaggagattaggttaatgat

SEQ ID NO: 109 - DNA, 53 bp, Artificial Sequence
  synthetic
aaagactgggaggagttgggggaggagnttaggttaatgatctttgtactagg

SEQ ID NO: 110 - DNA, 28 bp, Artificial Sequence
  synthetic
aaagactgggaggagttgggggaggagn

SEQ ID NO: 111 - DNA, 28 bp, Artificial Sequence
  synthetic
aagactgggaggagttgggggaggagnt

SEQ ID NO: 112 - DNA, 28 bp, Artificial Sequence
  synthetic
agactgggaggagttgggggaggagntt

SEQ ID NO: 113 - DNA, 28 bp, Artificial Sequence
  synthetic
gactgggaggagttgggggaggagntta

SEQ ID NO: 114 - DNA, 28 bp, Artificial Sequence
  synthetic
actgggaggagttgggggaggagnttag

SEQ ID NO: 115 - DNA, 28 bp, Artificial Sequence
  synthetic
ctgggaggagttgggggaggagnttagg

SEQ ID NO: 116 - DNA, 28 bp, Artificial Sequence
  synthetic
tgggaggagttgggggaggagnttaggt

SEQ ID NO: 117 - DNA, 28 bp, Artificial Sequence
  synthetic
gggaggagttgggggaggagnttaggtt

SEQ ID NO: 118 - DNA, 28 bp, Artificial Sequence
  synthetic
ggaggagttgggggaggagnttaggtta

SEQ ID NO: 119 - DNA, 28 bp, Artificial Sequence
  synthetic
gaggagttgggggaggagnttaggttaa

SEQ ID NO: 120 - DNA, 28 bp, Artificial Sequence
  synthetic
aggagttgggggaggagnttaggttaat

SEQ ID NO: 121 - DNA, 28 bp, Artificial Sequence
  synthetic
ggagttgggggaggagnttaggttaatg

SEQ ID NO: 122 - DNA, 28 bp, Artificial Sequence
  synthetic
gagttgggggaggagnttaggttaatga

SEQ ID NO: 123 - DNA, 28 bp, Artificial Sequence
  synthetic
agttgggggaggagnttaggttaatgat

SEQ ID NO: 124 - DNA, 28 bp, Artificial Sequence
  synthetic
gttgggggaggagnttaggttaatgatc

SEQ ID NO: 125 - DNA, 28 bp, Artificial Sequence
  synthetic
ttgggggaggagnttaggttaatgatct

SEQ ID NO: 126 - DNA, 28 bp, Artificial Sequence
  synthetic
tgggggaggagnttaggttaatgatctt

SEQ ID NO: 127 - DNA, 28 bp, Artificial Sequence
  synthetic
gggggaggagnttaggttaatgatcttt

SEQ ID NO: 128 - DNA, 28 bp, Artificial Sequence
  synthetic
ggggaggagnttaggttaatgatctttg

SEQ ID NO: 129 - DNA, 28 bp, Artificial Sequence
  synthetic
gggaggagnttaggttaatgatctttgt

SEQ ID NO: 130 - DNA, 28 bp, Artificial Sequence
  synthetic
ggaggagnttaggttaatgatctttgta

SEQ ID NO: 131 - DNA, 28 bp, Artificial Sequence
  synthetic
gaggagnttaggttaatgatctttgtac

SEQ ID NO: 132 - DNA, 28 bp, Artificial Sequence
  synthetic
aggagnttaggttaatgatctttgtact

SEQ ID NO: 133 - DNA, 28 bp, Artificial Sequence
  synthetic
ggagnttaggttaatgatctttgtacta

SEQ ID NO: 134 - DNA, 28 bp, Artificial Sequence
  synthetic
gagnttaggttaatgatctttgtactag

SEQ ID NO: 135 - DNA, 28 bp, Artificial Sequence
  synthetic
agnttaggttaatgatctttgtactagg

SEQ ID NO: 136 - DNA, 53 bp, Artificial Sequence
  synthetic
ttgggtggctttaggacatggacatcgacccttataaagaatttggagcttcc

SEQ ID NO: 137 - DNA, 28 bp, Artificial Sequence
  synthetic
ttgggtggctttaggacatggacatcga

SEQ ID NO: 138 - DNA, 28 bp, Artificial Sequence
  synthetic
tgggtggctttaggacatggacatcgac

SEQ ID NO: 139 - DNA, 28 bp, Artificial Sequence
  synthetic
gggtggctttaggacatggacatcgacc

SEQ ID NO: 140 - DNA, 28 bp, Artificial Sequence
  synthetic
ggtggctttaggacatggacatcgaccc

SEQ ID NO: 141 - DNA, 28 bp, Artificial Sequence
  synthetic
gtggctttaggacatggacatcgaccct

SEQ ID NO: 142 - DNA, 28 bp, Artificial Sequence
  synthetic
tggctttaggacatggacatcgaccctt

SEQ ID NO: 143 - DNA, 28 bp, Artificial Sequence
  synthetic
ggctttaggacatggacatcgaccctta

SEQ ID NO: 144 - DNA, 28 bp, Artificial Sequence
  synthetic
gctttaggacatggacatcgacccttat

SEQ ID NO: 145 - DNA, 28 bp, Artificial Sequence
  synthetic
ctttaggacatggacatcgacccttata

SEQ ID NO: 146 - DNA, 28 bp, Artificial Sequence
  synthetic
tttaggacatggacatcgacccttataa

SEQ ID NO: 147 - DNA, 28 bp, Artificial Sequence
  synthetic
ttaggacatggacatcgacccttataaa

SEQ ID NO: 148 - DNA, 28 bp, Artificial Sequence
  synthetic
taggacatggacatcgacccttataaag

SEQ ID NO: 149 - DNA, 28 bp, Artificial Sequence
  synthetic
aggacatggacatcgacccttataaaga

SEQ ID NO: 150 - DNA, 28 bp, Artificial Sequence
  synthetic
ggacatggacatcgacccttataaagaa

SEQ ID NO: 151 - DNA, 28 bp, Artificial Sequence
  synthetic
gacatggacatcgacccttataaagaat

SEQ ID NO: 152 - DNA, 28 bp, Artificial Sequence
  synthetic
acatggacatcgacccttataaagaatt

SEQ ID NO: 153 - DNA, 28 bp, Artificial Sequence
  synthetic
catggacatcgacccttataaagaattt

SEQ ID NO: 154 - DNA, 28 bp, Artificial Sequence
  synthetic
atggacatcgacccttataaagaatttg

SEQ ID NO: 155 - DNA, 28 bp, Artificial Sequence
  synthetic
tggacatcgacccttataaagaatttgg

SEQ ID NO: 156 - DNA, 28 bp, Artificial Sequence
  synthetic
ggacatcgacccttataaagaatttgga

SEQ ID NO: 157 - DNA, 28 bp, Artificial Sequence
  synthetic
gacatcgacccttataaagaatttggag

SEQ ID NO: 158 - DNA, 28 bp, Artificial Sequence
  synthetic
acatcgacccttataaagaatttggagc

SEQ ID NO: 159 - DNA, 28 bp, Artificial Sequence
  synthetic
catcgacccttataaagaatttggagct

SEQ ID NO: 160 - DNA, 28 bp, Artificial Sequence
  synthetic
atcgacccttataaagaatttggagctt

SEQ ID NO: 161 - DNA, 28 bp, Artificial Sequence
  synthetic
tcgacccttataaagaatttggagcttc

SEQ ID NO: 162 - DNA, 28 bp, Artificial Sequence
  synthetic
cgacccttataaagaatttggagcttcc

SEQ ID NO: 163 - DNA, 53 bp, Artificial Sequence
  synthetic
ctgtggagttactctcttttttgccgtctgacttctttccttctattcgagat

SEQ ID NO: 164 - DNA, 28 bp, Artificial Sequence
  synthetic
ctgtggagttactctcttttttgccgtc

SEQ ID NO: 165 - DNA, 28 bp, Artificial Sequence
  synthetic
tgtggagttactctcttttttgccgtct

SEQ ID NO: 166 - DNA, 28 bp, Artificial Sequence
  synthetic
gtggagttactctcttttttgccgtctg

SEQ ID NO: 167 - DNA, 28 bp, Artificial Sequence
  synthetic
tggagttactctcttttttgccgtctga

SEQ ID NO: 168 - DNA, 28 bp, Artificial Sequence
  synthetic
ggagttactctcttttttgccgtctgac

SEQ ID NO: 169 - DNA, 28 bp, Artificial Sequence
  synthetic
gagttactctcttttttgccgtctgact

SEQ ID NO: 170 - DNA, 28 bp, Artificial Sequence
  synthetic
agttactctcttttttgccgtctgactt

SEQ ID NO: 171 - DNA, 28 bp, Artificial Sequence
  synthetic
gttactctcttttttgccgtctgacttc

SEQ ID NO: 172 - DNA, 28 bp, Artificial Sequence
  synthetic
ttactctcttttttgccgtctgacttct

SEQ ID NO: 173 - DNA, 28 bp, Artificial Sequence
  synthetic
tactctcttttttgccgtctgacttctt

SEQ ID NO: 174 - DNA, 28 bp, Artificial Sequence
  synthetic
actctcttttttgccgtctgacttcttt

SEQ ID NO: 175 - DNA, 28 bp, Artificial Sequence
  synthetic
ctctcttttttgccgtctgacttctttc

SEQ ID NO: 176 - DNA, 28 bp, Artificial Sequence
  synthetic
tctcttttttgccgtctgacttctttcc

SEQ ID NO: 177 - DNA, 28 bp, Artificial Sequence
  synthetic
ctcttttttgccgtctgacttctttcct

SEQ ID NO: 178 - DNA, 28 bp, Artificial Sequence
  synthetic
tcttttttgccgtctgacttctttcctt

SEQ ID NO: 179 - DNA, 28 bp, Artificial Sequence
  synthetic
cttttttgccgtctgacttctttccttc

SEQ ID NO: 180 - DNA, 28 bp, Artificial Sequence
  synthetic
ttttttgccgtctgacttctttccttct

SEQ ID NO: 181 - DNA, 28 bp, Artificial Sequence
  synthetic
tttttgccgtctgacttctttccttcta

SEQ ID NO: 182 - DNA, 28 bp, Artificial Sequence
  synthetic
ttttgccgtctgacttctttccttctat

SEQ ID NO: 183 - DNA, 28 bp, Artificial Sequence
  synthetic
tttgccgtctgacttctttccttctatt

SEQ ID NO: 184 - DNA, 28 bp, Artificial Sequence
  synthetic
ttgccgtctgacttctttccttctattc

SEQ ID NO: 185 - DNA, 28 bp, Artificial Sequence
  synthetic
tgccgtctgacttctttccttctattcg

SEQ ID NO: 186 - DNA, 28 bp, Artificial Sequence
  synthetic
gccgtctgacttctttccttctattcga

SEQ ID NO: 187 - DNA, 28 bp, Artificial Sequence
  synthetic
ccgtctgacttctttccttctattcgag

SEQ ID NO: 188 - DNA, 28 bp, Artificial Sequence
  synthetic
cgtctgacttctttccttctattcgaga

SEQ ID NO: 189 - DNA, 28 bp, Artificial Sequence
  synthetic
gtctgacttctttccttctattcgagat

SEQ ID NO: 190 - DNA, 53 bp, Artificial Sequence
  synthetic
ctgtggagttactctcttttttgcctgctgacttctttccttctattcgagat

SEQ ID NO: 191 - DNA, 28 bp, Artificial Sequence
  synthetic
ctgtggagttactctcttttttgcctgc

SEQ ID NO: 192 - DNA, 28 bp, Artificial Sequence
  synthetic
tgtggagttactctcttttttgcctgct

SEQ ID NO: 193 - DNA, 28 bp, Artificial Sequence
  synthetic
gtggagttactctcttttttgcctgctg

SEQ ID NO: 194 - DNA, 28 bp, Artificial Sequence
  synthetic
tggagttactctcttttttgcctgctga

SEQ ID NO: 195 - DNA, 28 bp, Artificial Sequence
  synthetic
ggagttactctcttttttgcctgctgac

SEQ ID NO: 196 - DNA, 28 bp, Artificial Sequence
  synthetic
gagttactctcttttttgcctgctgact

SEQ ID NO: 197 - DNA, 28 bp, Artificial Sequence
  synthetic
agttactctcttttttgcctgctgactt

SEQ ID NO: 198 - DNA, 28 bp, Artificial Sequence
  synthetic
gttactctcttttttgcctgctgacttc

SEQ ID NO: 199 - DNA, 28 bp, Artificial Sequence
  synthetic
ttactctcttttttgcctgctgacttct

SEQ ID NO: 200 - DNA, 28 bp, Artificial Sequence
  synthetic
tactctcttttttgcctgctgacttctt

SEQ ID NO: 201 - DNA, 28 bp, Artificial Sequence
  synthetic
actctcttttttgcctgctgacttcttt

SEQ ID NO: 202 - DNA, 28 bp, Artificial Sequence
  synthetic
ctctcttttttgcctgctgacttctttc

SEQ ID NO: 203 - DNA, 28 bp, Artificial Sequence
  synthetic
tctcttttttgcctgctgacttctttcc

SEQ ID NO: 204 - DNA, 28 bp, Artificial Sequence
  synthetic
ctcttttttgcctgctgacttctttcct

SEQ ID NO: 205 - DNA, 28 bp, Artificial Sequence
  synthetic
tcttttttgcctgctgacttctttcctt

SEQ ID NO: 206 - DNA, 28 bp, Artificial Sequence
  synthetic
cttttttgcctgctgacttctttccttc

SEQ ID NO: 207 - DNA, 28 bp, Artificial Sequence
  synthetic
ttttttgcctgctgacttctttccttct

SEQ ID NO: 208 - DNA, 28 bp, Artificial Sequence
  synthetic
tttttgcctgctgacttctttccttcta

SEQ ID NO: 209 - DNA, 28 bp, Artificial Sequence
  synthetic
ttttgcctgctgacttctttccttctat

SEQ ID NO: 210 - DNA, 28 bp, Artificial Sequence
  synthetic
tttgcctgctgacttctttccttctatt

SEQ ID NO: 211 - DNA, 28 bp, Artificial Sequence
  synthetic
ttgcctgctgacttctttccttctattc

SEQ ID NO: 212 - DNA, 28 bp, Artificial Sequence
  synthetic
tgcctgctgacttctttccttctattcg

SEQ ID NO: 213 - DNA, 28 bp, Artificial Sequence
  synthetic
gcctgctgacttctttccttctattcga

SEQ ID NO: 214 - DNA, 28 bp, Artificial Sequence
  synthetic
cctgctgacttctttccttctattcgag

SEQ ID NO: 215 - DNA, 28 bp, Artificial Sequence
  synthetic
ctgctgacttctttccttctattcgaga

SEQ ID NO: 216 - DNA, 28 bp, Artificial Sequence
  synthetic
tgctgacttctttccttctattcgagat

SEQ ID NO: 217 - DNA, 53 bp, Artificial Sequence
  synthetic
ctgtggagttactctcttttttgcctactgacttctttccttctattcgagat

SEQ ID NO: 218 - DNA, 28 bp, Artificial Sequence
  synthetic
ctgtggagttactctcttttttgcctac

SEQ ID NO: 219 - DNA, 28 bp, Artificial Sequence
  synthetic
tgtggagttactctcttttttgcctact

SEQ ID NO: 220 - DNA, 28 bp, Artificial Sequence
  synthetic
gtggagttactctcttttttgcctactg

SEQ ID NO: 221 - DNA, 28 bp, Artificial Sequence
  synthetic
tggagttactctcttttttgcctactga

SEQ ID NO: 222 - DNA, 28 bp, Artificial Sequence
  synthetic
ggagttactctcttttttgcctactgac

SEQ ID NO: 223 - DNA, 28 bp, Artificial Sequence
  synthetic
gagttactctcttttttgcctactgact

SEQ ID NO: 224 - DNA, 28 bp, Artificial Sequence
  synthetic
agttactctcttttttgcctactgactt

SEQ ID NO: 225 - DNA, 28 bp, Artificial Sequence
  synthetic
gttactctcttttttgcctactgacttc

SEQ ID NO: 226 - DNA, 28 bp, Artificial Sequence
  synthetic
ttactctcttttttgcctactgacttct

SEQ ID NO: 227 - DNA, 28 bp, Artificial Sequence
  synthetic
tactctcttttttgcctactgacttctt

SEQ ID NO: 228 - DNA, 28 bp, Artificial Sequence
  synthetic
actctcttttttgcctactgacttcttt

SEQ ID NO: 229 - DNA, 28 bp, Artificial Sequence
  synthetic
ctctcttttttgcctactgacttctttc

SEQ ID NO: 230 - DNA, 28 bp, Artificial Sequence
  synthetic
tctcttttttgcctactgacttctttcc

SEQ ID NO: 231 - DNA, 28 bp, Artificial Sequence
  synthetic
ctcttttttgcctactgacttctttcct

SEQ ID NO: 232 - DNA, 28 bp, Artificial Sequence
  synthetic
tcttttttgcctactgacttctttcctt

SEQ ID NO: 233 - DNA, 28 bp, Artificial Sequence
  synthetic
cttttttgcctactgacttctttccttc

SEQ ID NO: 234 - DNA, 28 bp, Artificial Sequence
  synthetic
ttttttgcctactgacttctttccttct

SEQ ID NO: 235 - DNA, 28 bp, Artificial Sequence
  synthetic
tttttgcctactgacttctttccttcta

SEQ ID NO: 236 - DNA, 28 bp, Artificial Sequence
  synthetic
ttttgcctactgacttctttccttctat

SEQ ID NO: 237 - DNA, 28 bp, Artificial Sequence
  synthetic
tttgcctactgacttctttccttctatt

SEQ ID NO: 238 - DNA, 28 bp, Artificial Sequence
  synthetic
ttgcctactgacttctttccttctattc

SEQ ID NO: 239 - DNA, 28 bp, Artificial Sequence
  synthetic
tgcctactgacttctttccttctattcg

SEQ ID NO: 240 - DNA, 28 bp, Artificial Sequence
  synthetic
gcctactgacttctttccttctattcga

SEQ ID NO: 241 - DNA, 28 bp, Artificial Sequence
  synthetic
cctactgacttctttccttctattcgag

SEQ ID NO: 242 - DNA, 28 bp, Artificial Sequence
  synthetic
ctactgacttctttccttctattcgaga

SEQ ID NO: 243 - DNA, 28 bp, Artificial Sequence
  synthetic
tactgacttctttccttctattcgagat

SEQ ID NO: 244 - DNA, 53 bp, Artificial Sequence
  synthetic
gactgtttgtttaaagactgggaggctttgggggaggagattaggttaatgat

SEQ ID NO: 245 - DNA, 28 bp, Artificial Sequence
  synthetic
gactgtttgtttaaagactgggaggctt

SEQ ID NO: 246 - DNA, 28 bp, Artificial Sequence
  synthetic
actgtttgtttaaagactgggaggcttt

SEQ ID NO: 247 - DNA, 28 bp, Artificial Sequence
  synthetic
ctgtttgtttaaagactgggaggctttg

SEQ ID NO: 248 - DNA, 28 bp, Artificial Sequence
  synthetic
tgtttgtttaaagactgggaggctttgg

SEQ ID NO: 249 - DNA, 28 bp, Artificial Sequence
  synthetic
gtttgtttaaagactgggaggctttggg

SEQ ID NO: 250 - DNA, 28 bp, Artificial Sequence
  synthetic
tttgtttaaagactgggaggctttgggg

SEQ ID NO: 251 - DNA, 28 bp, Artificial Sequence
  synthetic
ttgtttaaagactgggaggctttggggg

SEQ ID NO: 252 - DNA, 28 bp, Artificial Sequence
  synthetic
tgtttaaagactgggaggctttggggga

SEQ ID NO: 253 - DNA, 28 bp, Artificial Sequence
  synthetic
gtttaaagactgggaggctttgggggag

SEQ ID NO: 254 - DNA, 28 bp, Artificial Sequence
  synthetic
tttaaagactgggaggctttgggggagg

SEQ ID NO: 255 - DNA, 28 bp, Artificial Sequence
  synthetic
ttaaagactgggaggctttgggggagga

SEQ ID NO: 256 - DNA, 28 bp, Artificial Sequence
  synthetic
taaagactgggaggctttgggggaggag

SEQ ID NO: 257 - DNA, 28 bp, Artificial Sequence
  synthetic
aaagactgggaggctttgggggaggaga

SEQ ID NO: 258 - DNA, 28 bp, Artificial Sequence
  synthetic
aagactgggaggctttgggggaggagat

SEQ ID NO: 259 - DNA, 28 bp, Artificial Sequence
  synthetic
agactgggaggctttgggggaggagatt

SEQ ID NO: 260 - DNA, 28 bp, Artificial Sequence
  synthetic
gactgggaggctttgggggaggagatta

SEQ ID NO: 261 - DNA, 28 bp, Artificial Sequence
  synthetic
actgggaggctttgggggaggagattag

SEQ ID NO: 262 - DNA, 28 bp, Artificial Sequence
  synthetic
ctgggaggctttgggggaggagattagg

SEQ ID NO: 263 - DNA, 28 bp, Artificial Sequence
  synthetic
tgggaggctttgggggaggagattaggt

SEQ ID NO: 264 - DNA, 28 bp, Artificial Sequence
  synthetic
gggaggctttgggggaggagattaggtt

SEQ ID NO: 265 - DNA, 28 bp, Artificial Sequence
  synthetic
ggaggctttgggggaggagattaggtta

SEQ ID NO: 266 - DNA, 28 bp, Artificial Sequence
  synthetic
gaggctttgggggaggagattaggttaa

SEQ ID NO: 267 - DNA, 28 bp, Artificial Sequence
  synthetic
aggctttgggggaggagattaggttaat

SEQ ID NO: 268 - DNA, 28 bp, Artificial Sequence
  synthetic
ggctttgggggaggagattaggttaatg

SEQ ID NO: 269 - DNA, 28 bp, Artificial Sequence
  synthetic
gctttgggggaggagattaggttaatga

SEQ ID NO: 270 - DNA, 28 bp, Artificial Sequence
  synthetic
ctttgggggaggagattaggttaatgat

SEQ ID NO: 271 - DNA, 53 bp, Artificial Sequence
  synthetic
aaagactgggaggagttgggggagggggttaggttaatgatctttgtactagg

SEQ ID NO: 272 - DNA, 28 bp, Artificial Sequence
  synthetic
aaagactgggaggagttgggggaggggg

SEQ ID NO: 273 - DNA, 28 bp, Artificial Sequence
  synthetic
aagactgggaggagttgggggagggggt

SEQ ID NO: 274 - DNA, 28 bp, Artificial Sequence
  synthetic
agactgggaggagttgggggagggggtt

SEQ ID NO: 275 - DNA, 28 bp, Artificial Sequence
  synthetic
gactgggaggagttgggggagggggtta

SEQ ID NO: 276 - DNA, 28 bp, Artificial Sequence
  synthetic
actgggaggagttgggggagggggttag

SEQ ID NO: 277 - DNA, 28 bp, Artificial Sequence
  synthetic
ctgggaggagttgggggagggggttagg

SEQ ID NO: 278 - DNA, 28 bp, Artificial Sequence
  synthetic
tgggaggagttgggggagggggttaggt

SEQ ID NO: 279 - DNA, 28 bp, Artificial Sequence
  synthetic
gggaggagttgggggagggggttaggtt

SEQ ID NO: 280 - DNA, 28 bp, Artificial Sequence
  synthetic
ggaggagttgggggagggggttaggtta

SEQ ID NO: 281 - DNA, 28 bp, Artificial Sequence
  synthetic
gaggagttgggggagggggttaggttaa

SEQ ID NO: 282 - DNA, 28 bp, Artificial Sequence
  synthetic
aggagttgggggagggggttaggttaat

SEQ ID NO: 283 - DNA, 28 bp, Artificial Sequence
  synthetic
ggagttgggggagggggttaggttaatg

SEQ ID NO: 284 - DNA, 28 bp, Artificial Sequence
  synthetic
gagttgggggagggggttaggttaatga

SEQ ID NO: 285 - DNA, 28 bp, Artificial Sequence
  synthetic
agttgggggagggggttaggttaatgat

SEQ ID NO: 286 - DNA, 28 bp, Artificial Sequence
  synthetic
gttgggggagggggttaggttaatgatc

SEQ ID NO: 287 - DNA, 28 bp, Artificial Sequence
  synthetic
ttgggggagggggttaggttaatgatct

SEQ ID NO: 288 - DNA, 28 bp, Artificial Sequence
  synthetic
tgggggagggggttaggttaatgatctt

SEQ ID NO: 289 - DNA, 28 bp, Artificial Sequence
  synthetic
gggggagggggttaggttaatgatcttt

SEQ ID NO: 290 - DNA, 28 bp, Artificial Sequence
  synthetic
ggggagggggttaggttaatgatctttg

SEQ ID NO: 291 - DNA, 28 bp, Artificial Sequence
  synthetic
gggagggggttaggttaatgatctttgt

SEQ ID NO: 292 - DNA, 28 bp, Artificial Sequence
  synthetic
ggagggggttaggttaatgatctttgta

SEQ ID NO: 293 - DNA, 28 bp, Artificial Sequence
  synthetic
gagggggttaggttaatgatctttgtac

SEQ ID NO: 294 - DNA, 28 bp, Artificial Sequence
  synthetic
agggggttaggttaatgatctttgtact

SEQ ID NO: 295 - DNA, 28 bp, Artificial Sequence
  synthetic
gggggttaggttaatgatctttgtacta

SEQ ID NO: 296 - DNA, 28 bp, Artificial Sequence
  synthetic
ggggttaggttaatgatctttgtactag

SEQ ID NO: 297 - DNA, 28 bp, Artificial Sequence
  synthetic
gggttaggttaatgatctttgtactagg

SEQ ID NO: 298 - DNA, 53 bp, Artificial Sequence
  synthetic
ttgggtggctttaggacatggacatggtcccttataaagaatttggagcttcc

SEQ ID NO: 299 - DNA, 28 bp, Artificial Sequence
  synthetic
ttgggtggctttaggacatggacatggt

SEQ ID NO: 300 - DNA, 28 bp, Artificial Sequence
  synthetic
tgggtggctttaggacatggacatggtc

SEQ ID NO: 301 - DNA, 28 bp, Artificial Sequence
  synthetic
gggtggctttaggacatggacatggtcc

SEQ ID NO: 302 - DNA, 28 bp, Artificial Sequence
  synthetic
ggtggctttaggacatggacatggtccc

SEQ ID NO: 303 - DNA, 28 bp, Artificial Sequence
  synthetic
gtggctttaggacatggacatggtccct

SEQ ID NO: 304 - DNA, 28 bp, Artificial Sequence
  synthetic
tggctttaggacatggacatggtccctt

SEQ ID NO: 305 - DNA, 28 bp, Artificial Sequence
  synthetic
ggctttaggacatggacatggtccctta

SEQ ID NO: 306 - DNA, 28 bp, Artificial Sequence
  synthetic
gctttaggacatggacatggtcccttat

SEQ ID NO: 307 - DNA, 28 bp, Artificial Sequence
  synthetic
ctttaggacatggacatggtcccttata

SEQ ID NO: 308 - DNA, 28 bp, Artificial Sequence
  synthetic
tttaggacatggacatggtcccttataa

SEQ ID NO: 309 - DNA, 28 bp, Artificial Sequence
  synthetic
ttaggacatggacatggtcccttataaa

SEQ ID NO: 310 - DNA, 28 bp, Artificial Sequence
  synthetic
taggacatggacatggtcccttataaag

SEQ ID NO: 311 - DNA, 28 bp, Artificial Sequence
  synthetic
aggacatggacatggtcccttataaaga

SEQ ID NO: 312 - DNA, 28 bp, Artificial Sequence
  synthetic
ggacatggacatggtcccttataaagaa

SEQ ID NO: 313 - DNA, 28 bp, Artificial Sequence
  synthetic
gacatggacatggtcccttataaagaat

SEQ ID NO: 314 - DNA, 28 bp, Artificial Sequence
  synthetic
acatggacatggtcccttataaagaatt

SEQ ID NO: 315 - DNA, 28 bp, Artificial Sequence
  synthetic
catggacatggtcccttataaagaattt

SEQ ID NO: 316 - DNA, 28 bp, Artificial Sequence
  synthetic
atggacatggtcccttataaagaatttg

SEQ ID NO: 317 - DNA, 28 bp, Artificial Sequence
  synthetic
tggacatggtcccttataaagaatttgg

SEQ ID NO: 318 - DNA, 28 bp, Artificial Sequence
  synthetic
ggacatggtcccttataaagaatttgga

SEQ ID NO: 319 - DNA, 28 bp, Artificial Sequence
  synthetic
gacatggtcccttataaagaatttggag

SEQ ID NO: 320 - DNA, 28 bp, Artificial Sequence
  synthetic
acatggtcccttataaagaatttggagc

SEQ ID NO: 321 - DNA, 28 bp, Artificial Sequence
  synthetic
catggtcccttataaagaatttggagct

SEQ ID NO: 322 - DNA, 28 bp, Artificial Sequence
  synthetic
atggtcccttataaagaatttggagctt

SEQ ID NO: 323 - DNA, 28 bp, Artificial Sequence
  synthetic
tggtcccttataaagaatttggagcttc

SEQ ID NO: 324 - DNA, 28 bp, Artificial Sequence
  synthetic
ggtcccttataaagaatttggagcttcc

SEQ ID NO: 325 - DNA, 53 bp, Artificial Sequence
  synthetic
ctgtggagttactctcttttttgcctaatgacttctttccttctattcgagat

SEQ ID NO: 326 - DNA, 28 bp, Artificial Sequence
  synthetic
ctgtggagttactctcttttttgcctaa

SEQ ID NO: 327 - DNA, 28 bp, Artificial Sequence
  synthetic
tgtggagttactctcttttttgcctaat

SEQ ID NO: 328 - DNA, 28 bp, Artificial Sequence
  synthetic
gtggagttactctcttttttgcctaatg

SEQ ID NO: 329 - DNA, 28 bp, Artificial Sequence
  synthetic
tggagttactctcttttttgcctaatga

SEQ ID NO: 330 - DNA, 28 bp, Artificial Sequence
  synthetic
ggagttactctcttttttgcctaatgac

SEQ ID NO: 331 - DNA, 28 bp, Artificial Sequence
  synthetic
gagttactctcttttttgcctaatgact

SEQ ID NO: 332 - DNA, 28 bp, Artificial Sequence
  synthetic
agttactctcttttttgcctaatgactt

SEQ ID NO: 333 - DNA, 28 bp, Artificial Sequence
  synthetic
gttactctcttttttgcctaatgacttc

SEQ ID NO: 334 - DNA, 28 bp, Artificial Sequence
  synthetic
ttactctcttttttgcctaatgacttct

SEQ ID NO: 335 - DNA, 28 bp, Artificial Sequence
  synthetic
tactctcttttttgcctaatgacttctt

SEQ ID NO: 336 - DNA, 28 bp, Artificial Sequence
  synthetic
actctcttttttgcctaatgacttcttt

SEQ ID NO: 337 - DNA, 28 bp, Artificial Sequence
  synthetic
ctctcttttttgcctaatgacttctttc

SEQ ID NO: 338 - DNA, 28 bp, Artificial Sequence
  synthetic
tctcttttttgcctaatgacttctttcc

SEQ ID NO: 339 - DNA, 28 bp, Artificial Sequence
  synthetic
ctcttttttgcctaatgacttctttcct

SEQ ID NO: 340 - DNA, 28 bp, Artificial Sequence
  synthetic
tcttttttgcctaatgacttctttcctt

SEQ ID NO: 341 - DNA, 28 bp, Artificial Sequence
  synthetic
cttttttgcctaatgacttctttccttc

SEQ ID NO: 342 - DNA, 28 bp, Artificial Sequence
  synthetic
ttttttgcctaatgacttctttccttct

SEQ ID NO: 343 - DNA, 28 bp, Artificial Sequence
  synthetic
tttttgcctaatgacttctttccttcta

SEQ ID NO: 344 - DNA, 28 bp, Artificial Sequence
  synthetic
ttttgcctaatgacttctttccttctat

SEQ ID NO: 345 - DNA, 28 bp, Artificial Sequence
  synthetic
tttgcctaatgacttctttccttctatt

SEQ ID NO: 346 - DNA, 28 bp, Artificial Sequence
  synthetic
ttgcctaatgacttctttccttctattc

SEQ ID NO: 347 - DNA, 28 bp, Artificial Sequence
  synthetic
tgcctaatgacttctttccttctattcg

SEQ ID NO: 348 - DNA, 28 bp, Artificial Sequence
  synthetic
gcctaatgacttctttccttctattcga

SEQ ID NO: 349 - DNA, 28 bp, Artificial Sequence
  synthetic
cctaatgacttctttccttctattcgag

SEQ ID NO: 350 - DNA, 28 bp, Artificial Sequence
  synthetic
ctaatgacttctttccttctattcgaga

SEQ ID NO: 351 - DNA, 28 bp, Artificial Sequence
  synthetic
taatgacttctttccttctattcgagat

(19) United States
(12) Patent Application Publication
(10) Pub. No.: US 2005/0053916 A1
(43) Pub. Date: Mar. 10, 2005

(54) Diagnostic for long term response of HBV carrier to 3TC therapy

(76) Inventors: Brent E. Korba, Laurel, MD (US); Alessia Ciancio, Torino (IT); John L. Gerin, Bethesda, MD (US)

Correspondence Address: KING & SPALDING LLP, 191 PEACHTREE STREET, N.E., ATLANTA, GA, 30303-1763, US

(21) Appl. No.: 10/677,920

(22) Filed: Oct. 1, 2003

Related U.S. Application Data
(60) Provisional application No. 60/415,301, filed on Oct. 1, 2002.

(51) Int. Cl. C12Q 1/70; C12Q 1/68

(52) U.S. Cl. 435/5; 435/6

(57) ABSTRACT

The invention is a diagnostic, kit, and materials for the prediction of the long-term response of a chronic hepatitis B virus (HBV) carrier to therapy with 3TC (also known as lamivudine).

This application claims the benefit of priority to U.S. Provisional Application Ser. No. 60/415,301 filed Oct. 1, 2002, the disclosure of which is herein incorporated by reference.

### FIELD OF THE INVENTION

The invention is a diagnostic, kit, and materials for the prediction of the long-term response of a chronic hepatitis B virus (HBV) carrier to therapy with 3TC (also known as lamivudine).

### BACKGROUND

Hepatitis B virus (HBV) is a DNA virus in the Hepadnaviridae family which causes hepatitis in humans. Hepatitis B viral infection has reached epidemic levels around the world. Acute HBV infection can lead to hepatitis and liver damage. Most patients infected as adults typically recover from this acute infection. However, in some patients, especially those infected as infants or children, high levels of viral antigens can persist in the blood for an extended, indefinite period, causing chronic hepatitis that can persist for 20 years or more. The consequences of chronic infection are a high risk of developing severe liver disease often progressing to cirrhosis and liver cancer. Patients infected with chronic hepatitis are more common in developing countries.

The HBV genome is a partially double stranded DNA molecule approximately 3200 bases in length which is packed inside a nucleocapsid structure composed of the viral core (C) protein. This nucleocapsid is surrounded by the viral envelope, a mixture of lipids and the viral structural proteins or surface antigens. The genome is comprised of four genes: pol, env, pre-core/core and X that respectively encode the viral DNA-polymerase, envelope protein, pre-core/core protein and protein X. Antigens of HBV proteins can be detected and are often used for diagnostic purposes. The envelope protein antigen is referred to as HBsAg (hepatitis B surface antigen) and it comprises the outer surface coat of the virus. The core protein antigen is referred to as HBcAg (hepatitis B core antigen) and it forms the core of the virus that encapsulates the HBV DNA. The precore protein antigen is referred to as HBeAg (hepatitis B e antigen) which is an alternatively processed protein of the pre-core gene that is only synthesized under conditions of high viral replication; the exact function of this protein is unknown. The protein X antigen is referred to as HBxAg (hepatitis B x antigen). The exact function(s) of this protein is also unknown, although its primary role is to dramatically amplify the levels of HBV replication.

Chronic infection with HBV can be categorized as either “replicative” or “non-replicative”. In non-replicative infection, the rate of viral replication in the liver is low and serum HBV DNA concentration is generally low and HBeAg is not detected. In “replicative” infection, the patient usually has a relatively high serum concentration of viral DNA and detectable HBeAg. Patients with chronic hepatitis B and “replicative” infection defined by the presence of detectable HBeAg have a poorer prognosis generally and a greater chance of developing cirrhosis and/or hepatocellular carcinoma than those without HBeAg. In certain strains of HBV with mutations in the pre-core gene, “replicative” infection can occur in the absence of detectable serum HBeAg (HBeAg negative), and these individuals can have especially severe disease progression. Therefore, HBsAg positive patients with chronic hepatitis can be either positive or negative for serum HBeAg. HBsAg positive/HBeAg positive patients represent the classical form of chronic hepatitis B while the HBsAg positive/HBeAg negative group is more heterogeneous and includes different etiologies.

It has been estimated that hepatitis B virus has a mutation rate of approximately <2×10−1 base substitutions/site/year. The time for emergence of antiviral resistant HBV is much longer than noted for HIV, which can be due to the overlapping reading frames in the HBV genome.

Alpha-interferons were the first drugs approved in the United States for the treatment of chronic hepatitis B. Interferon treatment is recommended for individuals who have “replicative disease” (HBeAg positive). Most HBV carriers are not candidates for alpha interferon therapy, but in those who are, about 40% of such individuals will lose serum HBeAg after 16 weeks or more of treatment with interferon-alpha. Loss of HBeAg is correlated with an improved prognosis

Other treatment options for chronic hepatitis B include nucleoside analogues. In December, 1998, the United States Food and Drug Administration approved lamivudine (also known as 3TC) for the treatment of chronic hepatitis B (See U.S. Pat. No. 5,532,246). Lamivudine has been shown to be an effective and safe therapeutic agent, but because of the requirement for a long course of therapy viral resistance has become a significant issue.

On Sep. 20, 2002, the U.S. Food and Drug Administration approved adefovir dipivoxil for the treatment of chronic hepatitis B. HEPSERA™ is the trade name for adefovir dipivoxil, a diester prodrug of adefovir. Adefovir is an acyclic nucleotide analogue of adenosine monophosphate that inhibits the hepatitis B virus (HBV) DNA polymerase by competing with the natural substrate deoxyadenosine triphosphate and by causing DNA chain termination after its incorporation into viral DNA. The chemical name of adefovir dipivoxil is 9-[2-[bis[(pivaloyloxy)methoxy]phosphinyl]methoxy]-ethyl]adenine. Adefovir is phosphorylated to the active metabolite, adefovir diphosphate, by cellular kinases. See, for example, U.S. Pat. Nos. 5,641,763 and 5,142,051, entitled, N-phosphonylmethoxyalkyl derivatives of pyrimidine and purine bases and a therapeutical composition therefrom with antiviral activity.

Idenix Pharmaceuticals, Ltd. discloses 2′-deoxy-β-L-erythropentofurano-nucleosides, and their use in the treatment of HBV in U.S. Pat. Nos. 6,395,716; 6,444,652; 6,566,344 and 6,539,837. See also WO 00/09531. A method for the treatment of hepatitis B infection in humans and other host animals is disclosed that includes administering an effective amount of a biologically active 2′-deoxy-β-L-erythro-pentofuranonucleoside (alternatively referred to as β-L-dN or a β-L-2′-dN) or a pharmaceutically acceptable salt, ester or prodrug thereof, including β-L-deoxyribothymidine (β-L-dT), β-L-deoxyribocytidine (β-L-dC), β-L-deoxyribouridine (β-L-dU), β-L-deoxyribo-guanosine (β-L-dG), β-L-deoxyriboadenosine (β-L-dA) and β-L-deoxyriboinosine (β-L-dI), administered either alone or in combination, optionally in a pharmaceutically acceptable carrier.

BeAg Negative Population:

Hepatitis B e antigen (HBeAg)-negative chronic hepatitis B results from infection with hepatitis B virus mutants unable to produce HBeAg. It accounts for 7-30% of patients with chronic hepatitis B virus (HBV) worldwide, with the highest rates reported for Mediterranean Europe and Asia. Most patients appear to be infected with precore mutants of HBV that contain premature stop codons in the precore gene which prevents HBeAg production. The most frequent HBV precore variant has a point mutation at position 1896 (G to A mutation) but other mutants, deletions or genomic rearrangements may also be present.

The HBeAg-negative population represents a difficult to treat clinical group with significant disease in which licensed therapies have higher than normal failure rates (Rizzetto, J., Med. Virol. 66:435 (2002); Hadziyannis and Vassilopoulos, Hepatol. 34:617 (2001); Zoulim and Trepo, Hepatol. 32:1172, 2000). Rizzetto et al. report that patient responses to interferon treatment are lower in HBeAg-negative than in HBeAg-positive patients, whereas initial (but not long term) response rates to lamivudine are equivalent in HBeAg-negative and HBeAg-positive patients.

In some patient populations, therapy with lamivudine fails to provide long term responses to HBV. Such failures are characterized by either (i) a rebound in virus and liver disease following initial response to therapy (“breakthrough”) usually marked by the appearance of drug-resistant HBV mutants, or (ii) the lack of significant reductions in viremia and/or normalization of liver disease markers, sometimes accompanied by the appearance of drug-resistant HBV mutants (“non-responder”). Recent studies have indicated that lamivudine therapy of individuals chronically-infected with HBV precore/core promoter variants (A1762T, G1764A, G1896A) is associated with a significant reversion frequency (up to 33%) of these mutations to wild-type and, in some cases, the re-acquisition of circulating HBeAg (Hepatol., vol. 32 (2000) Lok, et al., 1145; Cho et al., 1163). Furthermore, these reversions were not correlated with the development of the primary lamivudine-resistance polymerase mutations L180M, and M204V/I (Bartholomueusz and Locamini, IVN 5:123 (1997); Pillay et al., IVN 6:9 (1998); Das et al. J. Virol. 75:4771, 2001).

Cho et al. Hepatol., vol. 32: 1163 (2000) describe that in the chronic HBV HBeAg negative patient population studied, lamivudine administration caused a reversion of the precore/core mutation to wild type virus.

Lok et al. Hepatol., vol. 32: 1145 (2000) describe that in the chronic HBV HBeAg negative patient population studied, administration of lamivudine caused a reversion of the precore mutation and reappearance of HBeAg in some of the patients.

Hadziyannis and Vassilopoulos, Hepatol. 34:617 (2001) review the efficacy of lamivudine and interferon therapy in HBeAg negative chronic hepatitis B. The authors describe that in one population almost half of the patients had virological and biochemical breakthroughs after lamivudine therapy and recommend early recognition of virological breakthroughs during lamivudine therapy.

The long-term response of HBV patients to lamivudine can vary, and in some instances may result in a more aggressive form of the disease. Viral markers have not yet been identified that can be used to predict the long-term response of a chronic hepatitis B virus (HBV) carrier to therapy with lamivudine.

There currently exists technology licensed for clinical use for the detection of changes in HIV, HBV, and HCV sequences related to drug-resistance and genotype. Assays such as Polymerase Chain Reactions (PCR) and DNA sequencing, real time PCR, DNA array technologies, including macroarray, microarray, and gene chip technology, RNA array technologies, including macroarray, microarray, and gene chip technology, protein array assays, and line probe (LiPA) assays are useful for the detection of HBV drug-resistant variants in patient serum.

PCT Publication No. WO 97/40193 to Innogenetics describes a method for typing and detecting HBV in a biological sample to allow for the detection of the different HBV mutants and genotypes in a reverse hybridization assay. This is the base technology for LiPA assays.

It is an object of the invention to identify viral markers and probes to predict the long-term response of a chronic HBV, and in particular, an HBeAg negative, carrier to therapy with lamivudine.

It is another object of the invention to provide a method to predict the long-term response of a chronic HBV carrier to therapy with lamivudine and in particular, an HBeAg negative carrier.

It is a further object of the invention to provide kits and materials useful for the detection of viral markers that predict long-term response of a chronic HBV carrier to therapy with lamivudine.

### SUMMARY OF THE INVENTION

It has been surprisingly discovered that the long-term response to therapy with lamivudine of a chronic hepatitis B virus (HBV) carrier, and in particular an HBeAg negative hepatitis B virus carrier, can be predicted.

In the discussion below, the amino acid positions for the HBV polymerase gene are numbered to be consistent with the newly established scheme designed to standardize the nomenclature for lamivudine-resistance mutations, L180M and M204V/I (originally designated as L528M or L526M, and M552V/I or M550V/I) (Stuyver, et al., Hepatology 33:751-757 (2001)). To avoid confusion, the original amino acid numbering scheme (Allen, et al., Hepatology 27:1670-1677 (1998); Pillay, et al., International Antiviral News 6(9):167-169 (1998)) is also noted as several previously described mutations discussed used that nomenclature. The numbering used here is most specific for the consensus sequence for genotype D. There are analogous sequences in other genotypes, or in variation of genotype D that may differ by a few nucleotides, however, the correlation among genotypes and variations will be obvious to one of ordinary skill in the art using conventional alignment approaches.

The presence of a leucine at amino acid position (aa) 91 (originally codon 438) and/or a cysteine at aa256 (originally codon 604) in the DNA polymerase region of HBV is highly correlated with the failure of long-term response to lamivudine. The presence of these two amino acids is not considered a mutation of the HBV genome, as the HBV consensus sequences list aa91 as both leucine and isoleucine, and aa256 as cysteine and serine. Rather these amino acids represent a natural heterogeneity in the HBV consensus sequences at the amino acid positions described. It was unexpectedly found that among a patient pool tested, all patients who carried hepatitis B virus with a leucine at aa91 instead of an isoleucine at that position demonstrated a failure in long term lamivudine therapy, i.e., were diagnosed over time with a rebound in virus titer or no substantive change in virus titers. Likewise, it was unexpectedly found that among the patient pool tested, all patients with hepatitis B virus that contains a cysteine at aa256 instead of a serine at that position demonstrated a failure in long term lamivudine therapy, i.e., were diagnosed over time with a rebound in virus titer or no substantive change in virus titers.

The following HBV mutation is also highly correlated with the failure of a long-term response to lamivudine: Q213S (glutamine to serine at aa213) (originally codon 604) in the HBV polymerase region. In addition, the nucleotide changes G1739T, A1752C/T, T1909C, T1960G, and T1961A/G represent specific point mutations in the DNA precore/core promoter or open reading frame (ORF) region of HBV that are correlated with therapy failure. Further, the pairs of nucleotide changes A1738C and G1739T, A1750G and A1752G, T1909G and A1911T, and T1961A and C1962A represent specific double point mutations in the DNA precore/core promoter or open reading frame (ORF) region of HBV that are correlated with therapy failure.

Any one of these natural variations or mutations independently or in any combination can be used to predict the efficacy of long term lamivudine therapy.

This invention solves a long felt need particularly in those areas of the world with patients predominantly infected with HBeAg negative hepatitis B virus, and whose virus mutates to a lamivudine resistant strain and then breaks through to near pretreatment levels or higher. The diagnostic described herein allows a health care provider to determine in advance whether a patient will fail long term lamivudine therapy, and allows him or her to consider alternative treatment regimens or at a minimum, monitor the lamivudine treatment carefully. This represents a true advance in the art of anti-viral therapy.

In one embodiment, therefore, the invention provides materials, methods and kits for predicting the long-term response to therapy with lamivudine, of a chronic hepatitis B virus (HBV) carrier, and in particular a HBeAg negative hepatitis B virus carrier, that includes determining whether the patient carries a strain of hepatitis B that bears one or more correlative amino acids and/or nucleotides at the specified locations.

In another embodiment, the invention provides materials, methods and kits for detecting viral markers for predicting the long term response of an HBV carrier to therapy with lamivudine, wherein nucleic acids from a sample containing HBV nucleic acids are directly sequenced and the sequence is analyzed using methods of nucleic acid sequencing known to persons of ordinary skill in the art to determine whether such markers are present. Alternatively, the HBV nucleic acids contained in the sample can be amplified using standard nucleic acid amplification techniques prior to sequencing to determine whether the correlative viral markers are present. In addition, the HBV nucleic acid sequences contained in the sample can be cloned into any suitable vector known in the art using techniques known to persons of ordinary skill in the art prior to sequencing to determine whether the correlative viral markers are present.

In another embodiment, the invention provides materials, methods, and kits for detecting viral markers diagnostic for long term response of HBV carrier to 3TC therapy, wherein a sample containing HBV nucleic acid sequence is contacted with a oligonucleotide probe having a sequence complementary to a section of the gene that includes such viral markers; and the sequence is then detected by hybridization of the probe to the sequence. The hybridization of the probe to the viral marker correlated with therapy failure can be detected by labeling the probe using any detectable agent. The probe can be labeled for example, with a radioisotope, or with biotin, fluorescent dye, electron-dense reagent, enzyme, hapten or protein for which antibodies are available. The detectable label can be assayed by any desired means, including spectroscopic, photochemical, biochemical, immunochemical, radioisotopic, or chemical means. The probe can also be detected using techniques such as an oligomer restriction technique, a dot blot assay, a reverse dot blot assay, a line probe assay, and a 5′ nuclease assay. Alternatively, the probe can be detected using any of the generally applicable DNA array technologies, including macroarray, microarray and DNA microchip technologies.

In another embodiment, the invention provides materials, methods, and kits for detecting viral markers diagnostic for long term response of HBV carrier to 3TC therapy, wherein a sample containing HBV RNA is analyzed to determine if such HBV viral markers are present. Such analysis can be performed using any generally known RNA analysis technique in the art, including, but not limited to, RT-PCR, northern blot analysis, dot blot analysis, and nuclease protection assays (NPAs). Alternatively, the RNA probe can be detected using any of the generally applicable RNA array technologies, including macroarray, microarray and microchip technologies.

In another embodiment, the invention provides materials, methods, and kits for detecting viral markers diagnostic for long term response of HBV carrier to 3TC therapy, wherein a sample containing HBV proteins, peptides, or peptide fragments is analyzed for such HBV viral markers. The protein, peptide, or peptide fragment viral markers can be detected by any generally applicable protein, peptide, or peptide fragment detection technology known in the art, including western blot assays, two dimensional gel electrophoresis (2D-PAGE), enzyme linked immunosorbent assays (ELISA), enhanced chemiluminescence (ECL), immunohistochemistry, ELI-Spot assays, peptide sequencing, or antibody based protein array technology.

In still another embodiment, the invention provides materials, methods, and kits for detecting viral markers diagnostic for long term response of HBV carrier to 3TC therapy, wherein the amino acids from a sample containing HBV proteins, peptides, or peptide fragments are sequenced using methods known by persons of ordinary skill in the art to determine whether such viral markers are present.

In another embodiment, the invention provides materials, methods, and kits for detecting viral markers diagnostic for long term response of HBV carrier to 3TC therapy, wherein a sample containing HBV carrier antibodies is analyzed for reactivity to specific HBV proteins, peptides, or peptide fragments containing such viral markers. The HBV antibodies reactive to the specific protein, peptide, or peptide fragment viral markers can be detected by any generally applicable antibody based assays known in the art, including western blot, dot blot assays, two dimensional gel electrophoresis, enzyme linked immunosorbent assays (ELISA), enhanced chemiluminescence, and protein based array technology. (BCIP).

In an alternate embodiment, the invention provides a process for detecting a nucleic acid marker of the failure of long term lamivudine therapy for HBV infection, including the steps of amplifying the HBV nucleic acid sequence; contacting the amplified sequence with an oligonucleotide probe having a sequence complementary to a section of the gene that includes the viral marker of the HBV nucleic acid sequence; and then detecting the sequence by hybridizing the probe to the sequence. In one embodiment, amplification is achieved by use of the polymerase chain reaction method. The hybridization of the probe to the viral marker correlated with therapy failure can be detected by labeling the probe using any detectable agent. The probe can be labeled for example, with a radioisotope, or with biotin, fluorescent dye, electron-dense reagent, enzyme, hapten or protein for which antibodies are available. The detectable label can be assayed by any desired means, including spectroscopic, photochemical, biochemical, immunochemical, radioisotopic, or chemical means. The probe can also be detected using techniques such as an oligomer restriction technique, a dot blot assay, a reverse dot blot assay, a line probe assay, and a 5′ nuclease assay. Alternatively, the probe can be detected using any of the generally applicable DNA array technologies, including macroarray, microarray and DNA microchip technologies. In an alternate embodiment, the amplification can be achieved through any technique known in the art for such purposes, including, but not limited to, the ligase chain reaction, nucleic acid based sequence amplification, transcription-based amplification, or amplification by means of Q9 replicase. The hybridization of the probe to the viral marker correlated with therapy failure can be detected by labeling the probe using any detectable agent. The probe can be labeled for example, with a radioisotope, or with biotin, fluorescent dye, electron-dense reagent, enzyme, hapten or protein for which antibodies are available. The detectable label can be assayed by any desired means, including spectroscopic, photochemical, biochemical, immunochemical, radioisotopic, or chemical means. The probe can also be detected using techniques such as an oligomer restriction technique, a dot blot assay, a reverse dot blot assay, a line probe assay, and a 5′ nuclease assay. Alternatively, the probe can be detected using any of the generally applicable DNA array technologies, including macroarray, microarray and DNA microchip technologies.

In another embodiment, the invention provides oligonucleotide probes for the detection of the viral nucleic acid markers of long term lamivudine failure. In one embodiment, the oligonucleotide probes hybridize under sequence-hybridization conditions, including specific, stringent hybridization, to a nucleotide sequence that bears the marker and does not hybridize to sequences that do not contain the viral markers. In another embodiment, the oligonucleotides are at least 14 nucleotides in length. In an alternate embodiment, the oligonucleotides are at least 16, 18, 20, 25, or 28 nucleotides in length.

In an alternative embodiment, an antisense mimic is used to detect the correlative nucleic acid sequences, including a PNA (peptide nucleic acid), MNA (morpholinonucleic acid), LNA (locked nucleic acid), PCO (pseudocyclic oligonucleobase), or 2′-O,4′-C-ethylene bridged nucleic acid (ENA).

In specific embodiments, the oligonucleotide probe hybridizes to a nucleotide sequence that includes a codon that encodes for leucine at aa91 in the DNA polymerase region of HBV. In an alternate embodiment, the oligonucleotide probe hybridizes to a nucleotide sequence that includes a codon that encodes for cysteine at aa256 in the DNA polymerase region of HBV. In another embodiment, oligonucleotide probes that hybridize to sequences that include codons that encode for leucine at aa91 as well as cysteine at aa256 in the DNA polymerase region of HBV are used.

The oligonucleotide probe typically includes approximately at least 14, 15, 16, 18, 20, 25 or 28 nucleotides that hybridize to the nucleotides that include and are adjacent to those that encode for leucine at amino acid 91 in the DNA polymerase region of HBV. In a specific embodiment, the oligonucleotide probe can hybridize to one or more of the nucleotide sequences, Sequence ID Nos. 1-27, which encompass the nucleotides including and adjacent to amino acid 91. Further, the following additional codons can encode leucine at amino acid 91: TTA, TTG, CTC, CTA, or CTG. These codons can be substituted for the CTT sequence that is listed in Sequence ID Nos. 1-27. It is recognized that it is not necessary for the probe to be 100% complementary to the target nucleic acid sequence. As long as the probe can recognize the codon that encodes leucine at amino acid 91 a certain degree of base pair mismatch can generally be tolerated.

Alternatively or in addition, an oligonucleotide probe can be used that hybridizes to the nucleotides that include and are adjacent to the nucleotides that encode for cysteine at amino acid 256 in the polymerase region of HBV. In a specific embodiment, the oligonucleotide probe can hybridize to one or more of the nucleic acid sequences such as those in sequence ID Nos. 28-54, which encompass the nucleotides including and adjacent to amino acid 256 of the HBV polymerase. Further, the codon TGC can also encode cysteine at amino acid 256. This nucleotide triplet can be substituted for the TGT sequence that is listed in Sequence ID Nos. 28-54. Again, it is recognized that it is not necessary for the probe to be 100% complementary to the target nucleic acid sequence. As long as the probe can recognize the codon that encodes cysteine at amino acid 256, a certain degree of base pair mismatch can generally be tolerated.

In another embodiment, the oligonucleotide probe hybridizes to sequences that include codons that encode for serine at aa213 in the DNA polymerase region of HBV. In a specific embodiment, the oligonucleotide probes can hybridize to at least one of the nucleotide sequences in sequence ID Nos. 55-81, which encompass the nucleotides including and adjacent to amino acid 213 of the HBV polymerase. Further, the following additional codons can encode serine at amino acid 213: TCA, TCC, TCT, AGT, AGC, and TCG. These codons can be substituted for the TCG sequence that is listed in Sequence ID Nos. 55-81. It is recognized that it is not necessary for the probe to be 100% complementary to the target nucleic acid sequence. As long as the probe can recognize the codon that encodes serine at amino acid 213, a certain degree of base pair mismatch can generally be tolerated.

In another embodiment, the oligonucleotide probe hybridizes specifically to a nucleotide sequence that includes at least one nucleotide selected from the group consisting of: T1739; C/T1752; C1909; G1960; A1961; and G1961, which represent specific point mutations in the DNA precore/core promoter or open reading frame (ORF) region of HBV that are correlated with therapy failure. In specific embodiments, the oligonucleotide probes can hybridize to at least one of the nucleic acid sequences such as those in sequence ID Nos. 82-108; 109-135, 136-162; 163-189; 217-243; 190-216 which encompass the nucleotides including and adjacent to T1739; C/T1752; C1909; G1960; A1961; and G1961, respectively, of the DNA precore/core promoter or open reading frame (ORF) region of HBV.

In another embodiment, the oligonucleotide probe hybridizes specifically to a nucleotide sequence that includes at least one pair of nucleotides selected from the group consisting of: C1738 and T1739; G1750 and G1752; G1909 and T1911; A1961 and A1962; which represent specific double point mutations in the DNA precore/core promoter or open reading frame (ORF) region of HBV that are correlated with therapy failure. In specific embodiments, the oligonucleotide probe can hybridize to at least one of the nucleic acid sequences such as those in sequence ID Nos. 244-270, 271-297, 298-324, and 325-351 which encompass the nucleotides including and adjacent to C1738 and T1739; G1750 and G1752; G1909 and T1911; A1961 and A1962, respectively, of the DNA precore/core promoter or open reading frame (ORF) region of HBV.

In another embodiment, the invention provides an oligonucleotide primer for amplifying an HBV nucleic acid sequence. In one embodiment, the oligonucleotide is at least 14 nucleotides in length and hybridizes under sequence-specific, stringent hybridization conditions to a nucleotide sequence that distinguishes the HBV containing the nucleotide viral markers for lamivudine failure from HBV that does not contain the viral markers.

In another embodiment, the invention provides a kit for the detection of markers of resistance to long term lamivudine treatment of hepatitis B virus, and in particular, HbeAg negative virus. The kit can contain a compartment which contains an oligonucleotide probe which binds substantially to a nucleic acid subsequence of the HBV that contains the marker nucleic acid viral marker. The kit can also contain reagents to detect the hybridization of the probe to the HBV nucleic acid viral marker. The present invention can also include kits that can contain primers for the PCR amplification of HBV nucleic acids. A kit can also contain means for detecting amplified HBV nucleic acids, such as an oligonucleotide probe. In some cases, the probe is fixed to an appropriate support membrane. Other optional components of the kit include, for example, an agent to catalyze the synthesis of primer extension products, the substrate nucleoside triphosphates, means used to label (for example, an avidin-enzyme conjugate and enzyme substrate and chromogen if the label is biotin), the appropriate buffers for PCR or hybridization reactions, and instructions for carrying out the present method.

Additionally, the invention provides a method, materials and a kit to detect proteins, peptides or peptide fragments that contain amino acids (as described extensively herein) that are predictive of the long term response of an HBV carrier to 3TC therapy, or antibodies to those proteins, peptides or peptide fragments. Host sera or tissue can be tested for either the protein or peptide or the antibody to the protein or peptide, depending on convenience and perhaps concentration of the diagnostic material.

The protein, peptide or peptide fragment can be confirmed by reaction with an antibody, preferably a monoclonal antibody, for example using a Western blot method. Alternatively, the protein or peptide can be isolated and sequenced or otherwise identified by any means known in the art, including by 2D PAGE. In specific embodiments, a reactive antibody binds to an HBV protein or peptide sequence that includes an amino acid that encodes for leucine at aa91 in the DNA polymerase region of HBV. In an alternate embodiment, the reactive antibody binds to an HBV peptide or protein sequence that includes an amino acid sequence that encodes for cysteine at aa256 in the DNA polymerase region of HBV. In another embodiment, the reactive antibody binds to HBV proteins or peptides that include an amino acid for serine at aa213.

In another embodiment, the reactive antibody binds specifically to a peptide sequence that includes an amino acid that is substituted by mutation of a nucleotide selected from the group consisting of: T1739; C/T1752; C1909; G1960; A1961; and G1961, which represent specific point mutations in the DNA precore/core promoter or open reading frame (ORF) region of HBV that are correlated with therapy failure.

In specific embodiments, an antibody is used that binds to at least one peptide or peptide fragment encoded for by the nucleic acid sequences in sequence ID Nos. 82-216.

In another embodiment, the reactive antibody binds specifically to a peptide sequence that includes an amino acid that is substituted by mutation of a nucleotide selected from the group consisting of: C1738 and T1739; G1750 and G1752; G1909 and T1911; and A1961 and A1962; which represent specific double point mutations in the DNA precore/core promoter or open reading frame (ORF) region of HBV that are correlated with therapy failure.

In specific embodiments, an antibody is used that binds to at least one peptide or peptide fragment encoded for by the nucleic acid sequences in sequence ID Nos. 271-351.

### BRIEF DESCRIPTION OF THE DRAWINGS

FIG. 1 depicts the locations of the major sequences associated with lamivudine treatment outcome. Panel A depicts one nonlimiting example of domains A to E of the HBV polymerase (amino acids 410 to 592 for genotype D). The amino acid positions for the HBV polymerase gene are numbered to be consistent with the newly established scheme designed to standardize the nomenclature for lamivudine-resistance mutations, L180M and M204V/I (originally designated as L528M or L526M, and M552V/I or M550V/I) (Stuyver, et al., Hepatology 33:751-757 (2001)). To avoid confusion, the original amino acid numbering scheme (Allen, et al., Hepatology 27:1670-1677 (1998); Pillay, et al., International Antiviral News 6(9):167-169 (1998)) is also noted as several previously described mutations discussed used that nomenclature. The numbering used here is most specific for the consensus sequence for genotype D. There are analogous sequences in other genotypes, or in variation of genotype D that may differ by a few nucleotides, however, the correlation among genotypes and variations will be obvious to one of ordinary skill in the art using conventional alignment approaches. Panel B depicts another nonlimiting example of the HBV precore/core region sequence. The start/stop sites of the overlapping genes and the corresponding location of the HBV epsilon (ε) step-loop RNA structure are shown for reference.

FIGS. 2-27 are schematic illustration of target nucleic acid sequence which contains a diagnostic nucleotide codon/nucleic acid(s) ZZZ. Alphabetic letters are used independently to symbolize nucleotide bases. These Figures are read in conjunction with Tables 1-13, which illustrate nucleic acid sequences encompassing codons or nucleic acids that are diagnostic for a long term response to 3TC therapy. Each nucleic acid sequence in Tables 1-13 is a 28mer. However, according to the invention any nucleic acid sequence from 14-28 or 30 bases in length can be used in the diagnostic process, material and kit. Therefore, FIGS. 2-27 are intended to constitute specific disclosures of each 14 through 28mer of each nucleic acid sequence in Tables 1-13. Complementary probes to the target sequence are also intended to be likewise specifically depicted by these Figures. FIG. 2 specifically discloses 14mers through 28mers of Seq ID Nos 2, 29, 56, 83, 110, 137, 164, 191, 218, 245, 272, 299, or 326. FIG. 3 specifically discloses 14mers through 28mers of Seq IDs 3, 30, 57, 84, 111, 138, 165, 192, 219, 246, 273, 300, or 327. FIG. 4 specifically discloses 14mers through 28mers of Seq IDs 4, 31, 58, 95, 112, 139, 166, 193, 220, 247, 301, or 328. FIG. 5 specifically discloses 14mers through 28mers of Seq IDs 5, 32, 59, 86, 113, 140, 167, 194, 221, 248, 275, 302, or 329. FIG. 6 specifically discloses 14mers through 28mers of Seq IDs 6, 33, 60, 87, 114, 141, 168, 195, 222, 249, 276, 303, or 330. FIG. 7 specifically discloses 14mers through 28mers of Seq IDs 7, 34, 61, 88, 115, 142, 169, 196, 223, 250, 277, 304, or 331. FIG. 8 specifically discloses 14mers through 28mers of Seq IDs 8, 35, 62, 89, 116, 143, 170, 197, 224, 251, 278, 305, or 332. FIG. 9 specifically discloses 14mers through 28mers of Seq IDs 9, 36, 63, 90, 117, 144, 171, 198, 225, 252, 279, 306, or 333. FIG. 10 specifically discloses 14mers through 28mers of Seq IDs 10, 37, 64, 91, 118, 145, 172, 199, 226, 253, 280, 307, or 334. FIG. 11 specifically discloses 14mers through 28mers of Seq IDs 11, 38, 65, 92, 119, 146, 173, 200, 227, 254, 281, 308, or 335. FIG. 12 specifically discloses 14mers through 28mers of Seq IDs 12, 39, 66, 83, 120, 147, 174, 201, 228, 255, 282, 309, or 336. FIG. 13 specifically discloses 14mers through 28mers of Seq IDs 13, 40, 67, 84, 121, 148, 175, 202, 229, 256, 283, 310, or 337. FIG. 14 specifically discloses 15mers through 28mers of Seq IDs 14, 41, 68, 95, 122, 149, 176, 203, 230, 257, 284, 311, or 338. FIG. 15 specifically discloses 16mers through 28mers of Seq IDs 15, 42, 69, 96, 123, 150, 177, 204, 231, 258, 285, 312, or 339. FIG. 16 specifically discloses 17mers through 28mers of Seq IDs 16, 43, 70, 97, 124, 151, 178, 205, 232, 259, 286, 313, or 340. FIG. 17 specifically discloses 18mers through 28mers of Seq IDs 17, 44, 71, 98, 125, 152, 179, 206, 233, 260, 287, 314, or 341. FIG. 18 specifically discloses 19mers through 28mers of Seq IDs 18, 45, 72, 99, 126, 153, 180, 207, 234, 261, 288, 315, or 342. FIG. 19 specifically discloses 20mers through 28mers of Seq IDs 19, 46, 73, 100, 127, 154, 181, 208, 235, 262, 289, 316, or 343. FIG. 20 specifically discloses 21mers through 28mers of Seq IDs 20, 47, 74, 101, 128, 155, 182, 209, 236, 263, 290, 317, or 344. FIG. 21 specifically discloses 22mers through 28mers of Seq IDs 21, 48, 75, 102, 129, 156, 183, 210, 237, 264, 291, 318, or 345. FIG. 22 specifically discloses 23mers through 28mers of Seq IDs 22, 49, 76, 103, 130, 157, 184, 211, 238, 265, 292, 319, or 346. FIG. 23 specifically discloses 24mers through 28mers of Seq IDs 23, 50, 77, 104, 131, 158, 185, 212, 239, 266, 293, 320, or 347. FIG. 24 specifically discloses 25mers through 28mers of Seq IDs 24, 51, 78, 105, 132, 159, 186, 213, 240, 267, 294, 321, or 348. FIG. 25 specifically discloses 26mers through 28mers of Seq IDs 25, 52, 79, 106, 133, 160, 187, 214, 241, 268, 295, 322, or 349. FIG. 26 specifically discloses 27 and 28mers of Seq IDs 26, 53, 80, 107, 134, 161, 188, 215, 242, 269, 296, 323, or 350. FIG. 27 specifically discloses 28mers of Seq IDs 27, 54, 81, 108, 135, 162, 189, 216, 243, 270, 297, 324, or 351.

### DETAILED DESCRIPTION

The invention is a process, product, materials, and kit for predicting the long-term response of a chronic hepatitis B virus (HBV) carrier to therapy with lamivudine through the identification of specific vial markers. The invention describes oligonucleotide probes that bind to and/or detect the nucleic acid viral markers that predict the long term response of a HBV carrier to therapy with lamivudine. In addition, the invention describes amino acid probes that detect HBV viral markers that predict the long term responses of a HBV carrier to therapy with lamivudine. In one embodiment the viral marker used to predict the long-term response of a chronic hepatitis B virus (HBV) carrier to therapy with lamivudine is the presence of a specific amino acid at a certain position of the HBV. In one embodiment the viral marker used to predict the long-term response of a chronic hepatitis B virus (HBV) carrier to therapy with lamivudine is the presence of a leucine at aa91 (originally codon 438) of the HBV polymerase. In another embodiment the viral marker used to predict the long-term response of a chronic hepatitis B virus (HBV) carrier to therapy with lamivudine is the presence of a cysteine at aa256 (originally codon 604) of the HBV polymerase. In an alternate embodiment the viral markers used to predict the long-term response of a chronic hepatitis B virus (HBV) carrier to therapy with lamivudine is the presence of leucine at aa91 and cysteine at aa256 of the HBV polymerase. Other diagnostic markers that can predict the long-term response of a chronic hepatitis B virus (HBV) carrier to therapy with lamivudine are the following HBV mutations: Q213S in the HBV polymerase (originally codon 561); and nucleotide changes in the HBV precore/core regions G1739T; A1738C and G1739T; A1750G and A1752G; A1752C/r; T1909C; T1909G and A1911T; T1960G; T1961A/G; and T1961A and C1962A.

This invention solves a long felt need in those areas of the world with patients predominantly infected with HBeAg negative hepatitis B virus, particularly those patients whose virus mutates to a lamivudine resistant strain and then breaks through to wild type virus. The diagnostic described herein allows a health care provider to determine in advance whether an HBeAg negative patient will fail long term lamivudine therapy, and allows him or her to consider alternative treatment regimens or at a minimum, monitor the lamivudine treatment carefully. This represents a true advance in the art of anti-viral therapy.

Definitions

The following definitions and term construction are intended, unless otherwise indicated.

“Amplification reagents” refer to the various buffers, enzymes, primers, deoxynucleoside triphosphates (both conventional and unconventional), and primers used to perform the selected amplification procedure.

“Amplifying” or “Amplification”, which typically refers to an “exponential” increase in target nucleic acid, is being used herein to describe both linear and exponential increases in the numbers of a select target sequence of nucleic acid.

“Bind(s) substantially” refers to complementary hybridization between an oligonucleotide and a target sequence and embraces minor mismatches that can be accommodated by reducing the stringency of the hybridization media to achieve the desired priming for the PCR polymerases or detection of hybridization signal.

“Array” refers to any means that allows for the detection of the presence of targeted biomarkers in parallel through the use of an orderly arrangement of probes affixed to a solid support. An array analysis can make use of common assay systems such as microplates or, standard blotting membranes, and can be created by hand or can make use of robotics to deposit the sample. In general, arrays are described as macroarrays or microarrays, the difference being the size of the sample spots. Macroarrays contain sample spot sizes of about 300 microns or larger and can be easily imaged by existing gel and blot scanners. The sample spot sizes in microarray are typically less than 300 microns in diameter and these arrays can contain thousands of spots. In addition, arrays can be automated via robotic means.

“Microchip” refers to a microarray, typically fabricated by high-speed robotics, generally on glass but sometimes on nylon substrates, for which probes with known identity are used to determine complementary binding.

“Hybridizing” refers the binding of two single stranded nucleic acids via complementary base pairing.

“Nucleic acid” refers to a deoxyribonucleotide or ribonucleotide polymer in either single- or double-stranded form, and unless otherwise limited, would encompass known analogs of natural nucleotides that can function in a similar manner as naturally occurring nucleotides.

“Nucleotide polymerases” refers to enzymes able to catalyze the synthesis of DNA or RNA from nucleoside triphosphate precursors. In amplification reactions, the polymerases are template-dependent and typically add nucleotides to the 3′-end of the polymer being formed. The polymerase can be thermostable as described in U.S. Pat. Nos. 4,889,818 and 5,079,352.

The term “oligonucleotide” refers to a molecule comprised of two or more deoxyribonucleotides or ribonucleotides, such as primers, probes, nucleic acid fragments to be detected, and nucleic acid controls. The exact size of an oligonucleotide depends on many factors and the ultimate function or use of the oligonucleotide. Oligonucleotides can be prepared by any suitable method, including, for example, cloning and restriction of appropriate sequences and direct chemical synthesis by a method such as the phosphotriester method of Narang et al., Meth. Enzymol. 68:90-99 (1979); the phosphodiester method of Brown et al., Meth. Enzymol. 68:109-151 (1979); the diethylphosphoramidite method of Beaucage et al., Tetrahedron Lett. 22:1859-1862 (1981); and the solid support method of U.S. Pat. No. 4,458,066.

The term “primer” refers to an oligonucleotide, whether natural or synthetic, capable of acting as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. A primer is preferably a single-stranded oligodeoxyribonucleotide. The appropriate length of a primer depends on the intended use of the primer but typically ranges from 14 or 15 to 25 or 28 nucleotides. Short primer molecules generally require cooler temperatures to form sufficiently stable hybrid complexes with the template. A primer need not reflect the exact sequence of the template but must be sufficiently complementary to hybridize with a template.

The term “primer” can refer to more than one primer, particularly in the case where there is some ambiguity in the information regarding one or both ends of the target region to be amplified. For instance, if a region shows significant levels of polymorphism in a population, mixtures of primers can be prepared that will amplify alternate sequences. A primer can be labeled, if desired, by incorporating a label detectable by spectroscopic, photochemical, biochemical, immunochemical, or chemical means. For example, useful labels include ³²P, fluorescent dyes, electron-dense reagents, enzymes (as commonly used in an ELISA), biotin, or haptens and proteins for which antisera or monoclonal antibodies are available. A label can also be used to “capture” the primer, so as to facilitate the immobilization of either the primer or a primer extension product, such as amplified DNA, on a solid support.

“Probe” refers to an oligonucleotide which binds through complementary base pairing to a subsequence of a target nucleic acid. It will be understood by one of skill in the art that probes will typically substantially bind target sequences lacking complete complementarity with the probe sequence depending upon the stringency of the hybridization conditions. The probes are preferably directly labeled as with isotopes or indirectly labeled such as with biotin to which a streptavidin complex can later bind. By assaying for the presence or absence of the probe, one can detect the presence or absence of the target.

The terms “sequence-specific oligonucleotide” and “SSO” refer to oligonucleotides that have a sequence, called a “hybridizing region,” exactly complementary to the sequence to be detected, typically sequences characteristic of a particular allele or variant, which under “sequence-specific, stringent hybridization conditions” will hybridize only to that exact complementary target sequence. Relaxing the stringency of the hybridizing conditions will allow sequence mismatches to be tolerated; the degree of mismatch tolerated can be controlled by suitable adjustment of the hybridization conditions. Depending on the sequences being analyzed, one or more sequence-specific oligonucleotides can be employed.

As used herein, “sample” or “clinical sample” relates to any sample obtained from a host for use in carrying out the procedures of the present invention. In one aspect, the host is suffering from a disease or syndrome that is at least partially caused by a virus. The host may also be an asymptomatic considered to be at risk of viral infection. The sample may be a cellular sample such as a tissue sample, for example of lung tissue obtained as a biopsy or post-mortem, a fluid sample, such as blood, saliva, sputum, urine, cerebrospinal fluid, or a swabbed sample obtained by swabbing a mucus membrane surface such as nasal surface, a pharyngeal surface, a buccal surface, and the like, or it may be obtained from an excretion such as feces, or it may be obtained from other bodily tissues or body fluids commonly used in diagnostic testing.

“Subsequence” refers to a sequence of nucleic acids or amino acids that comprise a part of a longer sequence of nucleic acids or amino acids.

“Sequencing” refers to the determination of the ordered arrangement of a nucleic or amino acid using standard techniques known in the art by persons of ordinary skill.

The term “target region” refers to a region of a nucleic acid to be analyzed and can include a polymorphic region.

I. Detection of HBV Nucleic Acid Viral Markers

One aspect of the invention is a method for detecting the presence of HBV nucleic acid viral markers.

In one embodiment, the invention provides materials, methods, and kits for detecting a HBV viral marker for long term response to lamivudine therapy that includes directly sequencing the HBV nucleic acids from an HBV containing sample to determine if such viral markers are present in the sample.

In another embodiment, the invention provides materials, methods, and kits for detecting a marker of long term lamivudine resistance that includes contacting a sample containing an HBV nucleic acid sequence with an oligonucleotide probe having a sequence complementary to a section of the HBV genome that includes the marker; and then determining if the oligonucleotide hybridizes to the viral nucleic acid.

In another embodiment, the invention provides a process for detecting the presence of a marker of long term lamivudine resistance that includes:
(a) contacting the sample with an oligonucleotide probe that includes a sequence at least 14 nucleotides in length and is complementary to a section of HBV nucleic acid that contains the marker, under sequence-specific, stringent hybridization conditions; and
(b) detecting whether the hybridization of the probe occurs.

A. Direct Sequencing of Viral Nucleic Acids

Samples suspected of containing HBV nucleic acid sequences can be directly sequenced to detect the presence of viral markers for long term response to lamivudine therapy, such as those sequences depicted in Tables 1-13.

Generally, the method of the present embodiment involves extraction of the sample HBV nucleic acids, amplification of the viral nucleic acid sequence, direct sequencing of the products, and analysis of the direct sequence information using techniques known to a person of ordinary skill in the art (see, for example, The PCR Technique, eds. Ellingboe and Gyllensten, Eaton Publishing Company/Bio Techniques Books Division (1992)).

Amplification can be achieved via polymerase chain reaction (PCR; Saiki et al., 1988, see below), ligase chain reaction (LCR; Landgren et al., Science 241:1077-1080 (1988); Wu &Wallace, Genomics 4:560-569 (1989); Barany, Proc Natl Acad Sci USA 88:189-193 (1991)), nucleic acid sequence-based amplification (NASBA; Guatelli et al., Proc Natl Acad Sci USA 87:1874-1878 (1990); Compton, Nature 350:91-92 (1991)), transcription-based amplification system (TAS; Kwoh et al., Proc Natl Acad Sci USA 86:1173-1177. (1989)), strand displacement amplification (SDA; Duck, Biotechniques 9:142-147 (1990); Walker et al., Proc. Natl. Acad. Sci. USA 89, 392-396 (1992)), amplification by means of Q9 replicase (Lizardi et al., Nat. Genet. 19:225-232 (1998); Lomeli et al., Clin Chem 35:1826-1831 (1989)) or any other suitable method to amplify nucleic acid molecules known in the art. Amplification of the HBV nucleic acids can be accomplished using oligonucleotide primers with specific characteristics, such as, for example, those described herein complimentary to Seq IDs 1-351.

Direct sequencing of the nucleic acid sequence can be conducted by the chemical cleavage method (Maxam-Gilbert method), the chain terminator method (Sanger method, also known as the dideoxy method) or any other technique known to one skilled in the art (see also, Voet & Voet “Biochemistry: Second Edition”, Wiley & Sons, 1995, Chapter 28).

The sequences can then be analyzed to determine whether viral marker nucleic acid sequences are present in the sample, such as those sequences depicted in Tables 1-13.

If the sample contains viral RNA, cDNA can first be generated from HBV mRNA or total RNA through a reverse transcription technique known to those of ordinary skill in the art (see, for example, Paul Siebert, The PCR Technique, Eaton Publishing Company/Bio Techniques Books Division).

B. Detection Via Oligonucleotide Probes

1. Oligonucleotide Probes

Oligonucleotide probes are provided that are capable of detecting the presence of HBV nucleic acid markers of failure on long term lamivudine therapy. The probes are complementary to sequences of viral nucleic acids that include the markers. These probes can be used in processes and kits. The oligonucleotide probes can detect nucleotides that encode for leucine at aa91 (originally codon 438) and/or cysteine at aa256 (originally codon 604), which are sequence patterns in the DNA polymerase region of HBV that are correlated with therapy failure. In addition, the oligonucleotide probe can detect nucleotides that include codons that encode for serine at aa213 (originally codon 561), a mutation in the DNA polymerase region of HBV that is correlated with therapy failure. Alternatively, or in addition, an oligonucleotide probe is selected that can detect one of the following nucleotides: T1739; C1752; T1752; C1909; G1960; A1961; and G1961, which represent specific point mutations in the DNA precore/core promoter or open reading frame (ORF) region of HBV that are correlated with therapy failure. An oligonucleotide probe can, alternatively be used that can detect at least one pair of nucleotides selected from the group consisting of: C1738 and T1739; G1750 and G1752; G1909 and T1911; A1961 and A1962; A1961; and G1961, which represent specific double point mutations in the DNA precore/core promoter or open reading frame (ORF) region of HBV that are correlated with therapy failure.

The oligonucleotide probes are at least 14 nucleotides in length, and in a preferred embodiment, are at least 15, 16, 18, 20, 25 or 28 nucleotides in length. It is generally not preferred to use a probe that is greater than approximately 25 or 28 nucleotides in length. The oligonucleotide probe is designed to identify an HBV nucleotide sequence that contains a nucleotide(s) which is highly correlated with lamivudine treatment failure (the “diagnostic nucleotide(s)”). The oligonucleotide probe can be designed such that the diagnostic nucleotide(s) is in the interior section of the hybridized segment, or alternatively can be on either the 3′ or 5′ end of the hybridized segment. It is preferred that the diagnostic nucleotide(s) be located near the middle of the probe to allow efficient hybridization.

Tables 1-13 below are illustrative embodiments of HBV nucleotide sequences that include diagnostic nucleotides. Given these sequences, one of ordinary skill using standard algorithms can construct oligonucleotide probes that are complementary to the nucleotide sequences below. The rules for complementary pairing are well known: cytosine (“C”) always pairs with guanine (“G”) and thymine (“T”) or uracil (“U”) always pairs with adenine (“A”). It is recognized that it is not necessary for the probe to be 100% complementary to the target nucleic; acid sequence, as long as the probe sufficiently hybridizes and can recognize the diagnostic nucleotide. A certain degree of base pair mismatch can generally be tolerated. Therefore, in one embodiment, the oligonucleotide has 1, 2, 3, 4, 5 or 6 mismatches in complementarity to the HBV nucleotide sequence.

Furthermore, the corresponding amino acid sequences of the HBV viral markers diagnostic for long term response of HBV carriers to 3TC therapy can be determined from the illustrative HBV nucleotide sequences described in tables 1-13. Given these nucleotide sequences, one of ordinary skill using standard methods can construct peptides or peptide fragments that are complementary to the nucleotide sequences below. Technology for synthesis of peptides has been developed from classical methods applied for synthesis carried out in a solution (a survey of this technology is found in Houben-Weyl, Methoden der Organischen Chemie, Synthese yon Peptiden, E. Wunsch ed., Thieme, Berlin (1974)) through the synthesis technique developed by Merrifield applying a solid carrier in the form of particles (see, e.g., Stewart, J. M., and Young, J. D., Solid Phase Peptide Synthesis, Freeman, San Francisco (1985)). This technique has been found suitable for automation. See, e.g., Merrifield, R. B., Stewart, J. M., and Jernberg, N., Apparatus for the Automated Synthesis of Peptides, U.S. Pat. No. 3,531,258; Brunfeldt, K., Roepstorff, P., and Halstrom, J.; Reactions System, U.S. Pat. No. 3,577,077; Kubodera, J., Hara, T.; and Makabe, H., Apparatus for Synthesis of Peptides or the Like Organic Compounds, U.S. Pat. No. 3,647,390; Won Kil Park and Regoli, D., System for the Solid Phase Synthesis, U.S. Pat. No. 3,715,190; Bridgham, J., et al., Automated Peptide Synthesis Apparatus, U.S. Pat. No. 4,668,490. Such techniques are suitable for parallel synthesis of many peptides. See, e.g., Verbander, H. S., Fuller, W. D., and Goodman M., Rapid, Large Scale, Automatable High Pressure Peptide Synthesis, U.S. Pat. No. 4,192,798; Neimark, J., and Brand, J. P., Semi-Automatic, Solid-Phase Peptide Multi-Synthesizer and Process for the Production of Synthetic Peptides by the Use of Multi-Synthesizer and Process for the Production of Synthetic Peptides by the Use of Multi-Synthesizer, U.S. Pat. No. 4,748,002; Houghten, R. A., Means for Sequential Solid-Phase Organic Synthesis and Methods Using the Same, European Patent Application Publication No. 196,174 published Jan. 10, 1986; Geysen, H. M., Meloen, R. H., and Bartcling, S. J., Proc. Natl. Acad. Sci. U.S.A., Vol. 81, Page 3998 (1984); Frank, R., and Doring, R., “Simultaneous Multiple Peptide Synthesis under Continuous Flow Conditions on Cellulose Paper Discs as Segmental Solid Supports,” Tetrahedron, Vol. 44, No. 19, page 6031 (1988); Eichler, J., Beyermann, M., and Bienert, M., “Application of Cellulose Paper as Support Material in Simultaneous Solid Phase Peptide Synthesis” Collect. Czech. Chem. Commun., Vol 54, page 1746 (1989); Krchnak, V., Vagner, J., and Mach, O., “Multiple ContinuousFlow Solid-Phase Peptide Synthesis,” Int. J. Peptide Protein Res., Vol 33, page 209 (1989). The application of planar continuous carriers made it possible to carry out the so-called continuous synthesis of peptides. See Lebl M., Gut, V., Eichler, J., Krchnak, V. Vagner, J., and Stepanek, J., Method of a Continuous Peptide Synthesis on a Solid Carrier, Czechoslovak Patent Application No. PV 1280-89, to which European Patent Application Publication No. 385,433 published Sep. 5, 1990, corresponds.

In addition, while the Seq IDs represented in Tables 1-13 are illustrative of oligonucleotide probes 28 nucleotides in length, probes can be generated that are more than or less than the 28 nucleotides in order to identify HBV markers that are diagnostic for long term responses of HBV carriers to 3TC therapy. Tables 1-13 are illustrative of sequences flanking the diagnostic nucleotide that can be used to generate diagnostic probes. The oligonucleotide probes can be designed such that the diagnostic nucleotide(s) is in the interior section of the hybridized segment, or alternatively can be on either the 3′ or 5′ end of the hybridized segment. It is preferred that the diagnostic nucleotide(s) be located near the middle of the probe to allow efficient hybridization.

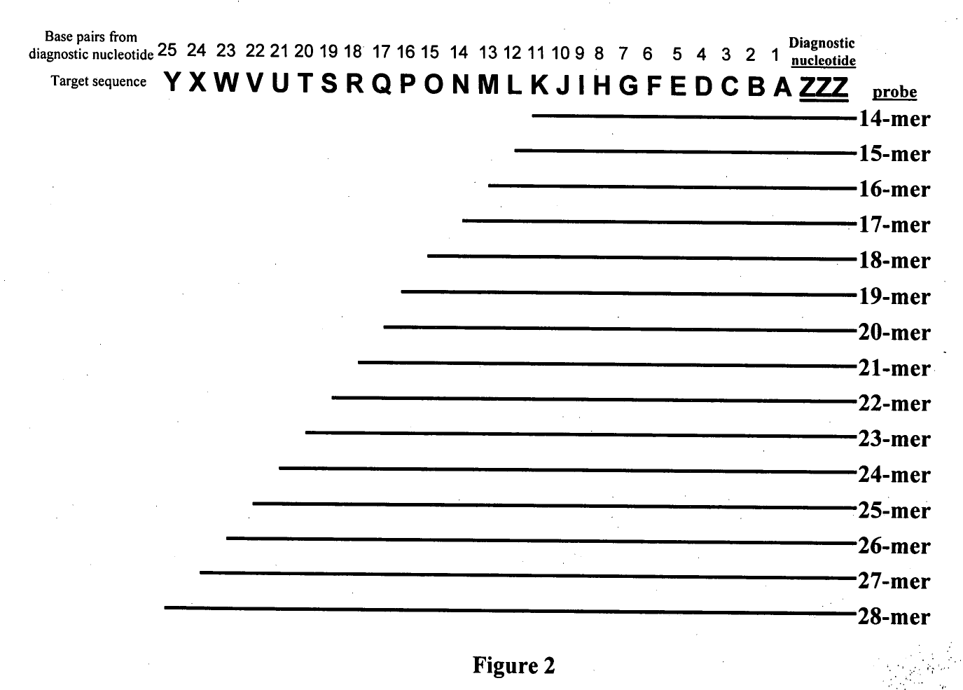

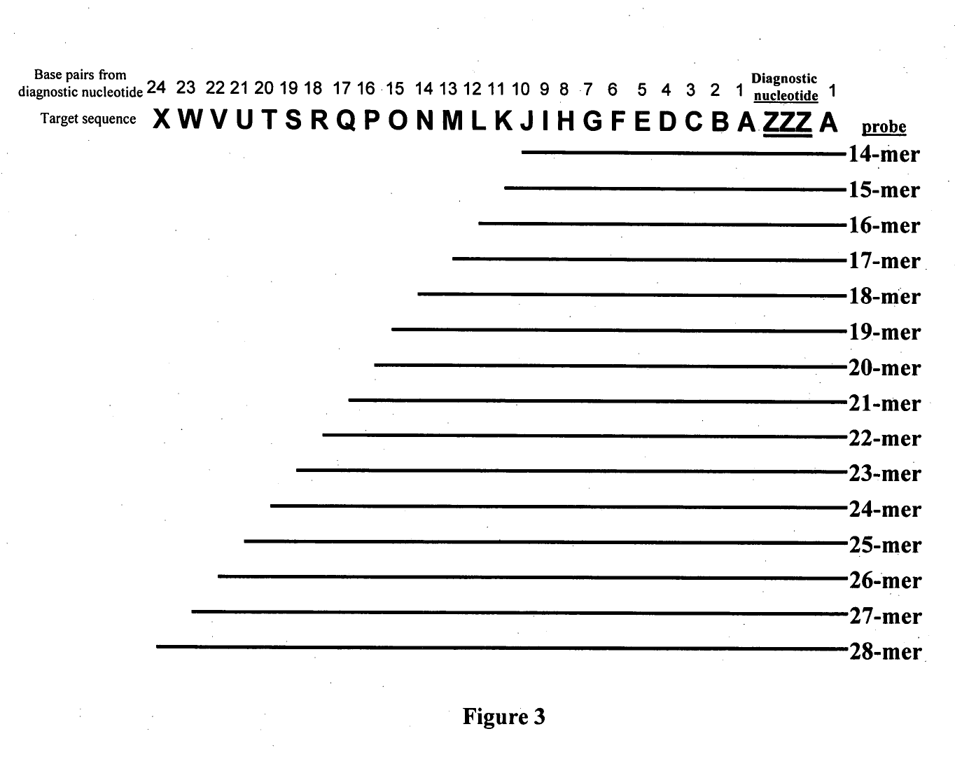

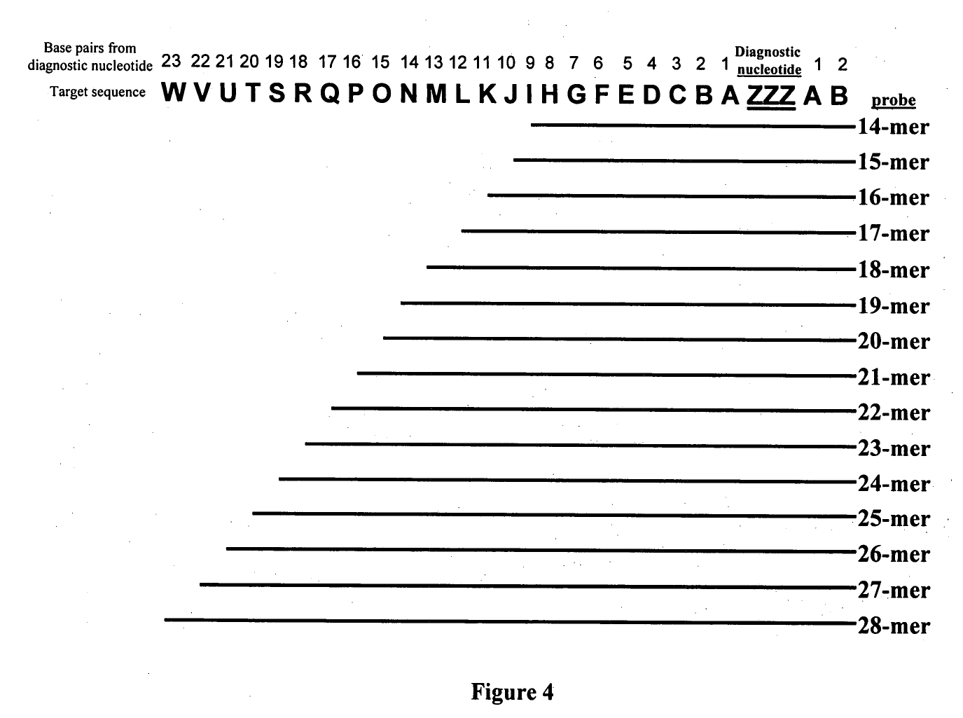

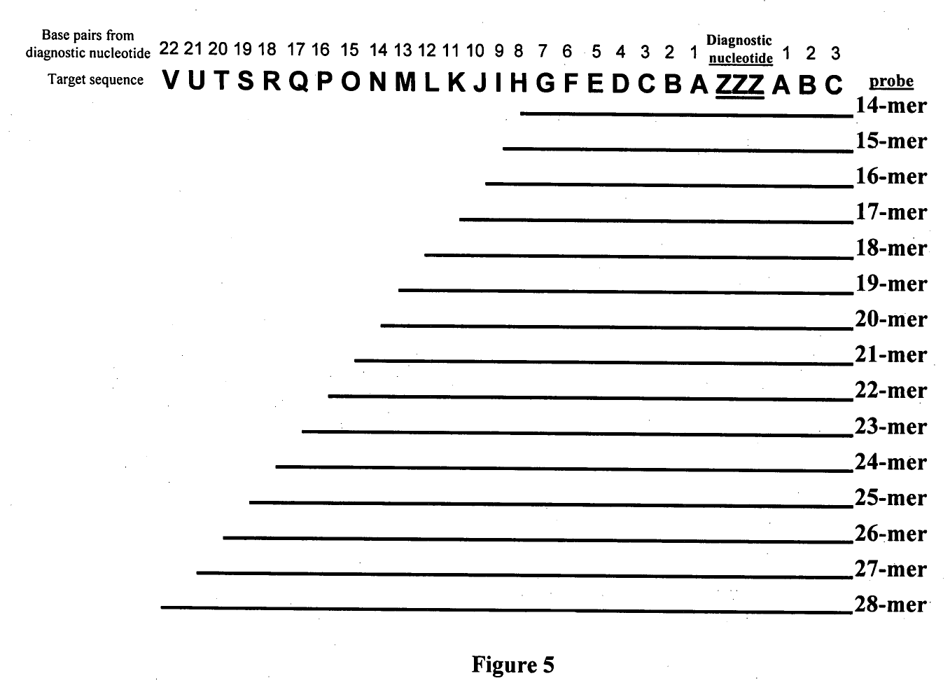

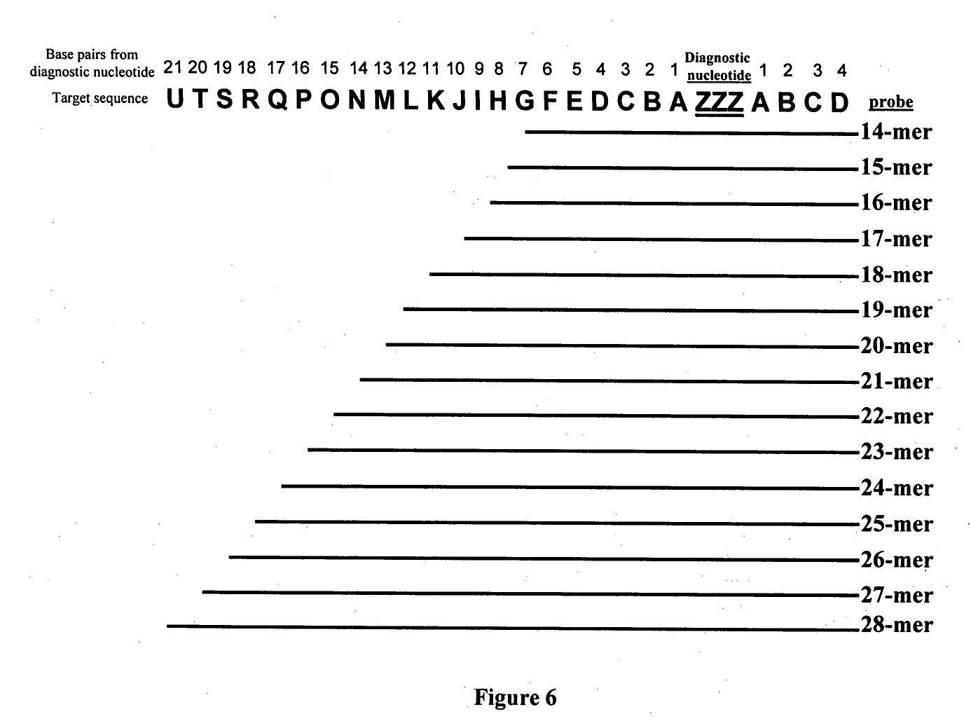

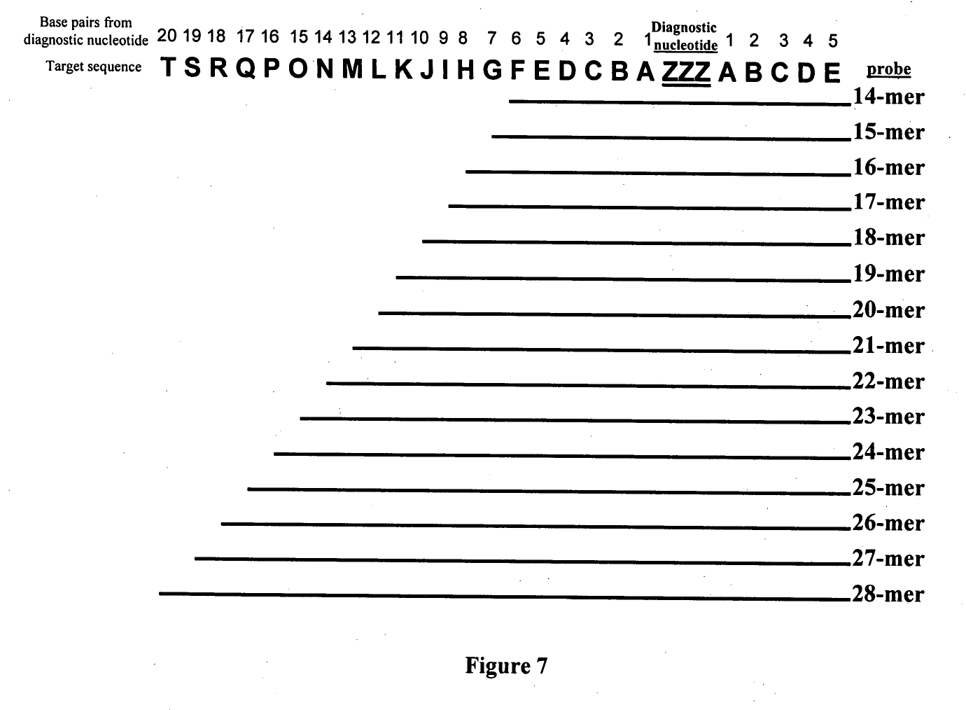

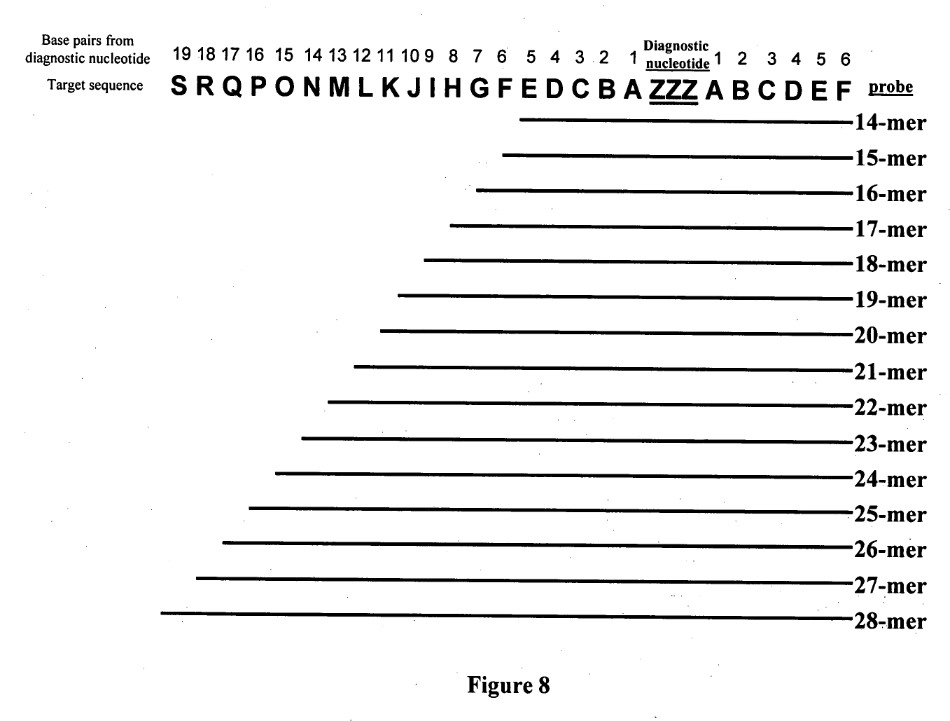

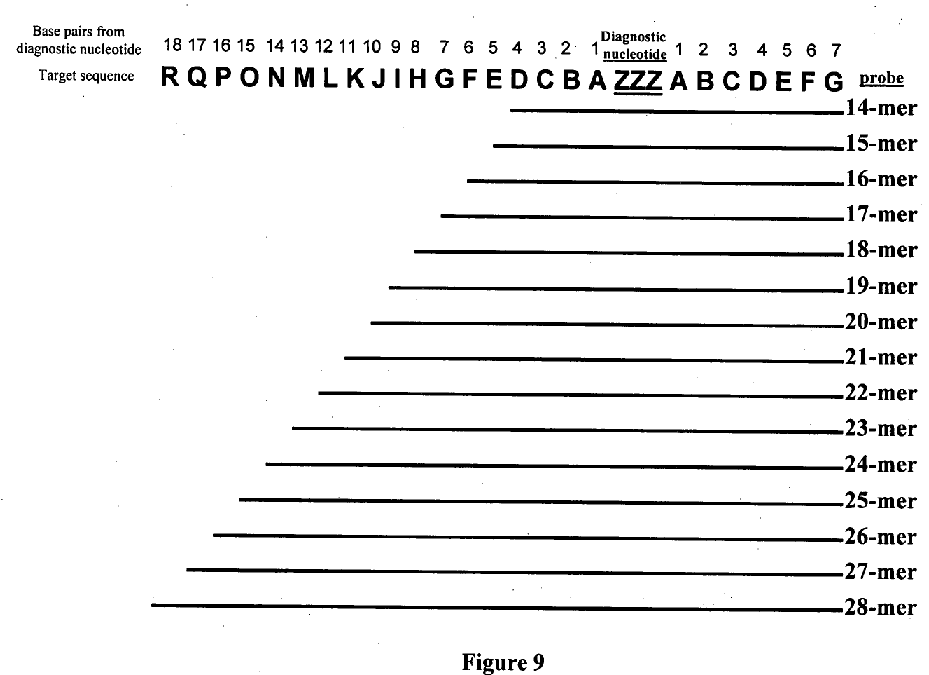

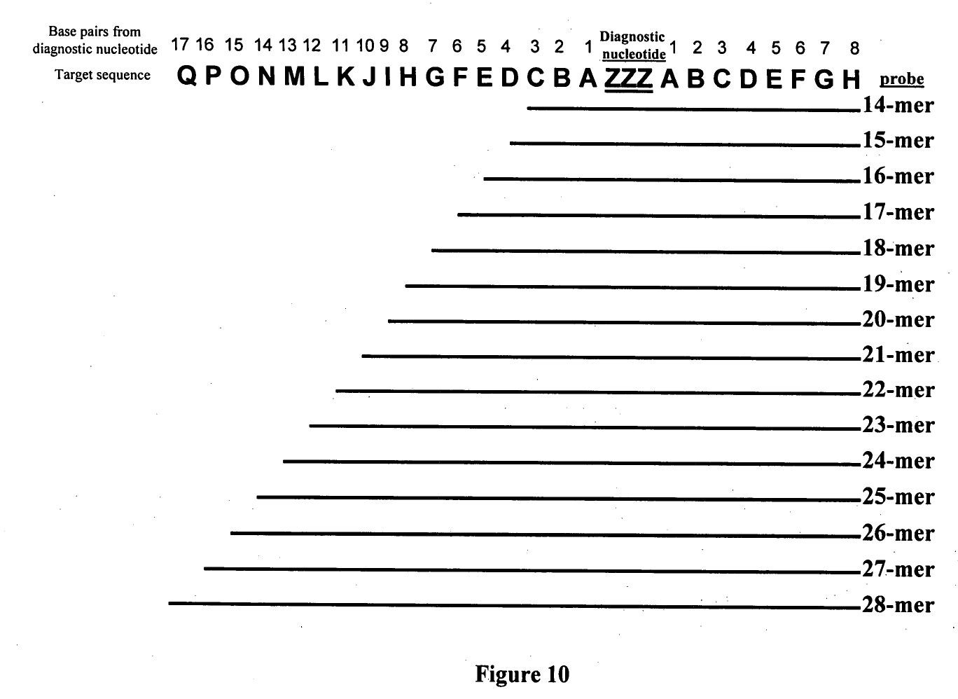

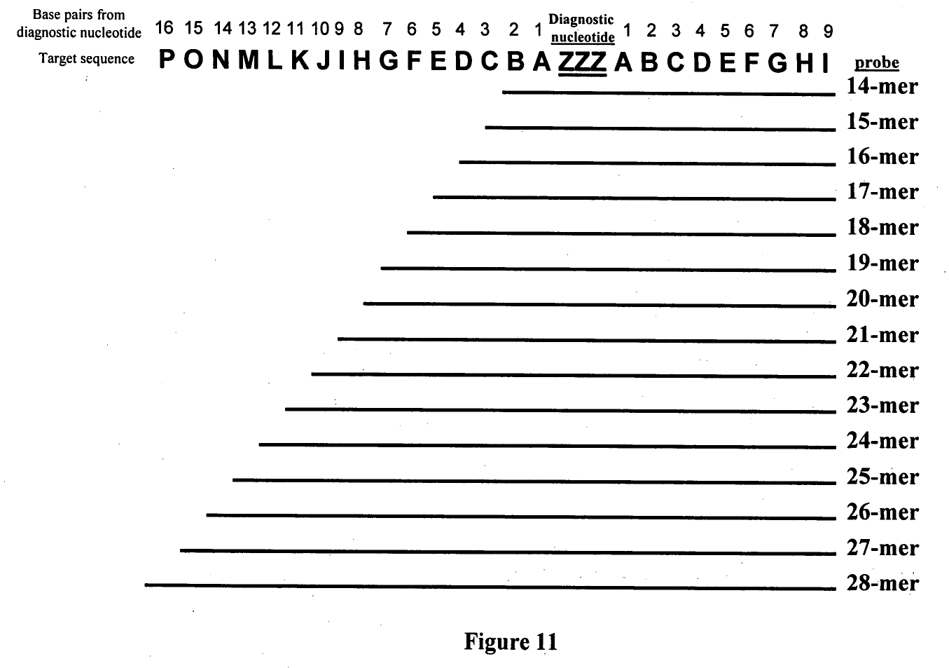

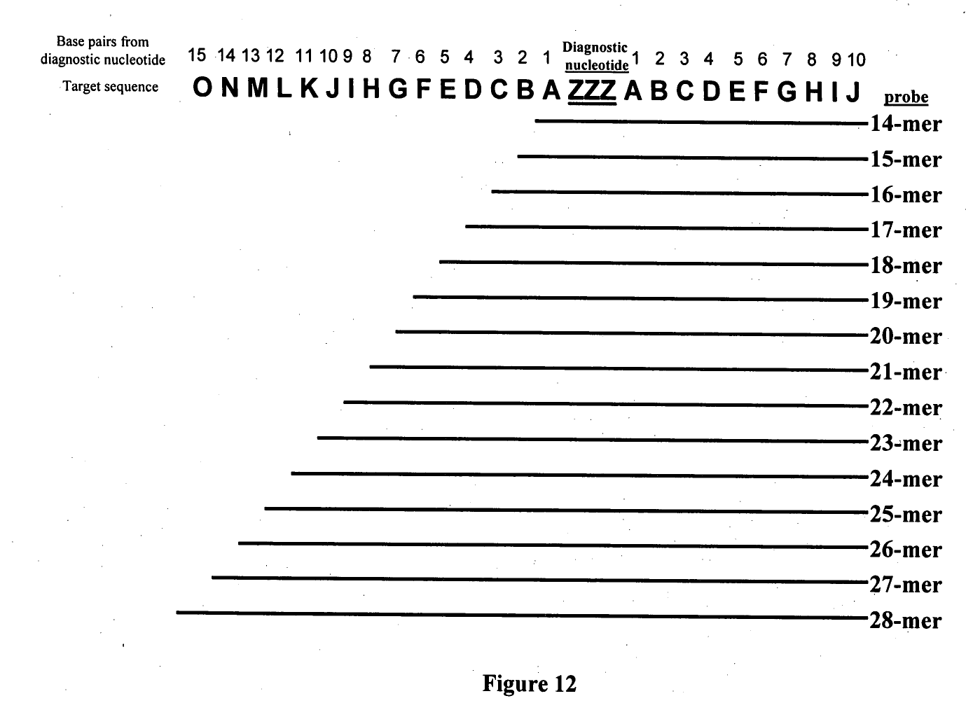

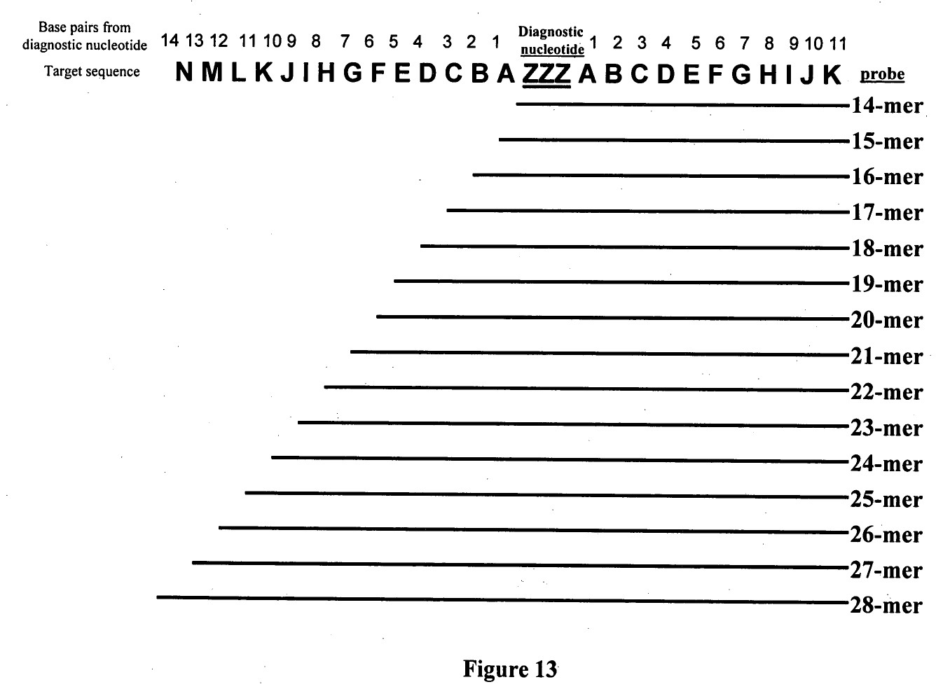

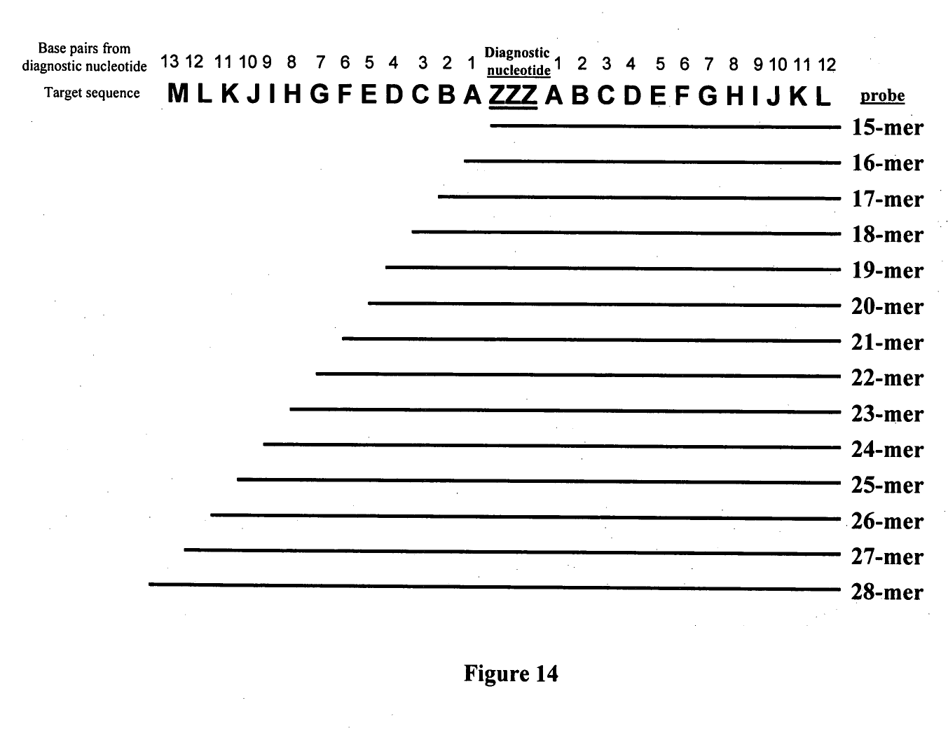

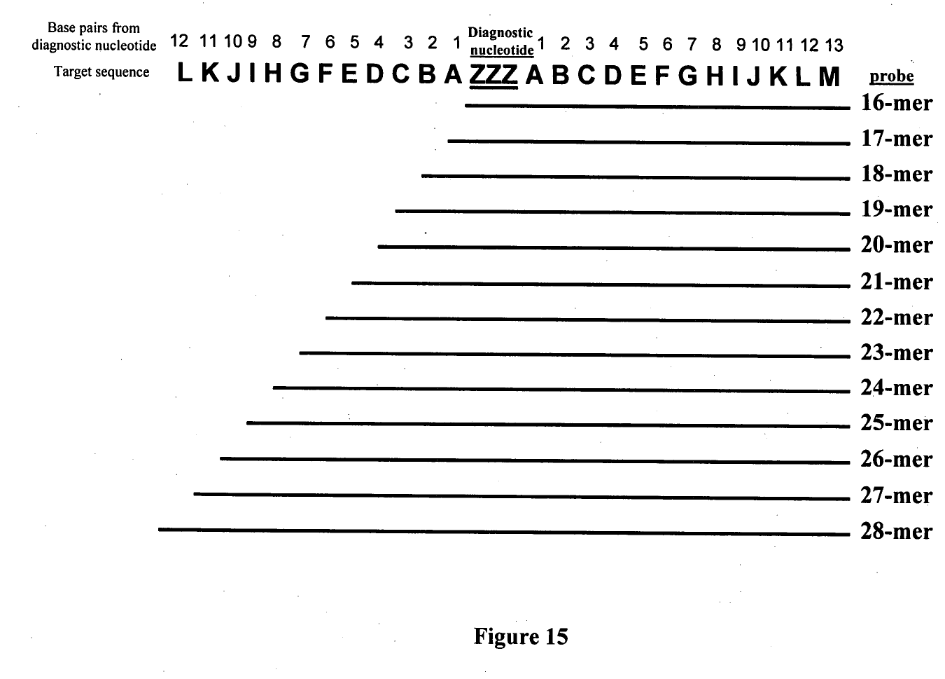

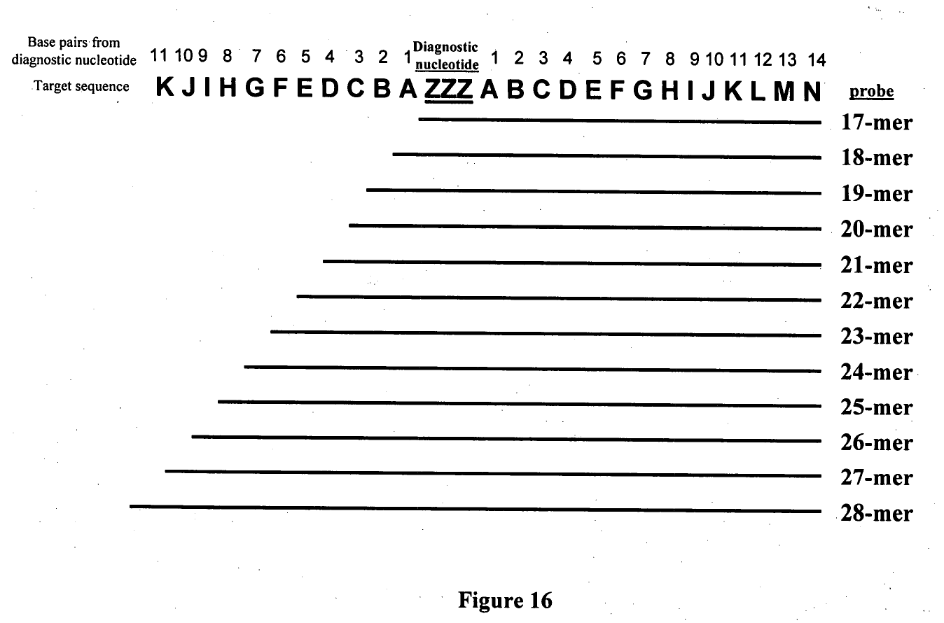

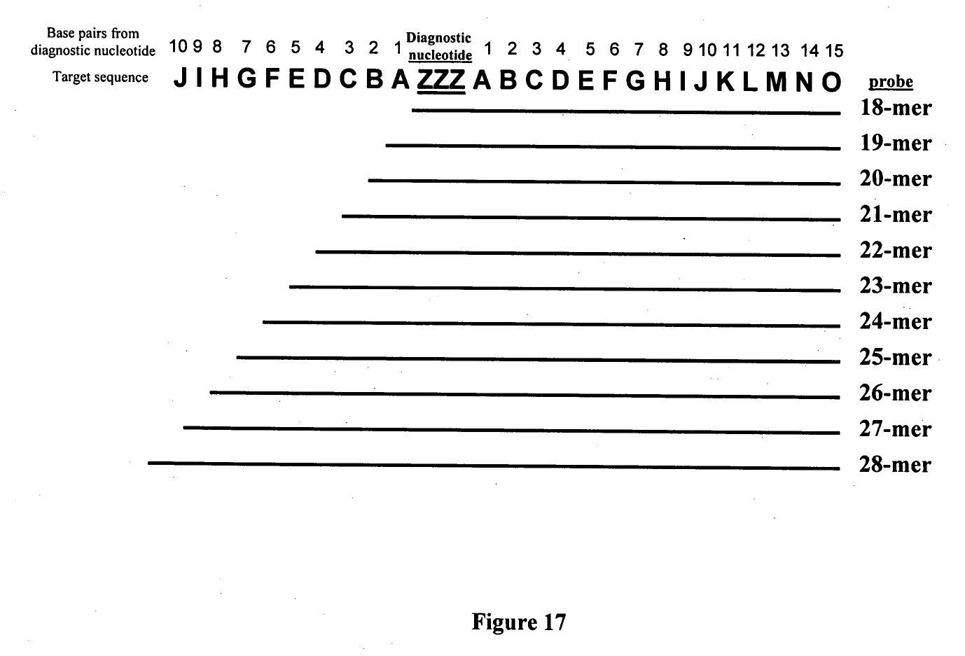

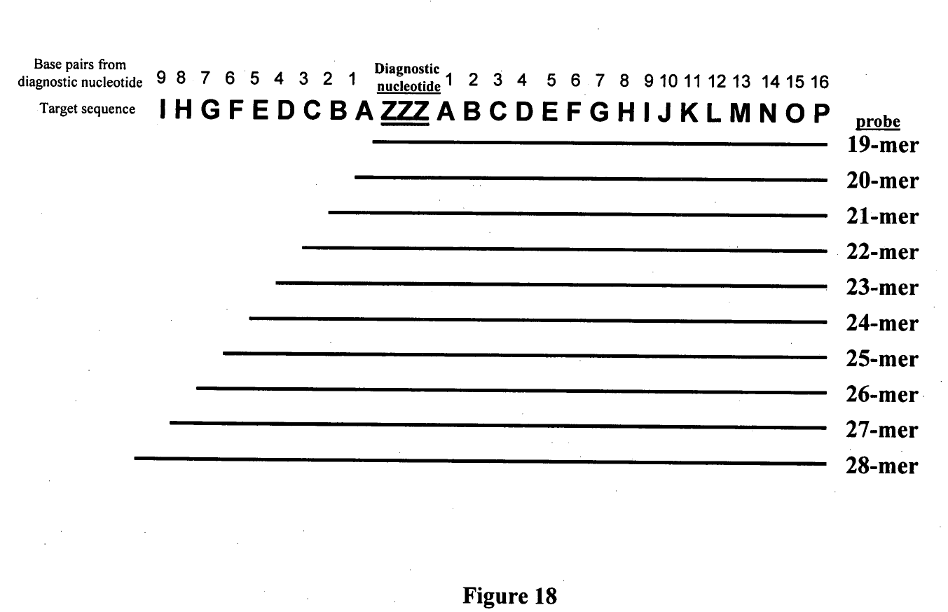

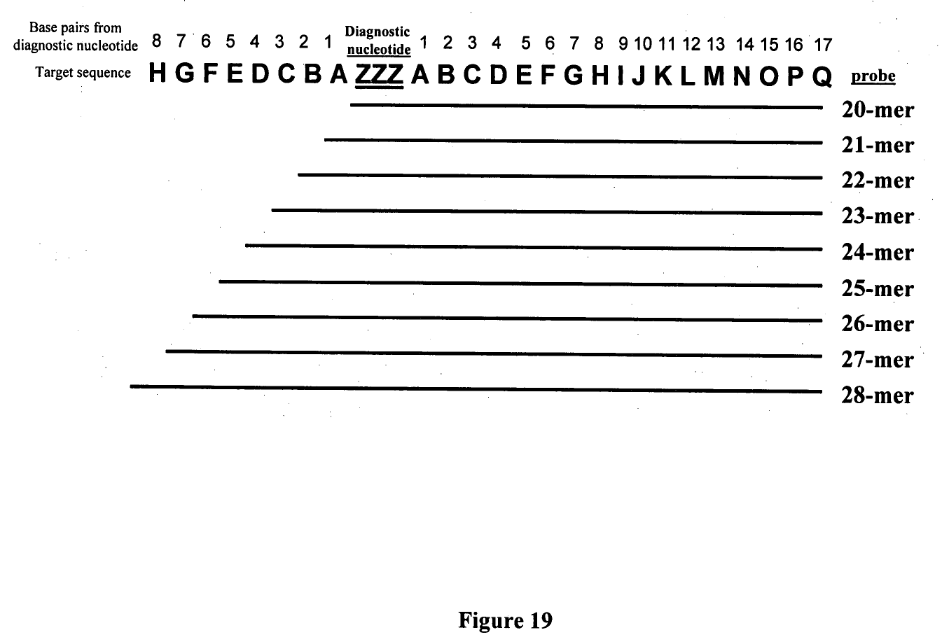

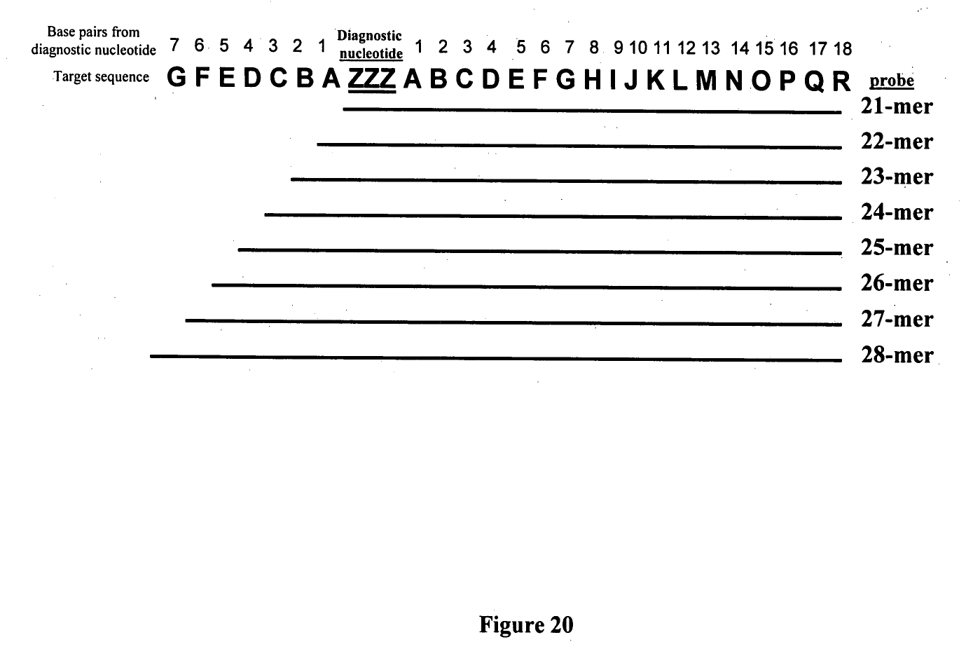

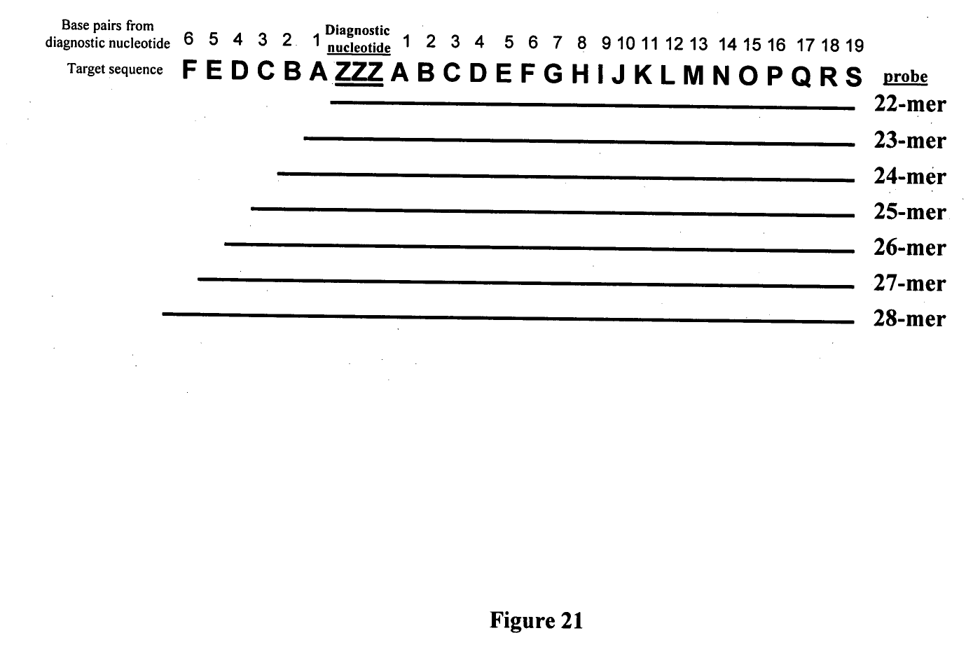

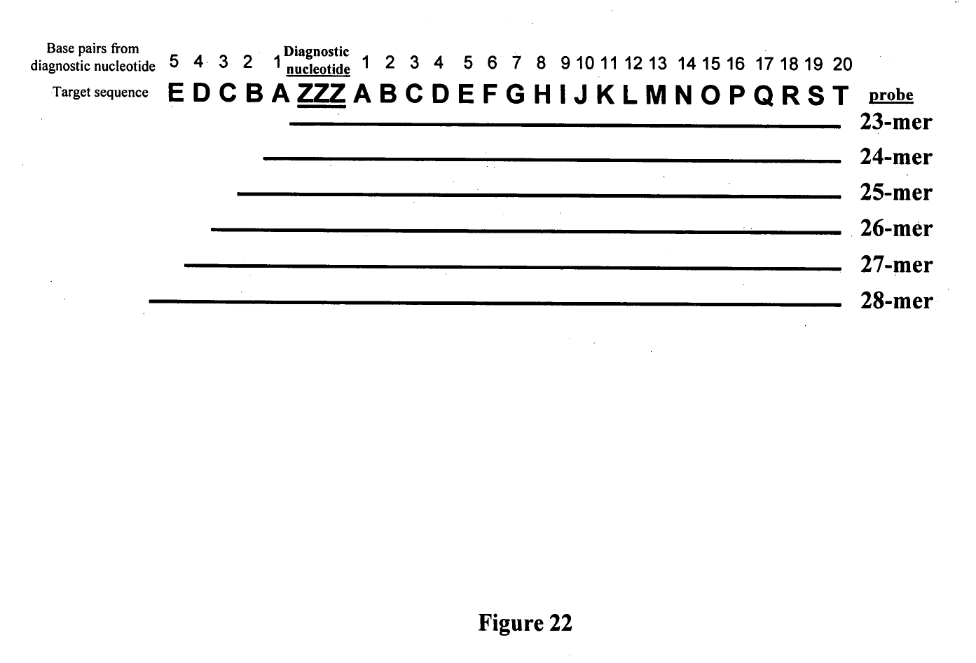

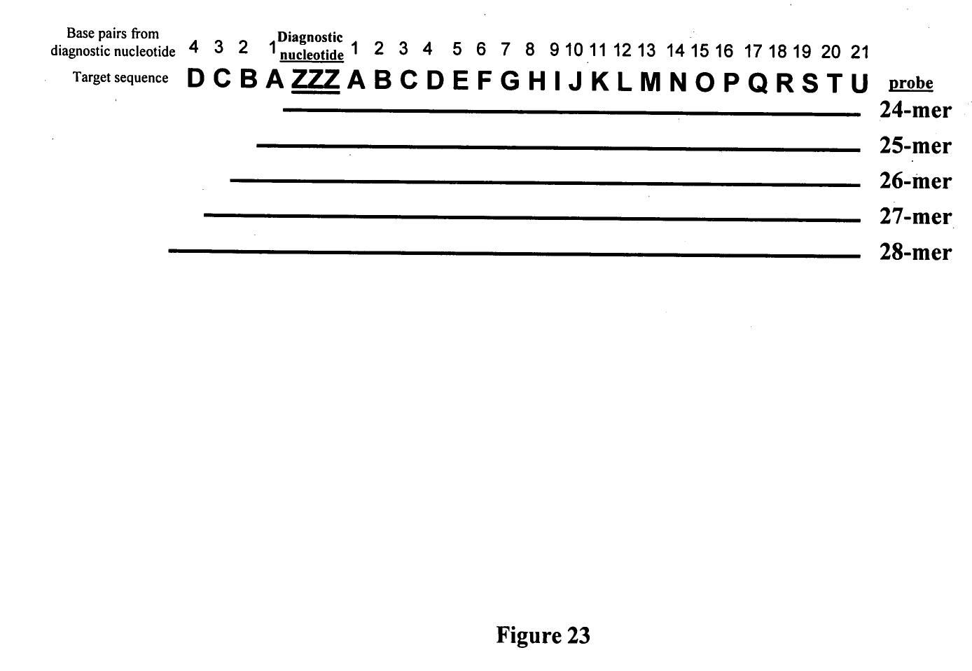

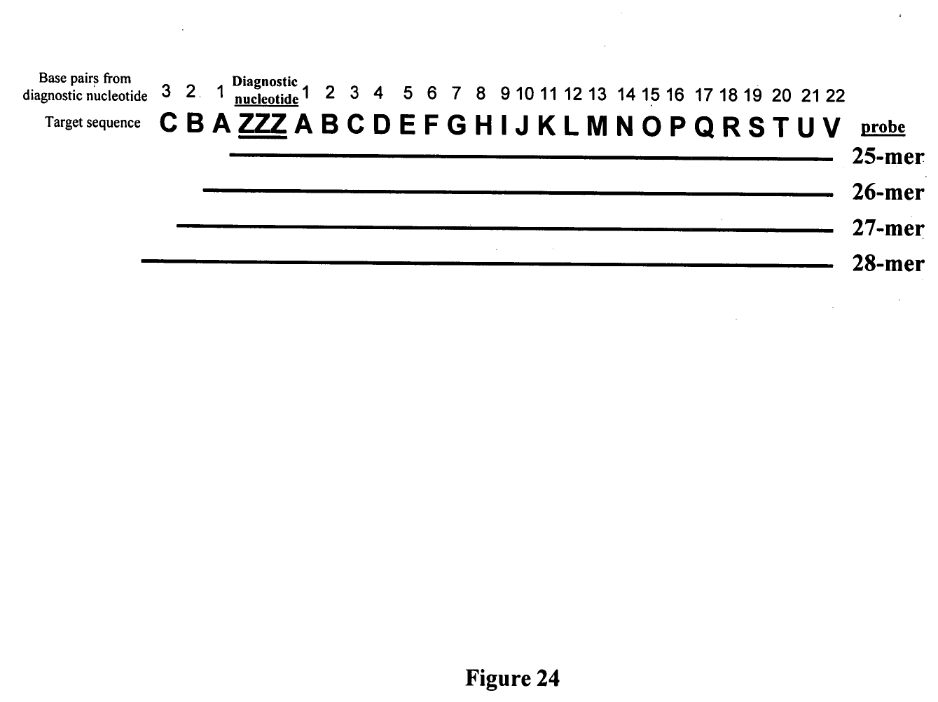

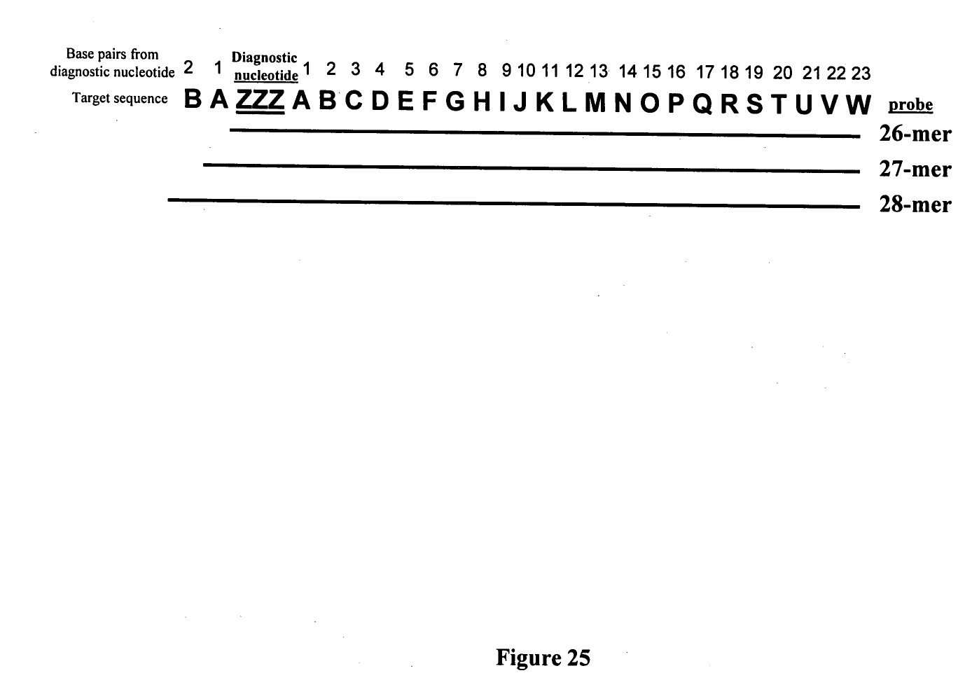

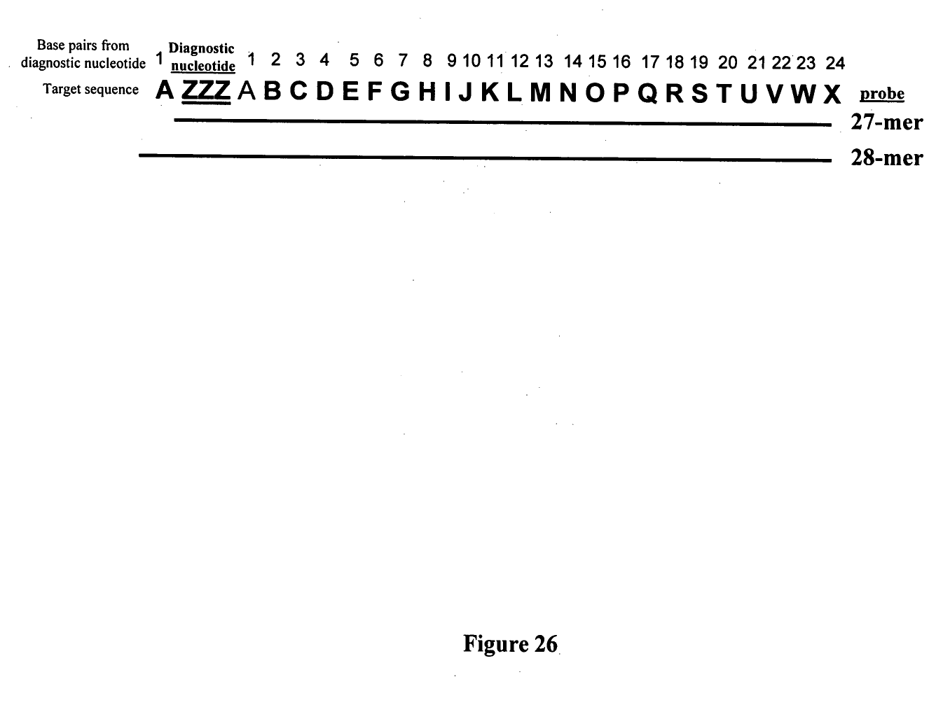

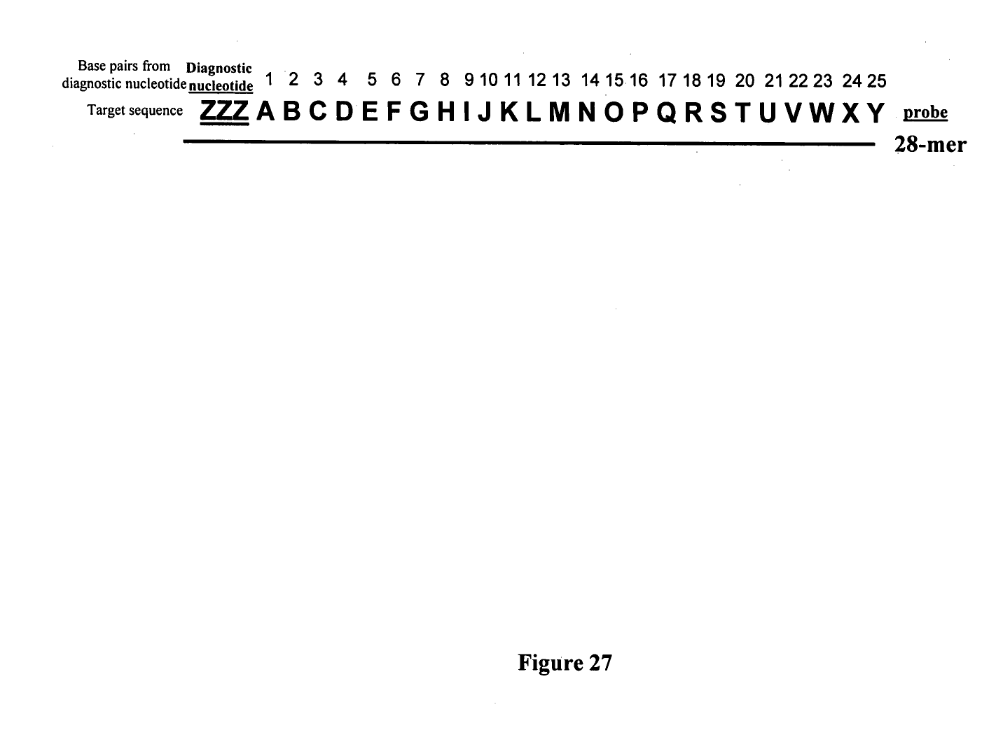

FIGS. 2-27 are schematic illustration of target nucleic acid sequence which contains a diagnostic nucleotide codon/nucleic acid(s) ZZZ. Alphabetic letters are used independently to symbolize nucleotide bases. These Figures are read in conjunction with Tables 1-13, which illustrate nucleic acid sequences encompassing codons or nucleic acids that are diagnostic for a long term response to 3TC therapy. Each nucleic acid sequence in Tables 1-13 is a 28mer. However, according to the invention any nucleic acid sequence from 14-28 or 30 bases in length can be used in the diagnostic process, material and kit. Therefore, FIGS. 2-27 are intended to constitute specific disclosures of each 14 through 28mer of each nucleic acid sequence in Tables 1-13. Complementary probes to the target sequence are also intended to be likewise specifically depicted by these Figures.

TABLE 1
Nonlimiting examples of nucleic acid sequences encompassing codon for amino acid 91
of the HBV polymerase. For purposes of probe construction, probes to detect the diagnostic nucleotide
can be constructed containing additional or fewer nucleotides flanking the diagnostic nucleotide than the
28 base pair size represented below.

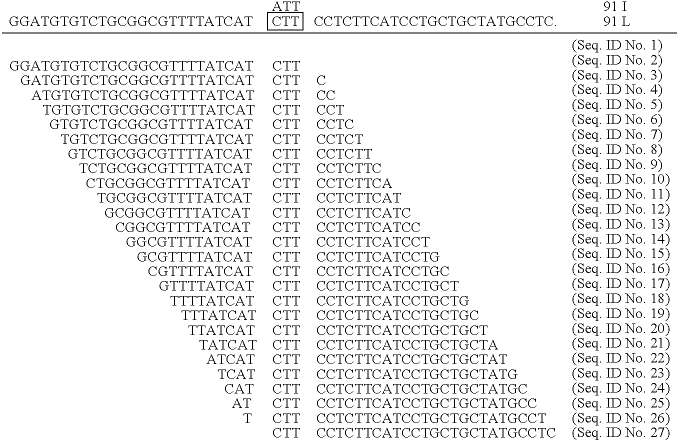

Note: The following additional codons an encode leucine at amino acid 91: TTA, TTG, CTC, CTA, or CTG. These nucleotides can be substituted for the CTT sequence that is listed above in Sequence ID Nos. 1-27. In addition, the corresponding amino acid sequences of the HBV viral markers diagnostic for long term response of HBV carriers to 3TC therapy can be determined from the illustrative HBV nucleotide sequences above.

TABLE 2
Nonlimiting examples of nucleic acid sequences encompassing codon for amino acid 256 of the HBV polymerase.
For purposes of probe construction, probes to detect the diagnostic nucleotide can be constructed containing
additional or fewer nucleotides flanking the diagnostic nucleotide than the 28 base pair size represented below.

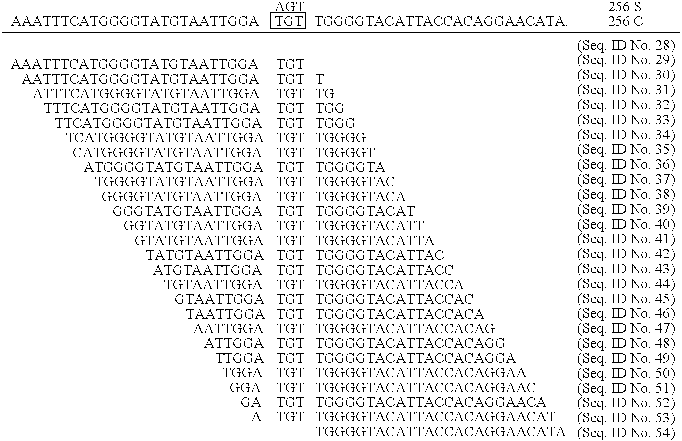

Note: The codon TGC can also encode cysteine at amino acid 256; this nucleotide triplet can be substituted for the TGT sequence that is listed above in Sequence ID Nos. 28-54. In addition, the corresponding amino acid sequences of the HBV viral markers diagnostic for long term response of HBV carriers to 3TC therapy can be determined from the illustrative HBV nucleotide sequences.

TABLE 3
Nonlimiting examples of nucleic acid sequences encompassing codon for amino acid 213 of the HBV polymerase.
For purposes of probe construction, probes to detect the diagnostic nucleotide can be constructed containing
additional or fewer nucleotides flanking the diagnostic nucleotide than the 28 base pair size represented below.

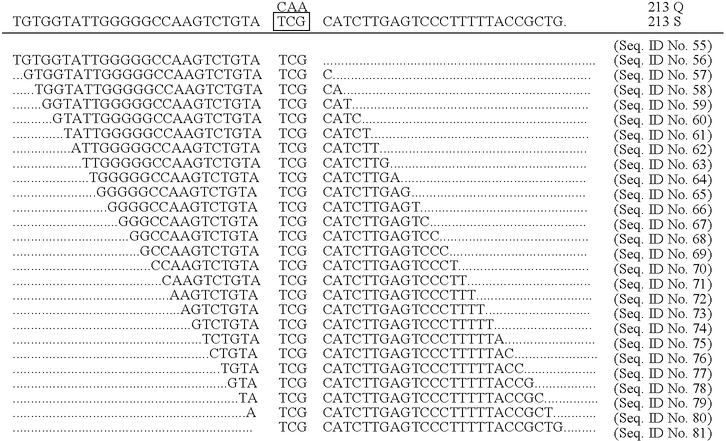

Note: The codons TCA, TCC, TCT, AGT, AGC, and TCG can also encode serine at amino acid 213, these nucleotide triplets can be substituted for the TCG sequence that is listed above in Sequence ID Nos. 56-81. In addition, the corresponding amino acid sequences of the HBV viral markers diagnostic for long term response of HBV carriers to 3TC therapy can be determined from the illustrative HBV nucleotide sequences

TABLE 4
Nonlimiting examples of nucleic acid sequences encompassing a single point mutation at nucleotide 1739 of the DNA
precore/core promoter or open reading frame (ORF) region of HBV. For purposes of probe construction,
probes to detect the diagnostic nucleotide can be constructed containing additional or fewer
nucleotides flanking the diagnostic nucleotide than the 28 base pair size represented below.

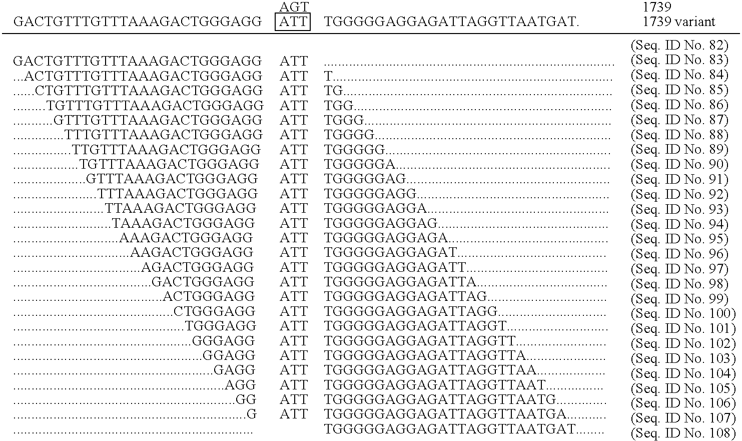

In addition, the corresponding amino acid sequences of the HBV viral markers diagnostic for long term response of HBV carriers to 3TC therapy can be determined from the illustrative HBV nucleotide sequences

TABLE 5
Nonlimiting examples of nucleic acid sequences encompassing a single point mutation at nucleotide 1752
of the DNA precore/core promoter or open reading frame (ORF) region of HBV. For purposes of probe construction,
probes to detect the diagnostic nucleotide can be constructed containing additional or fewer nucleotides flanking
the diagnostic nucleotide than the 28 base pair size represented below.

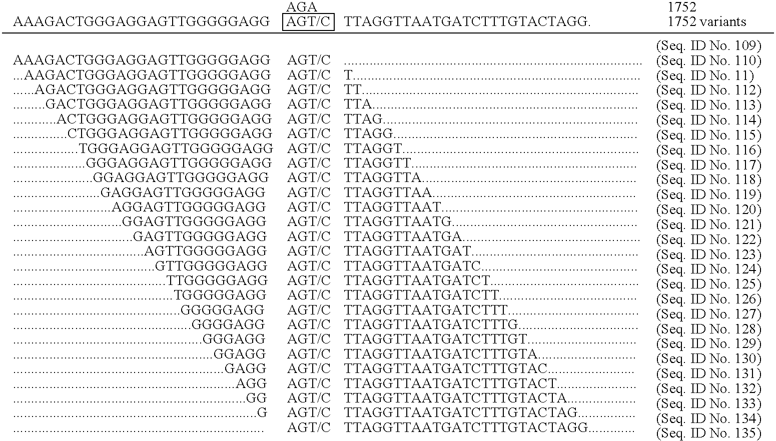

In addition, the corresponding amino acid sequences of the HBV viral markers diagnostic for long term response of HBV carriers to 3TC therapy can be determined from the illustrative HBV nucleotide sequences

TABLE 6
Nonlimiting examples of nucleic acid sequences encompassing a single point mutation
at nucleotide 1909 of the DNA precore/core promoter or open reading frame (ORF) region of
HBV. For purposes of probe construction, probes to detect the diagnostic nucleotide can be
constructed containing additional or fewer nucleotides flanking the diagnostic nucleotide than the
28 base pair size represented below.

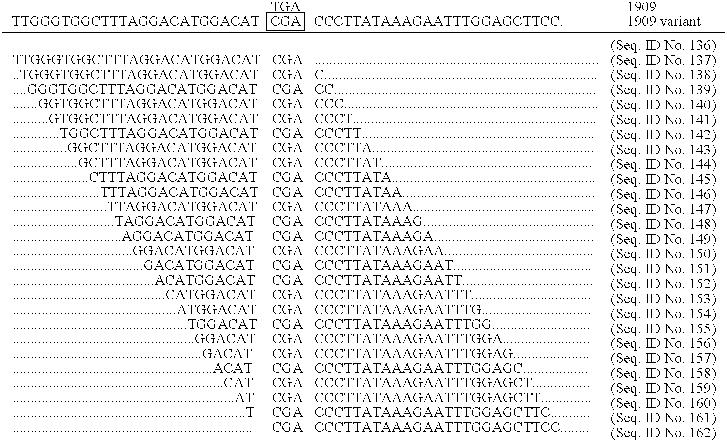

In addition, the corresponding amino acid sequences of the HBV viral markers diagnostic for long term response of HBV carriers to 3TC therapy can be determined from the illustrative HBV nucleotide sequences

TABLE 7
Nonlimiting examples of nucleic acid sequences encompassing a single point mutation
at nucleotide 1960 of the DNA precore/core promoter or open reading frame (ORF) region of
HBV. For purposes of probe construction, probes to detect the diagnostic nucleotide can be
constructed containing additional or fewer nucleotides flanking the diagnostic nucleotide than the
28 base pair size represented below.

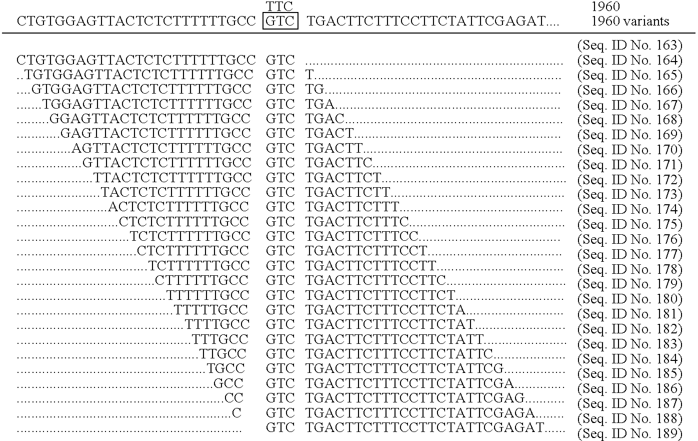

In addition, the corresponding amino acid sequences of the HBV viral markers diagnostic for long term response of HBV carriers to 3TC therapy can be determined from the illustrative HBV nucleotide sequences

TABLE 8
Nonlimiting examples of nucleic acid sequences encompassing a single point mutation
at nucleotide 1961 of the DNA precore/core promoter or open reading frame (ORF) region of
HBV. For purposes of probe construction, probes to detect the diagnostic nucleotide can be
constructed containing additional or fewer nucleotides flanking the diagnostic nucleotide than the
28 base pair size represented below.

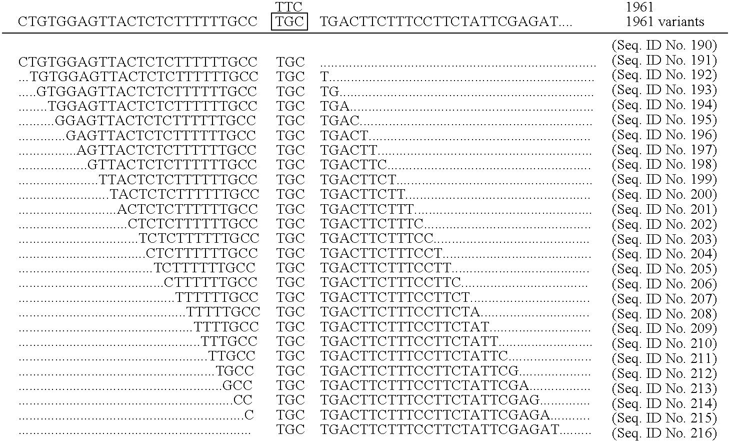

In addition, the corresponding amino acid sequences of the HBV viral markers diagnostic for long term response of HBV carriers to 3TC therapy can be determined from the illustrative HBV nucleotide sequences.

TABLE 9
Nonlimiting examples of nucleic acid sequences encompassing a single point mutation
at nucleotide 1961 of the DNA precore/core promoter or open reading frame (ORF) region of
HBV. For purposes of probe construction, probes to detect the diagnostic nucleotide can be
constructed containing additional or fewer nucleotides flanking the diagnostic nucleotide than the
28 base pair size represented below.

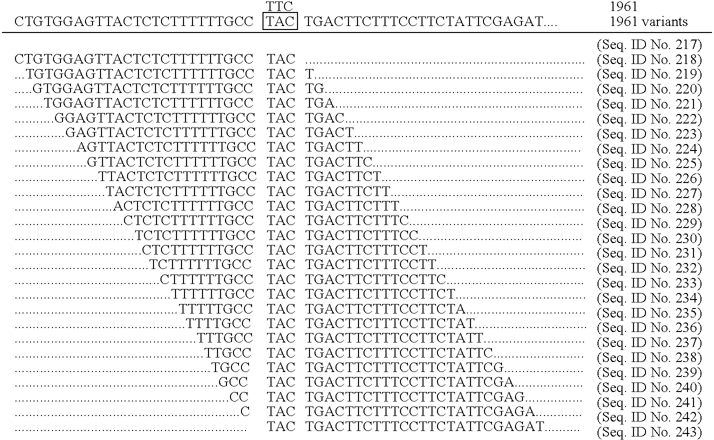

In addition, the corresponding amino acid sequences of the HBV viral markers diagnostic for long term response of HBV carriers to 3TC therapy can be determined from the illustrative HBV nucleotide sequences

TABLE 10
Nonlimiting examples of nucleic acid sequences encompassing double point mutations
at nucleotides 1738 and 1739 of the DNA precore/core promoter or open reading frame (ORF)
region of HBV. For purposes of probe construction, probes to detect the diagnostic nucleotide
can be constructed containing additional or fewer nucleotides flanking the diagnostic nucleotide
than the 28 base pair size represented below.

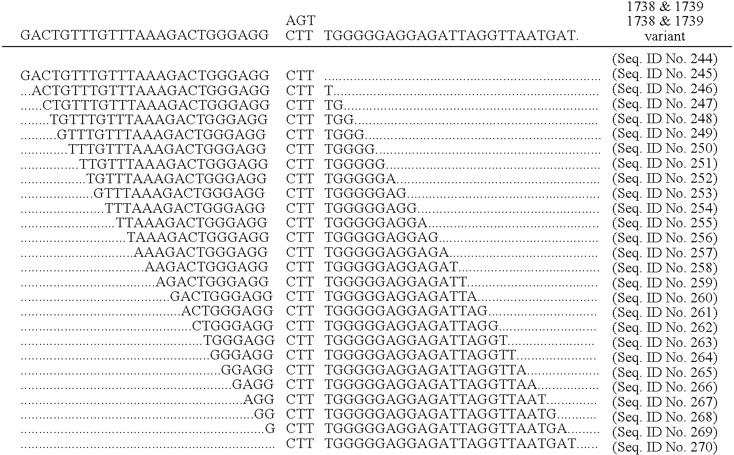

In addition, the corresponding amino acid sequences of the HBV viral markers diagnostic for long term response of HBV carriers to 3TC therapy can be determined from the illustrative HBV nucleotide sequences

TABLE 11
Nonlimiting examples of nucleic acid sequences encompassing double point mutations
at nucleotides 1750 and 1752 of the DNA precore/core promoter or open reading frame (ORF)
region of HBV. For purposes of probe construction, probes to detect the diagnostic nucleotide
can be constructed containing additional or fewer nucleotides flanking the diagnostic nucleotide
than the 28 base pair size represented below.

| | | | 1750 & 1752 |
|---|---|---|---|
| | | TTAGGTTAATGATCTTTGTACTAGG. | 1750 & 1752 |
| AAAGACTGGGAGGAGTTGGGGGAGG | 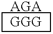 | (Seq. ID No.271) | variant |
| AAAGACTGGGAGGAGTTGGGGGAGG | GGG | ............................... | (Seq. ID No.272) |
| ..AAGACTGGGAGGAGTTGGGGAGG | GGG | T.............................. | (Seq. ID No.273) |
| ..AGACTGGGAGGAGTTGGGGGAGG | GGG | TT............................. | (Seq. ID No.274) |
| ...GACTGGGAGGAGTTGGGGGAGG | GGG | TTA............................ | (Seq. ID No.275) |
| ....ACTGGGAGGAGTTGGGGGAGG | GGG | TTAG........................... | (Seq. ID No.276) |
| .....CTGGGAGGAGTTGGGGGAGG | GGG | TTAGG.......................... | (Seq. ID No.277) |
| ......TGGGAGGAGTTGGGGGAGG | GGG | TTAGGT......................... | (Seq. ID No.278) |
| .......GGGAGGAGTTGGGGGAGG | GGG | TTAGGTT........................ | (Seq. ID No.279) |
| ........GGAGGAGTTGGGGGAGG | GGG | TTAGGTTA....................... | (Seq. ID No.280) |
| .........GAGGAGTTGGGGGAGG | GGG | TTAGGTTAA...................... | (Seq. ID No.281) |
| ..........AGGAGTTGGGGGAGG | GGG | TTAGGTTAAT..................... | (Seq. ID No.282) |
| ...........GGAGTTGGGGGAGG | GGG | TTAGGTTAATG.................... | (Seq. ID No.283) |
| ............GAGTTGGGGGAGG | GGG | TTAGGTTAATGA................... | (Seq. ID No.284) |
| .............AGTTGGGGGAGG | GGG | TTAGGTTAATGAT.................. | (Seq. ID No.285) |
| ..............GTTGGGGGAGG | GGG | TTAGGTTAATGATC................. | (Seq. ID No.286) |
| ...............TTGGGGGAGG | GGG | TTAGGTTAATGATCT................ | (Seq. ID No.287) |
| ................TGGGGGAGG | GGG | TTAGGTTAATGATCTT............... | (Seq. ID No.288) |
| .................GGGGGAGG | GGG | TTAGGTTAATGATCTTT.............. | (Seq. ID No.289) |
| ..................GGGGAGG | GGG | TTAGGTTAATGATCTTTG............. | (Seq. ID No.290) |
| ...................GGGAGG | GGG | TTAGGTTAATGATCTTTGT............ | (Seq. ID No.291) |
| ....................GGAGG | GGG | TTAGGTTAATGATCTTTGTA........... | (Seq. ID No.292) |
| .....................GAGG | GGG | TTAGGTTAATGATCTTTGTAC.......... | (Seq. ID No.293) |
| ......................AGG | GGG | TTAGGTTAATGATCTTTGTACT......... | (Seq. ID No.294) |
| .......................GG | GGG | TTAGGTTAATGATCTTTGTACTA........ | (Seq. ID No.295) |
| ........................G | GGG | TTAGGTTAATGATCTTTGTACTAG....... | (Seq. ID No.296) |
| ......................... | GGG | TTAGGTTAATGATCTTTGTACTAGG...... | (Seq. ID No.297) |

In addition, the corresponding amino acid sequences of the HBV viral markers diagnostic for long term response of HBV carriers to 3TC therapy can be determined from the illustrative HBV nucleotide sequences

TABLE 12
Nonlimiting examples of nucleic acid sequences encompassing double point mutations
at nucleotides 1909 and 1911 of the DNA precore/core promoter or open reading frame (ORF)
region of HBV. For purposes of probe construction, probes to detect the diagnostic nucleotide
can be constructed containing additional or fewer nucleotides flanking the diagnostic nucleotide
than the 28 base pair size represented below.

| | | | 1909 & 1911 |
|---|---|---|---|
| | | CCCTTATAAAGAATTTGGAGCTTCC. | 1909 & 1911 |
| TTGGGTGGCTTTAGGACATGGACAT | 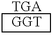 | (Seq. ID No.298) | variant |
| TTGGGTGGCTTTAGGACATGGACAT | GGT | ............................... | (Seq. ID No.299) |
| .TGGGTGGCTTTAGGACATGGACAT | GGT | C.............................. | (Seq. ID No.300) |
| ..GGGTGGCTTTAGGACATGGACAT | GGT | CC............................. | (Seq. ID No.301) |
| ...GGTGGCTTTAGGACATGGACAT | GGT | CCC............................ | (Seq. ID No.302) |
| ....GTGGCTTTAGGACATGGACAT | GGT | CCCT........................... | (Seq. ID No.303) |
| .....TGGCTTTAGGACATGGACAT | GGT | CCCTT.......................... | (Seq. ID No.304) |
| ......GGCTTTAGGACATGGACAT | GGT | CCCTTA......................... | (Seq. ID No.305) |
| .......GCTTTAGGACATGGACAT | GGT | CCCTTAT........................ | (Seq. ID No.306) |
| ........CTTTAGGACATGGACAT | GGT | CCCTTATA....................... | (Seq. ID No.307) |
| .........TTTAGGACATGGACAT | GGT | CCCTTATAA...................... | (Seq. ID No.308) |
| ..........TTAGGACATGGACAT | GGT | CCCTTATAAA..................... | (Seq. ID No.309) |
| ...........TAGGACATGGACAT | GGT | CCCTTATAAAG.................... | (Seq. ID No.310) |
| ............AGGACATGGACAT | GGT | CCCTTATAAAGA................... | (Seq. ID No.311) |
| .............GGACATGGACAT | GGT | CCCTTATAAAGAA.................. | (Seq. ID No.312) |
| ..............GACATGGACAT | GGT | CCCTTATAAAGAAT................. | (Seq. ID No.313) |
| ...............ACATGGACAT | GGT | CCCTTATAAAGAATT................ | (Seq. ID No.314) |
| ................CATGGACAT | GGT | CCCTTATAAAGAATTT............... | (Seq. ID No.315) |
| .................ATGGACAT | GGT | CCCTTATAAAGAATTTG.............. | (Seq. ID No.316) |
| ..................TGGACAT | GGT | CCCTTATAAAGAATTTGG............. | (Seq. ID No.317) |
| ...................GGACAT | GGT | CCCTTATAAAGAATTTGGA............ | (Seq. ID No.318) |
| ....................GACAT | GGT | CCCTTATAAAGAATTTGGAG........... | (Seq. ID No.319) |
| .....................ACAT | GGT | CCCTTATAAAGAATTTGGAGC.......... | (Seq. ID No.320) |
| ......................CAT | GGT | CCCTTATAAAGAATTTGGAGCT......... | (Seq. ID No.321) |
| .......................AT | GGT | CCCTTATAAAGAATTTGGAGCTT........ | (Seq. ID No.322) |
| ........................T | GGT | CCCTTATAAAGAATTTGGAGCTTC....... | (Seq. ID No.323) |
| ......................... | GGT | CCCTTATAAAGAATTTGGAGCTTCC...... | (Seq. ID No.324) |

In addition, the corresponding amino acid sequences of the HBV viral markers diagnostic for long term response of HBV carriers to 3TC therapy can be determined from the illustrative HBV nucleotide sequences

TABLE 13
Nonlimiting examples of nucleic acid sequences encompassing double point mutations
at nucleotides 1961 and 1962 of the DNA precore/core promoter or open reading frame (ORF)
region of HBV. For purposes of probe construction, probes to detect the diagnostic nucleotide
can be constructed containing additional or fewer nucleotides flanking the diagnostic nucleotide
than the 28 base pair size represented below.

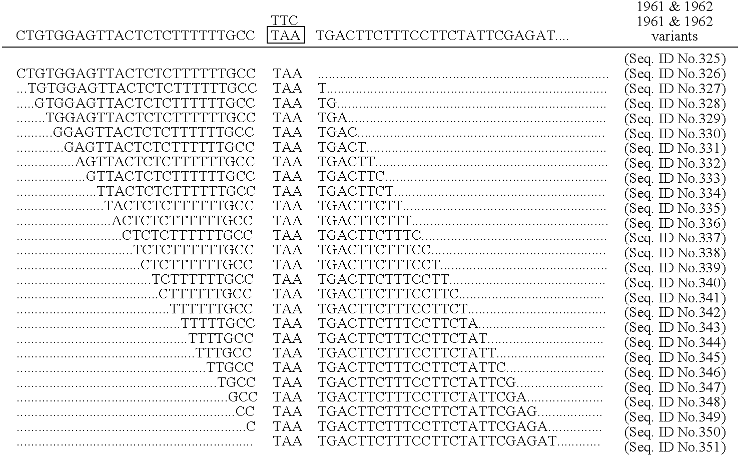

In addition, the corresponding amino acid sequences of the HBV viral markers diagnostic for long term response of HBV carriers to 3TC therapy can be determined from the illustrative HBV nucleotide sequences

In addition to identifying viral markers for the purposes of identifying HBV strains that are correlated with lamivudine failure, the present invention can be utilized to also identify HBV strains that respond to lamivudine therapy. In this respect, the absence of viral markers correlated with lamivudine therapy can be used to prescribe a course of treatment that includes lamivudine as a modality for those individuals that carrier HBV lacking viral markers correlated with lamivudine therapy failure.

In another embodiment, the invention provides an oligonucleotide primer for amplifying an HBV nucleic acid sequence. In one embodiment, the oligonucleotide is at least 14 nucleotides in length and hybridizes under sequence-specific, stringent hybridization conditions to a nucleotide sequence that contains the marker correlated with therapy failure.

Oligonucleotide sequences used as the hybridizing region of a primer can also be used as the hybridizing region of a probe. Suitability of a primer sequence for use as a probe depends on the hybridization characteristics of the primer. Similarly, an oligonucleotide used as a probe can be used as a primer.

It will be apparent to those of skill in the art that, provided with those embodiments, specific primers and probes can be prepared by, for example, the addition of nucleotides to either the 5′ or 3′ ends, which nucleotides are complementary to the target sequence or are not complimentary to the target sequence. So long as primer compositions serve as a point of initiation for extension on the target sequences, and so long as the primers and probes comprise at least 14 consecutive nucleotides contained within those exemplified embodiments, such compositions are within the scope of the invention.

The probe(s) herein can be selected by the following criteria, which are factors to be considered, but are not exclusive or determinative. The probes are selected from the region of the HBV genome that contains the nucleic acid viral marker. The probe lacks homology with any sequences of vital genomes that would be expected to compromise the test. The probe lacks secondary structure formation in the amplified nucleic acid which can interfere with extension by the amplification enzyme such as E. coli DNA polymerase, preferably that portion of the DNA polymerase referred to as the Klenow fragment. This can be accomplished by employing up to about 15% by weight, preferably 5-10% by weight, dimethyl sulfoxide (DMSO) in the amplification medium and/or increasing the amplification temperatures to 30°-40° C.

Preferably, the probe should contain approximately 50% guanine and cytosine nucleotides, as measured by the formula adenine (A)+thymine (T)+cytosine (C)+guanine (G)/cytosine (C)+guanine (G). Preferably, the probe does not contain multiple consecutive adenine and thymine residues at the 3′ end of the primer which can result in less stable hybrids.

The probes of the invention can be about 10 to 30 nucleotides long, preferably at least 10, 11, 12, 13, 14, 15, 20, 25, or 28 nucleotides in length, including specifically 14, 15, 16, 17, 18, 19, 20, 21, 22, 23, 24, 25, 26, 27, 28, 29 or 30 nucleotides. The nucleotides as used in the present invention can be ribonucleotides, deoxyribonucleotides and modified nucleotides such as inosine or nucleotides containing modified groups which do not essentially alter their hybridization characteristics. Probe sequences are represented throughout the specification as single stranded DNA oligonucleotides from the 5′ to the 3′ end. Any of the probes can be used as such, or in their complementary form, or in their RNA form (wherein T is replaced by U).

The probes according to the invention can be prepared by cloning of recombinant plasmids containing inserts including the corresponding nucleotide sequences, optionally by cleaving the latter out from the cloned plasmids upon using the adequate nucleases and recovering them, e.g. by fractionation according to molecular weight. The probes according to the present invention can also be synthesized chemically, for instance by the conventional phosphotriester or phosphodiester methods or automated embodiments thereof. In one such automated embodiment diethylphosphoramidites are used as starting materials and can be synthesized as described by Beaucage et al., Tetrahedron Letters 22:1859-1862 (1981). One method of synthesizing oligonucleotides on a modified solid support is described in U.S. Pat. No. 4,458,066. It is also possible to use a primer which has been isolated from a biological source (such as a restriction endonuclease digest).

The oligonucleotides used as primers or probes can also comprise nucleotide analogues such as phosphorothiates (Matsukura S., Naibunpi Gakkai Zasshi. 43(6):527-32 (1967)), alkylphosphorothiates (Miller P. et al., Biochemistry 18(23):5134-43 (1979), peptide nucleic acids (Nielsen P. et al., Science 254(5037):1497-500 (1991); Nielsen P. et al., Nucleic-Acids-Res. 21(2):197-200 (1993)), morpholino nucleic acids, locked nucleic acids, pseudocyclic oligonucleobases, 2′-O,4′-C-ethylene bridged nucleic acids or can contain intercalating agents (Asseline J. et al., Proc. Natl. Acad. Sci. USA 81(11):3297-301 (1984)).

For designing probes with desired characteristics, the following useful guidelines known to the person skilled in the art can be applied. Because the extent and specificity of hybridization reactions are affected by a number of factors, manipulation of one or more of those factors will determine the exact sensitivity and specificity of a particular probe, whether perfectly complementary to its target or not. The importance and effect of various assay conditions, explained further herein, are known to those skilled in the art.

The stability of the probe to target nucleic acid hybrid should be chosen to be compatible with the assay conditions. This can be accomplished by avoiding long AT-rich sequences, by terminating the hybrids with GC base pairs, and/or by designing the probe with an appropriate Tm. The beginning and end points of the probe should be chosen so that the length and % GC result in a Tm about 2-10° C. higher than the temperature at which the final assay will be performed. The base composition of the probe is significant because G-C base pairs exhibit greater thermal stability compared to A-T base pairs due to additional hydrogen bonding. Thus, hybridization involving complementary nucleic acids of higher G-C content will be stable at higher temperatures. Conditions such as ionic strength and incubation temperature under which probe will be used should also be taken into account when designing a probe. It is known that hybridization will increase as the ionic strength of the reaction mixture increases, and that the thermal stability of the hybrids will increase with increasing ionic strength. Chemical reagents, such as formamide, urea, DIVISO and alcohols, which disrupt hydrogen bonds, will increase the stringency of hybridization. Destabilization of the hydrogen bonds by such reagents can greatly reduce the Tm. In general, optimal hybridization for synthetic oligonucleotide probes of about 10-50 bases in length occurs approximately 5° C. below the melting temperature for a given duplex. Incubation at temperatures below the optimum can allow mismatched base sequences to hybridize and can therefore result in reduced specificity. It is desirable to have probes which hybridize only under conditions of high stringency. Under high stringency conditions only highly complementary nucleic acid hybrids will form; hybrids without a sufficient degree of complementarity will not form. Accordingly, the stringency of the assay conditions determines the amount of complementarity needed between two nucleic acid strands forming a hybrid. The degree of stringency is chosen such as to maximize the difference in stability between the hybrid formed with the target and the non-target nucleic acid. In the present case, single base pair changes need to be detected, which requires conditions of very high stringency.

The length of the target nucleic acid sequence and, accordingly, the length of the probe sequence can also be important. In some cases, there can be several sequences from a particular region, varying in location and length, which will yield probes with the desired hybridization characteristics. In other cases, one sequence can be significantly better than another which differs merely by a single base.

While it is possible for nucleic acids that are not perfectly complementary to hybridize, the longest stretch of perfectly complementary base sequence will normally primarily determine hybrid stability. While oligonucleotide probes of different lengths and base composition can be used, preferred oligonucleotide probes of this invention are between about 14 and 30 bases in length and have a sufficient stretch in the sequence which is perfectly complementary to the target nucleic acid sequence.

Regions in the target DNA or RNA which are known to form strong internal structures inhibitory to hybridization are less preferred. Likewise, probes with extensive self-complementarity should be avoided. As explained above, hybridization is the association of two single strands of complementary nucleic acids to form a hydrogen bonded double strand. It is implicit that if one of the two strands is wholly or partially involved in a hybrid, it will be less able to participate in formation of a new hybrid. There can be intramolecular and intermolecular hybrids formed within the molecules of one type of probe if there is sufficient self complementarity. Such structures can be avoided through careful probe design. By designing a probe so that a substantial portion of the sequence of interest is single stranded, the rate and extent of hybridization can be greatly increased. Computer programs are available to search for this type of interaction. However, in certain instances, it may not be possible to avoid this type of interaction.

Specific primers and sequence specific oligonucleotide probes can be used in a polymerase chain reaction that enables amplification and detection of the viral genomic sequences.

One aspect of the invention relates to specific oligonucleotide primers. The invention provides compositions comprising an oligonucleotide primer for amplifying an HBV nucleic acid wherein said primer is suitable for amplifying a nucleic acid subsequence from an HBV nucleic acid viral marker.

2. Detection of Hybridization of the Probe and Target Sequence

Suitable assay formats for detecting hybrids formed between probes and target nucleic acid sequences in a sample are known in the art (Sambrook et al., EMBO J. 4(1):91-103 (1985); Nature Genetics 21 (supplement): 1-60 (1999)). Detection of hybridization can be accomplished whether or not the nucleic acid has been amplified.

Oligonucleotides can be labeled by incorporating a label detectable by spectroscopic, photochemical, biochemical, immunochemical, or chemical means. Useful labels include ³²P, fluorescent dyes, electron-dense reagents, enzymes (as commonly used in ELISAS), biotin, or, haptens and proteins for which antisera or monoclonal antibodies are available.

The nucleic acid can be detected by analyzing it by Southern blots with or without using radioactive probes. In one embodiment, a small sample of DNA from, e.g., peripheral blood suspected of containing HBV, is analyzed via a Southern blotting technique using oligonucleotide probes to detect the specific nucleic acid viral marker. In another embodiment, a small sample of DNA from, e.g., peripheral blood suspected of containing HBV, is first amplified and then analyzed via a Southern blotting technique using oligonucleotide probes to detect the specific nucleic acid viral marker.

Another method of detection involves detection using a labeled probe capable of hybridizing with the amplified nucleic acid sequence and determining if the probe has hybridized. Such probe necessarily contains a nucleic acid sequence of a viral marker of HBV.

Another method involves the oligomer restriction technique (such as described in U.S. Pat. No. 4,683,194). In this procedure, the amplified nucleic acid is denatured and hybridized in solution to a labeled oligonucleotide probe which hybridizes specifically to the target sequence (spans the particular conserved region contained by the primers) and spans at least one restriction site of interest. The duplex formed between the target and probe will reconstitute the restrictions site, and when cleaved with restriction enzyme, such as, e.g., BglI, PvuII, or HifI, releases a labeled probe fragment which can be resolved from the full-length probe by gel electrophoresis. The resulting gel is then autoradiographed. Analysis of the amplified product by this method can be rapid, i.e., results can be obtained in a few hours.

Another method which can be used to analyze the amplified product is the dot blot method. In a dot-blot method, amplified target DNA is immobilized on a solid support, such as a nylon membrane. The membrane-target complex is incubated with labeled probe under suitable hybridization conditions, unhybridized probe is removed by washing under suitably stringent conditions, and the membrane is monitored for the presence of bound probe.

An alternate format is a “reverse” dot-blot format, in which the amplified target DNA is labeled and the probes are immobilized on a solid support, such as a nylon membrane (see Saiki et al., Proc. Natl. Acad. Sci. USA 86:6230 (1989), and PCT Patent Publication No. 89/11548). The target DNA is typically labeled during amplification by the incorporation of labeled primers. One or both of the primers can be labeled. The membrane-probe complex is incubated with the labeled amplified target DNA under suitable hybridization conditions, unhybridized target DNA is removed by washing under suitably stringent conditions, and the filter is then monitored for the presence of bound target DNA.

Alternatively, the reverse dot-blot assay can be carried out using a solid support having a plurality of probe hybridization sites or wells. For example, a microwell plate is particularly useful in large scale clinical applications of the present methods. Probes can be immobilized to a microwell plate either by passive binding or through a protein intermediate, such as bovine serum albumin (BSA), which adheres to microwell plates. Reverse dot-blot methods carried out in a microwell plate are described in U.S. Pat. No. 5,232,829, and Loeffelholz et al, J. Clin. Microbiol. 30(11):2847-2851 (1992). In another embodiment of the invention, a reverse dot-blot assay is carried out using microwell plates, and the primers are labeled with biotin, as described in Levenson and Chang, 1989, in PCR Protocols: A Guide to Methods and Applications, (Innis et al., eds., Academic Press. San Diego) pages 99-112, incorporated herein by reference. The probes are conjugated with BSA (see Tung et al., Bioconjugate Chem. 2:464-465 (1991), incorporated herein by reference) and immobilized on a microwell plate. Following amplification using the labeled primers and hybridization with the immobilized probes, the amplified nucleic acid is detected by first binding the biotin to avidin-horseradish peroxidase (A-HRP) or streptavidin-horseradish peroxidase (SAHRP), which is then detected by carrying out a reaction in which the HRP catalyzes a color change of a chromogen

In an alternative method of immobilizing hybridization duplexes for detection, BSA-conjugated probes are bound to magnetic microparticles. The bound probes are hybridized in solution to labeled amplification product. Following hybridization, probe-target duplexes are removed from the solution magnetically, and the magnetically immobilized hybridization duplexes are then detected as in the methods described above.

Another method of detection is referred to as a 5′-nuclease assay in which the labeled detection probes are added during the PCR amplification process. The probes are modified so as to prevent the probes from acting as primers for DNA synthesis. Any probe which hybridizes to target DNA during each synthesis step, i.e., during primer extension, is degraded by the 5′ to 3′ exonuclease activity of the DNA polymerase, e.g., Taq DNA polymerase. The degradation product from the probe is then detected. Thus, the presence of probe breakdown product indicates both that hybridization between probe and target DNA occurred and that the amplification reaction occurred. See also for example, U.S. Pat. No. 5,210,015.

Another method of detection is through northern blot analysis. For example, in one embodiment, HBV RNA is isolated from cells, tissue or serum infected with HBV (see, for example, U.S. Pat. No. 5,128,247). For the Northern blot, the RNA is denatured in an appropriate buffer (such as glyoxal/dimethyl, sulfoxide/sodium phosphate buffer), subjected to agarose gel electrophoresis, and transferred onto a nitrocellulose or nylon filter. The applicable viral marker probe is labeled according to any appropriate method (such as the ³²P-multiprimed DNA labeling system (Amersham)) is used as probe. After hybridization, the filter is washed and exposed to x-ray film to determine the presence of radiolabeled probe. See also, for example, Harada et al., Cell 63:303-312 (1990).

S1 mapping can also be performed as described in Fujita et al., Cell 49:357-367 (1987). To prepare probe DNA for use in S1 mapping, the sense strand representing Seq IDs 1-351 is used as a template to synthesize labeled antisense DNA. The antisense DNA can then be digested using an appropriate restriction endonuclease to generate further DNA probes of a desired length. Such antisense probes are useful for visualizing protected bands corresponding to viral marker mRNA. Northern blot analysis can be performed as described above.

Alternatively, the presence of viral markers can be assayed using the RT-PCR method described in Makino et al., Technique 2:295-301 (1990). Briefly, this method involves adding total RNA isolated from a biological sample in a reaction mixture containing a RT primer and appropriate buffer. After incubating for primer annealing, the mixture can be supplemented with a RT buffer, dNTPs, DTT, RNase inhibitor and reverse transcriptase. After incubation to achieve reverse transcription of the RNA, the RT products are then subject to PCR using specific primers, such as those generated from Seq IDs 1-351 to amplify the viral markers. After a suitable number of rounds to achieve amplification, the PCR reaction mixture is electrophoresed on a polyacrylamide gel and analyzed for the presence of bands representative of the presence of the viral marker. Alternatively, the amplified products can be sequenced as described above. RT and PCR reaction ingredients and conditions, reagent and gel concentrations, and methods are well known in the art. Variations on the RT-PCR method will be apparent to the skilled artisan.

The assay formats described above typically utilize labeled oligonucleotides to facilitate detection of the hybrid duplexes. Oligonucleotides can be labeled by incorporating a label detectable by spectroscopic, photochemical, biochemical, immunochemical, or chemical means. Useful labels include ³²P, fluorescent dyes, electron-dense reagents, enzymes (as commonly used in ELISAS), biotin, or haptens and proteins for which antisera or monoclonal antibodies are available.

An alternative method for detecting the amplification of HBV nucleic acid is by monitoring the increase in the total amount of double-stranded DNA in the reaction mixture (as also described in Higuchi et al., Bio/Technology 10:413-417 (1992); Higuchi et al., Bio/Technology 11:1026-1030 (1993); and European Patent Publication Nos. 487,218 and 512,334). The detection of double-stranded target DNA relies on the increased fluorescence that ethidium bromide (EtBr) and other DNA binding labels exhibit when bound to double-stranded DNA. The increase of double-stranded DNA resulting from the synthesis of target sequences results in a detectable increase in fluorescence.

Another method useful for detecting HBV nucleic acid viral markers is through reverse hybridization assays. This is especially useful if a multitude of probes are involved. In one embodiment of the present invention probes that detect multiple HBV nucleic acid viral markers can be examined simultaneously. In one embodiment the selected set of probes are immobilized to a solid support in known distinct locations (dots, lines or other figures). In another embodiment the selected set of probes can be immobilized to a membrane strip in a line fashion. Said probes can be immobilized individually or as mixtures to delineated locations on the solid support. In a specific embodiment, a line probe assay can be used to screen for HBV genotypes containing the specific viral markers of the present invention. The line probe assay involves multiple probes that are immobilized in parallel lines on a membrane, and then a reverse hybridization of amplified nucleic acid fragments is performed. The hybrid can then be detected via a biotin-streptavidin coupling with a non-radioactive color developing system. See, for example, WO 97/40193. Innogentics currently provides a line probe assay for the detection of HBV drug-resistance mutations (INNO-LiPA HBV DRTM kits).

HBV genotyping techniques can also be used to analyze the presence of HBV nucleic acid viral markers. For example, sequence based phylogenetic analysis, differential hybridization, PCR or fragment length polymorphism can be used.

3. Amplification and Detection of HBV Diagnostic Markers

Another aspect of the invention relates to methods for amplifying and detecting the presence of HBV nucleic acid diagnostic markers correlated with therapy failure.

In one embodiment, the invention provides a process for detecting a nucleic acid diagnostic marker correlated with therapy failure wherein a sample suspected of containing an HBV nucleic acid sequence is amplified; the amplified sequence is then contacted with a oligonucleotide probe having a sequence complementary to the nucleotide sequence of the diagnostic marker that is correlated with therapy failure; and the sequence is then detected by hybridizing the probe to the sequence. In one embodiment, amplification is achieved by the use the polymerase chain reaction method.

In another embodiment, the invention provides a process for detecting the presence of nucleic acid diagnostic markers of the HBV from a sample suspected of containing HBV wherein:
(a) the sample is treated under conditions that enable the amplification of a subsequence of HBV genomic nucleic acid that possibly contains the diagnostic marker;
(b) contacting the sample under sequence-specific, stringent hybridization conditions with an oligonucleotide probe that includes a nucleic acid sequence at least 14 nucleotides in length wherein the nucleic acid sequence is complementary to the a section of nucleic acid that carries the codon for the marker;
(c) and detecting hybridization.

DNA or RNA can be extracted from a bodily sample, such as blood, tissue material such as liver by a variety of techniques known in the art. In one specific embodiment, the DNA or RNA is extracted from liver cells. If the sample is impure such as plasma, serum or blood, before amplification it can be treated with an amount of a reagent effective to open the cells, fluids, tissues, viral capsids or animal cell membranes of the sample, and to expose and/or separate the strand(s) of the nucleic acid(s). This lysing and nucleic acid denaturing step to expose and separate the strands will allow amplification to occur much more readily.

The amplification method used can be either polymerase chain reaction (PCR; Saiki et al., 1988), ligase chain reaction (LCR; Landgren et al., Science 241:1077-1080 (1988); Wu &Wallace, Genomics 4:560-569 (1989); Barany, Proc Natl Acad Sci USA 88:189-193 (1991)), nucleic acid sequence-based amplification (NASBA; Guatelli et al., Proc Natl Acad Sci USA 87:1874-1878 (1990); Compton, Nature 350:91-92 (1991)), transcription-based amplification system (TAS; Kwoh et al., Proc Natl Acad Sci USA 86:1173-1177. (1989)), strand displacement amplification (SDA; Duck, Biotechniques 9:142-147 (1990); Walker et al., Proc. Natl. Acad. Sci. USA 89, 392-396 (1992)), or amplification by means of Q9 replicase (Lizardi et al., Nat. Genet. 19:225-232 (1998); Lomeli et al., Clin Chem 35:1826-1831 (1989)) or any other suitable method to amplify nucleic acid molecules known in the art.

Polymerase Chain Reaction

The PCR process for amplification is generally well known in the art (See, for example, U.S. Pat. Nos. 4,683,202 and 4,683,194). The amplification process can involve an enzymatic chain reaction for preparing, in exponential quantities relative to the number of reaction steps involved, a specific nucleic acid sequence, given that the ends of the required sequence are known in sufficient detail that oligonucleotide primers can be synthesized which will hybridize to them, and that a small amount of the sequence is available to initiate the chain reaction. One primer is complementary to the negative (−) strand and the other is complementary to the positive (+) strand. Annealing the primers to denatured nucleic acid followed by extension with an enzyme such as the large fragment of DNA Polymerase I (Klenow) and nucleotides results in newly synthesized (+) and (−) strands containing the target sequence. Because these newly synthesized sequences are also templates for the primers, repeated cycles of denaturing, primer annealing and extension results in exponential accumulation of the region defined by the primer. The product of the chain reaction will be a discrete nucleic acid duplex with termini corresponding to the ends of the specific primers employed.

Any specific nucleic acid sequence can be produced by the present process. It is only necessary that a sufficient number of bases at both ends of the sequence be known in sufficient detail so that two oligonucleotide primers can be prepared which will hybridize to different strands of the desired sequence and at relative positions along the sequence such that an extension product synthesized from one primer, when it is separated from its template (complement), can serve as a template for extension of the other primer into a nucleic acid of defined length. The greater the knowledge about the bases at both ends of the sequence, the greater can be the specificity of the primers for the target nuclei acid sequence, and thus the greater the efficiency of the process. It will be understood that the word primer as used hereinafter can refer to more than one primer, particularly in the case where there is sot ambiguity in the information regarding the terminal sequence(s) of the fragment to be amplified. For instance, in the case where a nucleic acid sequence is inferred from protein sequence information a collection of primers containing sequences representing all possible codon variations based on degeneracy of the genetic code will be used for each strand. One primer from this collection will be substantially conserved with the end of the desired sequence to be amplified.

The specific nucleic acid sequence is produced by using the diagnostic marker nucleic acid containing that sequence as a template. If the target nucleic acid sequence of the sample contains two strands, it is necessary to separate the strands of the nucleic acid before it can be used as the template, either as a separate step or simultaneously with the synthesis of the primer extension products. This strand separation can be accomplished using any suitable denaturing conditions, including physical, chemical or enzymatic means, the word “denaturing” used herein to include all such means. One physical method of separating the strands of the nucleic acid involves heating the nucleic acid unit it is denatured. Typical heat denaturation can involve temperatures ranging from about 80° to 150° C. for times ranging from about 1 to 10 minutes. Strand separation can also be induced by an enzyme from the class of enzymes known as helicases or the enzyme RecA, which has helicase activity and in the presence of riboATP is known to denature DNA. The reaction conditions suitable for separating the strands of nucleic acids with helicases are described by Kuhn Hoffmann-Berling, CSH-Quantitative Biology, 43:63 (1978), and techniques for using RecA are reviewed in C. Radding, Ann. Rev. Genetics, 16:405-37 (1982).

If the original nucleic acid containing the sequence to be amplified is single stranded, its complement is synthesized by adding one or two oligonucleotide primers thereto. If an appropriate single primer is added, a primer extension product is synthesized in the presence of the primer, an agent for polymerization, and the four nucleoside triphosphates described below. The product will be partially complementary to the single-stranded nucleic acid and will hybridize with the nucleic acid strand to form a duplex of unequal length strands that can then be separated into single strands as described above to produce two single separated complementary strands. Alternatively, two appropriate primers can be added to the single-stranded nucleic acid and the reaction carried out.

If the original nucleic acid constitutes the sequence to be amplified, the primer extension product(s) produced will be completely or substantially completely complementary to the strands of the original nucleic acid and will hybridize therewith to form a duplex of equal length strands to be separated into single-stranded molecules.

When the complementary strands of the nucleic acid or acids are separated, whether the nucleic acid was originally double or single stranded, the strands are ready to be used as a template for the synthesis of additional nucleic acid strands. This synthesis is performed under conditions allowing hybridization of primers to templates to occur. Generally it occurs in a buffered aqueous solution, the pH can be in the range of 7-9. A molar excess (for genomic nucleic acid, usually about 108:1 primer:template) of the two oligonucleotide primers can be added to the buffer containing the separated template strands. It is understood, however, that the amount of complementary strand cannot be known if the process herein is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP and TTP are also added to the synthesis mixture, either separately or together with the primers, in adequate amounts and the resulting solution is heated to about 900-1000 C for from about 1 to 10 minutes, alternatively from 1 to 4 minutes. After this heating period the solution is allowed to cool to room temperature, which is preferable for the primer hybridization. To the cooled mixture is added an appropriate agent for effecting the primer extension reaction (called herein “agent for polymerization”), and the reaction is allowed to occur under conditions known in the art. The agent for polymerization can also be added together with the other reagents if it is heat stable. This synthesis reaction can occur at from room temperature up to a temperature above which the agent for polymerization no longer functions. Thus, for example, if DNA polymerase is used as the agent, the temperature is generally no greater than about 40° C. Most conveniently the reaction occurs at room temperature.

The agent for polymerization can be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, polymerase muteins, reverse transcriptase, and other enzymes, including heat-stable enzymes (i.e., those enzymes which perform primer extension after being subjected to temperatures sufficiently elevated to cause denaturation), which will facilitate combination of the nucleosides in the proper manner to form the primer extension products which are complementary to each nucleic acid strand. Generally, the synthesis will be initiated at the 3′ end of each primer and proceed in the 5′ direction along the template strand, until synthesis terminates, producing molecules of different lengths. There can be agents for polymerization, however, which initiate synthesis at the 5′ end and proceed in the other direction, using the same process as described above.

The newly synthesized strand and its complementary nucleic acid strand will form a double-stranded molecule under the hybridizing conditions described above if the target sequence is present, and this hybrid is used in the succeeding steps of the process. In the next step, the sample treated under hybridizing conditions is subjected to denaturing conditions using any of the procedures described above to provide single-stranded molecules if the target sequence is present.

New nucleic acid is synthesized on the single-stranded molecules. Additional agent for polymerization, nucleosides and primers can be added if necessary for the reaction to proceed under the conditions prescribed above. Again, the synthesis will be initiated at one end of each of the oligonucleotide primers and will proceed along the single strands of the template to produce additional nucleic acid. After this step, half of the extension product will consist of the specific nucleic acid sequence bounded by the two primers.

The steps of denaturing and extension product synthesis can be repeated as often as needed to amplify the target nucleic acid sequence to the extent necessary for detection. As will be described in further detail below, the amount of the specific nucleic acid sequence produced will accumulate in an exponential fashion.

When it is desired to produce more than one specific nucleic acid sequence from the first nucleic acid or mixture of nucleic acids, the appropriate numbers of different oligonucleotide primers are utilized. For example, if two different specific nucleic acid sequences are to be produced, four primers are utilized. Two of the primers are specific for one of the specific nucleic acid sequences and the other two primers are specific for the second specific nucleic acid sequence. In this manner, each of the two different specific sequences can be produced exponentially by the present process.

The present invention can be performed in a step-wise fashion where after each step new reagents are added, or simultaneously, where all reagents are added at the initial step, or partially step-wise and partially simultaneous, where fresh reagent is added after a given number of steps. If a method of denaturation, such as heat, is employed which will inactivate the agent for polymerization, as in the case of a heat-labile enzyme, and then it is necessary to replenish the agent after every strand separation step. The simultaneous method can be utilized when an enzymatic means is used for the strand separation step. In the simultaneous procedure, the reaction mixture can contain, in addition to the nucleic acid strand(s) containing the desired sequence, the strand-separating enzyme (e.g., helicase), an appropriate energy source for the strand-separating enzyme, such as rATP, the four nucleoside tri phosphates, the oligonucleotide primers in molar excess, and the agent for polymerization, e.g., Klenow fragment of E. coli DNA polymerase I.

If heat is used for denaturation in a simultaneous process, a heat-stable agent such as a thermostable polymerase can be employed which will operate at an elevated temperature, preferably 50°-105° C. depending on the agent, at which temperature the nucleic acid will consist of single and double strands in equilibrium. For smaller lengths of nucleic acid, lower temperatures of about 40°-50° C. can be employed. The upper temperature will depend on the temperature at which the enzyme will degrade or the temperature above which an insufficient level of primer hybridization will occur. Such a heat-stable enzyme is described, e.g., by A. S. Kaledin et al., Biokhimiya, 45, 644-651 (1980). For this constant temperature reaction to succeed, the primers have their 3′ ends within 6-8 base pairs of each other. Each step of the process will occur sequentially notwithstanding the initial presence of all the reagents. Additional materials can be added as necessary. After the appropriate length of time has passed to produce the desired amount of the specific nucleic acid sequence, the reaction can be halted by inactivating the enzymes in any known manner or separating the components of the reaction.

The amplification can also be carried out using a temperature-cycling reaction wherein the temperature is increased incrementally to allow for extension, annealing and denaturation using a heat-stable enzyme.

The process of the present invention can be conducted continuously. In one embodiment of an automated process, the reaction can be cycled through a denaturing region, a reagent addition region, and a reaction region. In another embodiment, the enzyme used for the synthesis of primer extension products can be immobilized in a column. The other reaction components can be continuously circulated by a pump through the column and a heating coil in series, thus the nucleic acids produced can be repeatedly denatured without inactivating the enzyme.

HBV genotyping using PCR techniques is commonly known in the art. For example, Molecular Informatics Laboratory performs Lamivudine resistant genotyping using Real Time PCR, and Truegene™ performs PCR amplification of the HBV genome. Further, after PCR has been performed on samples suspected of containing HBV, the HBV genome can be sequenced.

II. Detection of HBV Protein/Peptide Markers

In another embodiment, the invention provides a process for detecting viral markers diagnostic for long term lamivudine therapy failure for HBV infection wherein a sample containing HBV protein, peptides, or peptide fragments is analyzed for such viral markers. The proteins, peptides, or peptide fragments correlated with therapy failure can be detected by any generally applicable protein detection technology known in the art, including western blot, two dimensional gel electrophoresis, enzyme linked immunosorbent assays (ELISA), enhanced chemiluminescence (ECL), immunohistochemistry, ELI-Spot assays, peptide sequencing, or antibody based protein array technology. For example, protein expression of HBV viral markers diagnostic for lamivudine treatment can be analyzed with classical immunohistological methods. In these, the specific recognition is provided by the primary antibody (polyclonal or monoclonal) to the specific viral marker, but the secondary detection system can utilize fluorescent, enzyme, or other conjugated secondary antibodies. As a result, an immunohistological staining of the HBV infected tissue section for pathological examination is obtained.

Another method for detecting proteins, peptides, or peptide fragments that are diagnostic for long term response of HBV carriers to lamivudine therapy is the western blot. In brief, a sample containing HBV protein, peptide, or peptide fragments is separated via means of an electrophoretic gel. The separated proteins are then transferred to a medium such as nitrocellulose. Antibodies having a detectable label such as streptavidin-alkaline phosphatase reactive to specific HBV amino acid sequences correlated to lamivudine failure are then contacted onto the nitrocellulose medium containing the HBV amino acid sequences. Reactive antibodies will bind with the corresponding HBV amino acid sequence, and can be detected using a reagent such as nitoblue tetrazolium and 5-bromo-4-chloro-3-indlyl phosphate (BCIP). See, for example, Jalkanen, M., et al., J. Cell. Biol. 101:976-985 (1985); Jalkanen, M., et al., J. Cell. Biol. 105:3087-3096 (1987).

Alternatively, reactive antibodies present in the sera of HBV carriers can be used to detect the presence of HBV viral markers that are diagnostic of lamivudine treatment. Any known antibody assay technique known by those skilled in the art, including enzyme immunoassay (EIA), can be used. For example, in one embodiment a sample from an HBV carrier containing HBV specific antibodies is contacted with a solid support array containing specific HBV peptides correlated with lamivudine therapy success and/or failure. The reactive antibodies are then detected using rabbit anti-human IgG antibodies labeled with streptavidin-alkaline phosphatase and a reagent containing exposed to nitoblue tetrazolium and 5-bromo-4-chloro-3-indlyl phosphate.

Another method for detecting proteins, peptides, or peptide fragments that are diagnostic for long term response of HBV carriers to lamivudine therapy is by sequencing the protein, peptide, or peptide fragment using techniques known to one of ordinary skill in the art (see, for example, Matsudaira, P., J Biol Chem 262: 10035-10038, (1987); Salinovich, O. and Montelano, R., Anal. Biochem. 156: 341, (1986); Tarr, G. E.: Manual Edman Sequencing System. In: Shively, J. E., (ed.) Methods of Protein Microcharacterization. The Humana Press Inc., Clifton, N.J., 1986, pp. 155-194; and Fernandez, J., Andrews, L. and Mische, S., Anal. Biochem. 218: 112-117, (1994)). For example, one could use the Edman technique to determine the amino acid sequence of the peptide. In brief, the Edman chemistry removes amino acid residues from the N-terminus of a protein/peptide, one at a time in sequence. Each cycle of Edman chemistry, needed for the removal of each amino acid residue, consists of three steps: a coupling with phenyl isothiocyanate (PITC) under mildly alkaline conditions to form a phenylthiocarbamyl (PTC)-peptide; cleavage to release the first residue as its anilinothiazolinone (ATZ)-amino acid derivative; conversion of the ATZ derivative to a more stable phenylthiohydantoin (PTH)-amino acid derivative. The PTH-amino acid residue, removed in each cycle of Edman degradation, is identified by small or micro bore RP-HPLC. A full description of the process and possible pitfalls is given by Tarr, G. E.: Manual Edman Sequencing System. In: Shively, J. E., (ed.) Methods of Protein Microcharacterization. The Humana Press Inc., Clifton, N.J., 1986, pp. 155-194. Alternatively, if a sample yields no N-terminal sequence, the N-terminal residue is blocked, degraded during preparative procedures, or for steric reasons unavailable to the Edman chemistry reagents, then the sample can be subjected to controlled specific proteolysis, where the peptides are fractionated and then analyzed. This fractionation approach is described by Fernandez, J., Andrews, L. and Mische, S.: An improved procedure for enzymatic digestion of polyvinylidene difluoride-bound proteins for internal sequence analysis. Anal. Biochem. 218: 112-117, 1994.

III. Arrays

Another aspect of the present invention provides the use of DNA, RNA or peptide arrays to detect HBV nucleic acid viral markers. Such arrays include DNA macroarrays, DNA microarrays, and DNA microchips. DNA arrays, for example, have been described in U.S. Pat. No. 5,837,832, U.S. Pat. No. 5,807,522, U.S. Pat. No. 6,007,987, U.S. Pat. No. 6,110,426, WO 99/05324, 99/05591, WO 00/58516, WO 95/11995, WO 95/35505A1, WO 99/42813, JP10503841T2, GR3030430T3, ES2134481T3, EP804731B1, DE69509925C0, CA2192095AA, AU2862995A1, AU709276B2, AT180570, EP 1066506, and AU 2780499. Such arrays can be incorporated into computerized methods for analyzing hybridization results when the arrays are contacted with prepared sample nucleotides, for example, as described in PCT Publication WO 99/05574, and U.S. Pat. Nos. 5,754,524; 6,228,575; 5,593,839; and 5,856,101. Methods for screening for disease markers are also known to the art, for example, as described in U.S. Pat. Nos. 6,228,586; 6,160,104; 6,083,698; 6,268,398; 6,228,578; and 6,265,174. Further descriptions of DNA array methods can, for example be found in: Shoemaker D. D. et al., Nature 409(6822):922-927 (2001); Kane M. D., et al., Nucleic Acids Res 28(22):4552-7 (2000); Taton T A, et al., Science. 289(5485):1757-60 (2000); Jörg Reichert et al., Anal. Chem., 72(24):6025-6029 (2000); Reinke V, Mol Cell 6(3):605-16 (2000); Marx J. Science 289:1670-1672 (2000); Lockhart D. J. et al., Nature 405(6788):827-836 (2000); Cortese J. D., The Scientist 14[17]:25 (2000); Cortese J. D., The Scientist 14[11]:26 (2000); Fritz J. et al., Science. 288(5464):316-8 (2000); Mark Schena (Ed.), Microarray Biochip Technology, Eaton Publishing Company, Distributed by TeleChem/arrayit.com; Scherf U., et al., Nat Genet. 24(3):236-44 (2000); Ross D. T. et al., Nat Genet. 24(3):227-35 (2000); Walt D. R., Science 287: 451-452 (2000); Afshari C. A. et al., Cancer Res 59(19):4759-60 (1999); Gwynne P. and Page G., Science, 1999 Aug. 6. (special advertising supplement; has a list of microarray-related companies); Baldwin D. et al., Curr Opin Plant Biol 2(2):96-103 (1999); Pollack J. R. et al., Nat Genet 23(1):41-6 (1999); Khan J. et al., Electrophoresis 20(2):223-9 (1999); Gerhold D. et al., Trends Biochem Sci 24(5):168-73 (1999); Ekins R. and Chu F. W., Trends in Biotechnology 17:217-218 (1999); Nuwaysir, E. F. et al., Molecular Carcinogenesis 24:153-159 (1999); Sinclair, B. The Scientist, 13(11):18-20 (1999); The Chipping Forecast, Nature Genetics (January 1999 Supplement); Schena, M. and Davis, R. W. Genes, Genomes and Chips. In DNA Microarrays: A Practical Approach (ed. M. Schena), Oxford University Press, Oxford, UK, 1999; Marton M. J. et al., Nat Med. 4(11):1293-301 (1998); Wang D. G. et al., Science 280(5366):1077-82 (1998); Schena, M. and R. W. Davis. Parallel Analysis with Biological Chips. in PCR Methods Manual (eds. M. Innis, D. Gelfand, J. Sninsky), Academic Press, San Diego, 1998; Lemieux, B. et al., Molecular Breeding 4:277-289 (1998); Schena, M. et al., Trends in Biotechnology 16:301-306 (1998); Service, R. F., Science 282(5388):396-399 (1998); Service, R. F., Science 282(5388):399-401 (1998); Kricka, L., Nature Biotechnology 16:513 (1998); Housman, D., Nature Biotechnology 16(6):492493 (1998); Ramsay, G., Nature Biotechnology 16(1):40-44 (1998); Marshall, A. et al., Nature Biotechnology 16(1):27-31 (1998); Kononen J. et al., Nat. Med. 4(7):844-847 (19998); Blanchard, A. P. (1998) Synthetic DNA Arrays; in Genetic Engineering, Vol. 20, pp. 111-123, edited by J. K. Setlow, Plenum Press, New York; Proudnikov D. et al., Anal Biochem 259(1):34-41 (1998); Chen J. J. et al., Genomics 51(3):313-24 (1998); Wallace R. W., Molecular Medicine Today 3:384-389 (1998); Covacci, A. et al., Drug Development Research 41:180-192 (1997); Forozan, F. et al., Trends in Genetics 13:405409 (1997); Blanchard, A. P. & L. Hood, Nature Biotechnology 14:1649 (1996); Blanchard, A. P. et al., Biosensors & Bioelectronics 11:687-690 (1996); DeRisi J. et al., Nat Genet 14(4):457-60 (1996); Shalon D. et al., Genome Res 6(7):639-45 (1996); Schena M. et al., Proc Natl Acad Sci USA 93(20):10614-9 (1996); and Schena M. et al., Science 270(5235):467-70 (1995).

Probes on an array can be of varying lengths, including, but not limited to, as short as about 10-30 nucleotides long or as long as an entire HBV gene or HBV clone, which can be up to several kilobases. In addition, sequences of the various lengths as those described in FIG. 2 and/or Seq ID Nos. 1-351 can be used as probes. The array can be designed such that all probes on the array can hybridize to their corresponding genes at about the same hybridization stringency. Probes for arrays should be unique at the hybridization stringencies used. A unique probe is only able to hybridize with one type of nucleic acid per target. A probe is not unique if at the hybridization stringency used, it hybridizes with nucleic acids derived from two different genes, i.e. related genes, or non-homologous sequences. The homology of the sequence of the probe to the gene and the hybridization stringency used help determine whether a probe is unique when testing a selected sample. Probes also may not hybridize with different nucleic acids derived from the same gene, i.e., splice variants. Since the splice variants of interest are known as described in Seq ID Nos. 1-351, or, alternatively, in Tables 14-21, several different probe sequences can be chosen from the target gene sequence of interest for an array, such that each probe can only hybridize to nucleic acid derived from one of the splice variants. In one embodiment, arrays containing Seq ID Nos. 1-351 are used at hybridization conditions allowing for selective hybridization. At conditions of selective hybridization, probes hybridize with nucleic acid from only one identified sequence. Alternatively, arrays containing any HBV sequence identified in Tables 14-21 are used at hybridization conditions allowing for selective hybridization. At conditions of selective hybridization, probes hybridize with nucleic acid from only one identified sequence. In another embodiment, arrays containing any HBV sequence of interest are used at hybridization conditions allowing for selective hybridization. At conditions of selective hybridization, probes hybridize with nucleic acid from only one identified sequence.

In one embodiment, the use of the microarray first requires amplification of genes of interest, such as by reverse transcription of mRNA or total RNA followed by polymerase chain reaction using methods known in the art. As the nucleic acid is copied, it is tagged with a label that can be used in the detection and quantitation methods known in the art. The nucleic acid can be labeled with radioactive or non-radioactive labels, but preferably contain fluorescent labels. The labeled nucleic acid is introduced to the microarray containing the sequence probes of interest and allowed to react for a period of time. Thereafter, the substrate is washed free of extraneous materials, leaving the nucleic acids on the target bound to the fixed probe molecules allowing for detection and quantitation by methods known in the art such as by autoradiograph, liquid scintillation counting, and/or fluorescence. As improvements are made in hybridization and detection techniques, they can be readily applied by one of ordinary skill in the art. As is well known in the art, if the probe molecules and target molecules hybridize by forming a strong non-covalent bond between the two molecules, it can be reasonably assumed that the probe and target nucleic acid are essentially completely complementary if the annealing and washing steps are carried out under conditions of high stringency. The detectable label provides a means for determining whether hybridization has occurred. By obtaining an image of the array with a detection and quantitation method known in the art such as autoradiography, liquid scintillation counting, or fluorescence it can be determined if and to what extent HBV gene sequences are present, by comparing intensities at specific locations on the array. High quantitation signals indicate that a particular sequence is present in a prepared sample, and an absent quantitation signal shows that a particular sequence is not present. The presence of various gene sequences under different conditions can be directly compared, such as prior to 3TC treatment and during 3TC treatment. Similarly, it can be determined what sequences are present in response to certain stimuli such as 3TC or other anti-HBV drugs.

In one embodiment, the HBV sequence profile of a patient can be tracked over time using DNA array technologies. In an alternative embodiment, a patient with HBV receiving 3TC as a modality, or other anti-HBV modality, can be monitored, over time, for changes in the aforementioned HBV genomic sequences in response to the treatment.

Arrays containing Seq ID Nos. 1-351, sequences identified in Tables 14-21, or alternatively any identified HBV sequence, of interest, can be made by any array synthesis method known in the art, such as spotting technology or solid phase synthesis via photolithography. Arrays can also be printed on solid substrates, e.g., glass microscope slides. Before printing, slides are prepared to provide a substrate for binding, as known in the art. Arrays can be printed using any printing techniques and machines known in the art. Printing involves placing the probes on the substrate, attaching the probes to the substrate, and blocking the substrate to prevent non-specific hybridization, as known in the art. Preferably the arrays of this invention are synthesized by solid phase synthesis using a combination of photolithography and combinatorial chemistry. Some of the key elements of probe selection and array design are common to the production of all arrays. Strategies to optimize probe hybridization, for example, are invariably included in the process of probe selection. Hybridization under particular pH, salt, and temperature conditions can be optimized by taking into account melting temperatures and by using empirical rules that correlate with desired hybridization behaviors (as described in Keller, G. H., and M. M. Manak (1987) DNA Probes, Stockton Press, New York, N.Y., pp. 169-170, hereby incorporated by reference). Computer models can be used for predicting the intensity and concentration-dependence of probe hybridization.

Moderate to high stringency conditions for hybridization are known in the art. An example of high stringency conditions for a blot are hybridizing at 68° C. in 5×SSC/5× Denhardt's solution/0.1% SDS, and washing in 0.2×SSC/0.1% SDS at room temperature. An example of conditions of moderate stringency are hybridizing at 68° C. in 5×SSC/5× Denhardt's solution/0.1% SDS and washing at 42° C. in 3×SSC. The parameters of temperature and salt concentration can be varied to achieve the desired level of sequence identity between a probe and a target nucleic acid. See, for example, Ausubel et al. (1995) Current Protocols in Molecular Biology, John Wiley & Sons, NY, N.Y., for further guidance on hybridization conditions. The melting temperature is described by the following formula (Beltz, G. A. et al., [1983] Methods of Enzymology, R. Wu, L. Grossman and K. Moldave [Eds.] Academic Press, New York 100:266-285). Tm=81.5° C.+16.6 Log[Na+]+0.41(+G+C)−0.61(% formamide)−600/length of duplex in base pairs.

Nucleic acids useful in this invention can be created by Polymerase Chain Reaction (PCR) amplification. PCR products can be confirmed by agarose gel electrophoresis. PCR is a repetitive, enzymatic, primed synthesis of a nucleic acid sequence. This procedure is well known and commonly used by those skilled in this art (see, for example, Mullis, U.S. Pat. Nos. 4,683,195, 4,683,202, and 4,800,159; Saiki et al., Science 230:1350-1354 (1985)). PCR is used to enzymatically amplify a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Since the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes that can be used are known to those skilled in the art.

Alternatively, probes made of peptide nucleic acids (PNAs) can be used as substitutes for probes made of oligonucleotides for the same uses as described above. The substitution of PNAs for oligonucleotides is well known in the art: The synthesis of peptide nucleic acids via preformed monomers has been described, for example, in PCT patent applications WO 92/20702 and WO 92/20703. Recent advances have also been reported on the synthesis, structure, biological properties, and uses of PNAs. See, for example, PCT Patent application WO 93/12129, U.S. Pat. No. 6,617,422 to Neilsen P. E. et al., U.S. Pat. No. 5,539,083 to Cook et al., U.S. Patent application US20030059789A1, U.S. Pat. No. 6,475,721 to Kleiber et al., Egholm et al., Nature:365, 566-568 (1993), Nielsen et al., Science 254:1497-1500 (1991); and Egholm et al., J. Am. Chem. Soc., 114:1895-1897 (1992).

IV. Kits

In another embodiment, the invention provides a kit for the detection of a marker of resistance to long term lamivudine treatment of hepatitis B virus, and in particular, an HbeAg negative virus. The kit can contain a compartment which contains an oligonucleotide probe which binds substantially to a nucleic acid subsequence of the HBV that contains the diagnostic marker. Alternatively, the kit contains peptide nucleic acid (PNA) or other antisense mimic probe in substitution for the oligonucleotide. The kit can also contain reagents to detect the hybridization of the probe to the HBV nucleic acid viral marker. The present invention also includes kits that can contain a primer for the PCR amplification of HBV nucleic acids. A kit can also contain a means for detecting amplified HBV nucleic acids, such as an oligonucleotide or peptide nucleic acid probe. In some cases, the probe is fixed to an appropriate support membrane. Other optional components of the kit include, for example, an agent to catalyze the synthesis of primer extension products, the substrate nucleoside triphosphates, means used to label (for example, an avidin-enzyme conjugate and enzyme substrate and chromogen if the label is biotin), the appropriate buffers for PCR or hybridization reactions, and instructions for carrying out the present method.

In addition, the kit can have a container which includes a positive control containing one or more nucleic acids with a sequence of the HBV viral genome correlated with therapy failure and/or a container including a negative control without such nucleic acids. Moreover, the kit can have a container for a restriction enzyme capable of cleaving a nucleic acid containing the target sequence at a site contained in a sequence in the probe.

The invention also provides a kit for the detection and/or genetic analysis of one or more viral markers of HBV that are correlated with therapy failure that can be present in a biological sample comprising the following components: (i) when appropriate, a means for releasing, isolating or concentrating the nucleic acids present in the sample; (ii) when appropriate, at least one suitable primer pair; (iii) at least two probes as defined above, possibly fixed to a solid support; (iv) a hybridization buffer, or components necessary for producing said buffer; (v) a wash solution, or components necessary for producing said solution; (vi) when appropriate, a means for detecting the hybrids resulting from the preceding hybridization; (vii) when appropriate, a means for attaching said probe to a known location on solid support; and/or (viii) instructions for carrying out the present method.

Furthermore, the invention also provides for a kit that contains peptide or peptide fragments corresponding to viral markers correlated with lamivudine therapy failure that can be used in an immunoassay to detect the presence of reactive antibodies in a sample. The peptide can be in a stabilized solution or lypholized. Such kit can contain an appropriate solution for hydrolyzing a lyophilized peptide. The kit can also contain an appropriate solid medium for blotting the aforementioned peptide on. The kit can also contain an appropriate reagent for detecting the presence of reactive antibodies to the peptide, such as an anti-human IgG antibody labeled with streptavidin-alkaline phosphatase. Furthermore, the kit can contain a detection agent such as nitoblue tetrazolium and 5-bromo-4-chloro-3-indlyl phosphate (BCIP).

Alternatively, the kit can contain antibodies reactive to specific peptide sequences associated with lamivudine therapy.

The following examples illustrate various embodiments of the invention and are not intended to be limiting in any respect.

### EXAMPLES

### Example 1

### Changes in Polymerase Sequences Before and During Treatment

Patients and Methods

Patients

Twenty-six patients with HBe-negative chronic hepatitis B, who received lamivudine therapy at the Department of Gastroenterology, Molinette Hospital in Turin, Italy, were consecutively enrolled: 24 men and 2 women, with a mean age of 47.3 years ranging between 21-57 years. All patients had detectable HBV DNA by polymerase chain reaction (PCR) assay (HBV Monitor, Roche Diagnostics, Inc.) and by solution hybridization assay (Abbott Diagnostics, Inc.) at baseline and for at least 6 months before therapy. All patients had elevated serum alanine transaminase (ALT) levels (concentrations>1.5× upper limit of normal, 35 IU/L) on at least 3 separate routine monthly tests during the year before lamivudine treatment. Patients were negative for hepatitis C, hepatitis D, or human immunodeficiency virus antibodies, and there were no evidences of autoimmune hepatitis. None of the patients were treated with interferon or other antiviral agents, such as famciclovir or adefovir dipovoxil, during the study or within six months before the beginning of lamivudine treatment.

Immediately before treatment, or during 6 months before enrollment, all patients underwent liver biopsy. All patients showed histological findings of chronic hepatitis or early stage of cirrhosis (class A Child-Pugh score).

Patients received lamivudine orally at a daily dose of 100 mg; the mean duration of treatment was 38 months (range 27-53 months). All patients were followed with monthly clinical examination and routine laboratory tests.

The local ethics committee approved the study and all patients gave written informed consent before screening. The study was conducted in accordance with the ethical guidelines of the Declaration of Helsinki (1975).

Methods

Blood test—An aliquot of serum was collected at each visit and stored at −70° C. until used for analysis.

Pretreatment samples from all patients and subsequent samples that were HBV-DNA positive by PCR were examined for HBV precore/core promoter and HBV polymerase gene sequence mutations.

HBV serologic markers were tested by commercial enzyme immunoassay kits: HBeAg and anti-HBeAg (DiaSorin, Italy), and HBsAg (Abbott Laboratories, Inc.). Serum samples were also evaluated for routine biochemical markers of liver disease, including ALT.

Serum HBV DNA for the evaluation of response to treatment was analyzed by the HBV Monitor™ assay (Roche Diagnostic, Inc.) with a detection limit of 100 copies/ml, and a solution hybridization assay (Abbott Laboratories, Inc.) with a detection limit of 10 pg/ml.

DNA extraction, PCR amplification and determination of core promoter, precore and polymerase region sequences

HBV DNA was extracted from 50 μl of serum. Briefly, serum was incubated for protein digestion in lysis buffer (100 mmol/L Tris-HCl (pH 8.0), 50 mmol/L ethylendiaminetetraacetic acid EDTA, 4% sodium dodecyl sulfate, 400 mmol/L NaCl, containing 20 mg/mL proteinase K) overnight at 37° C. (Smedile, et al., 1986. Hepatol. 6:1297-1302). After incubation, DNA was extracted with phenol/chloroform/isopropanol, precipitated by ethanol and resuspended in distilled water. During the extraction procedure, standard precautions were taken to prevent cross-contamination. Moreover, negative serum samples were included in each test, and at each step.

Amplification was performed by an in-house nested polymerase chain reaction assay. For the first round of PCR amplification, 5 μl of resuspended patient sample DNA was used in a final volume of 35 μl containing 20 mmol/L Tris-HCl (pH 8.3), 100 mmol/L KCl, 15 mmol/L MgCl2, 100 mol/L of deoxynucleotide mix, 0.5 U of Taq polymerase (Roche) and 20 pmol of each primer. The reaction was performed for 35 cycles of 94° C. for 1 minute, 53° C. for 1 minute for the precore/core region or 51° C. for the polymerase region, and 72° C. for 2 minutes, with an extension of 7 minutes at 72° C. at the end. For the second round PCR amplification, 1 μl of PCR product from the first reaction was added to 49 μl of the same PCR mixture and was amplified under the same conditions except the annealing temperature was 41° C. for precore/core region or 48° C. for the polymerase region was used.

The following primers were used for the amplification of a 342 nucleotide sequence of the HBV precore/core region: 5′-CTTCGCTTCACCTCTGCACG-3′ and 5′-ACCACCCACCCAGGTAGCTA-3′ for the first PCR amplification, and 5′-TTACATAAGAGGACTCTTGG-3′ and 5′-AGAAGATCTCGTACTGAAGG-3′ for the second round PCR. For amplification of a 649 nucleotide sequence of the HBV polymerase gene, including all five active polymerase domains (A-E), the first round of PCR was performed using primers 5′-CAGACTTTCCAATCAATAGGCC-3′ and 5′-AGCAAAACCCAAAAGACCCA-3′, and the second round of PCR amplification using 5′-TMTCTAGGGGGAACTACCGT-3′ and 5′-GATGTGATCTTGTGGCAATG-3′.

PCR products were analyzed by gel electrophoresis in 1.5% agarose gel stained with ethidium bromide and photographed under ultraviolet light. PCR products were purified by the Quick-Step 2 Purification Kit (Edge BioSystem, Gaithersburg, Md. USA), according to the manufacturer's instruction, and then directly sequenced (MWG Biotech, Inc., High Point, N.C. USA). Sequencing was performed on both DNA stands and sequencing chromatograms were examined for heterogeneity at critical nucleotide positions.

Pretreatment serum samples were also analyzed for mutations in the HBV polymerase associated with lamivudine resistance (LI 80M, M204V/I) Allen, et al, Hepatology 27:1670-1677 (1998); Stuyver, et al. Hepatology 33:751-757 (2001)) by INNO-LiPA HBV DRTM analysis (Stuyver, et al, J. Clin. Microbiol. 38:702-707 (2000)) (Innogenetics, Inc., Ghent, Belgium) according to the manufacturer's instructions. Serum samples obtained at the time of HBV DNA reappearance (breakthrough) during treatment were also examined by the LiPA strip test for HBV drug-resistance mutations.

HBV Genotyping

HBV genotyping was determined at baseline and on every occasion of PCR positive sample by comparing the obtained S gene nucleotide sequences with published sequences. (Norder H, Courece A M, Magnius L O. Complete genomes, phylogenetic relatedness and structural proteins of six strains of hepatitis B virus, four of which represent two new genotypes. Virology 198:489-503 (1994)).

Definitions—“Responder” patients were classified as attaining both a biochemical and virological response: normalization of ALT values and the absence of serum HBV DNA by Monitor™ PCR in two consecutive determinations during treatment. “Breakthrough” was defined as those patients in who HBV DNA reappeared by Monitor™ PCR assay after an initial antiviral response (serum HBV DNA negative by Monitor™ PCR), eventually associated with an increase of ALT levels greater than 1.5× the upper limit of normal (35 IU/L). “Non-responder” patients were those who failed to attain normal ALT levels and failed to clear serum HBVDNA by Monitor PCR in any determinations during therapy. Although the in-house nested PCR assay used for sequence analysis was found to be 10 to 15-fold more sensitive than the Monitor™ PCR assay, HBV DNA reactivity by Monitor™ PCR was used to define clinical response as this test is qualified for quantitative HBV DNA analysis of clinical serum samples.

Statistical analysis—Statistical analyses (Mann-Whitney Rank Sum, 2-tailed t-test, Fisher's exact test) were perdormed using Minitab™ (Minitab, Inc., College Park, Pa. USA).

Nomenclature—The amino acid positions for the HBV polymerase gene (HBpol) are numbered to be consistent with the newly established scheme (Lok A. S. et al., J. Clin. Microbiol. 40: 3729-3734 (1990)) designed to standardize the nomenclature for lamivudine-resistance mutations, pL180M and pM204V/1 (originally designated as pL528M or pL526M, and pM552V/I or pM550V/I).

Results

Virological and Biochemical Response

Prior to treatment (baseline), all 26 patients were HBV-DNA positive (mean 2.7×106 copies/ml, median 1.0×106 copies/ml, range 0.001-9.6×106 copies/ml), and had elevated ALT levels (mean 219 IU/L, median 173 IU/L, range 62-953 IU/L; upper limit of normal=35 IU/L) (Tables 14 and 15). Twenty-two of 26 patients (85%) were HBeAg negative and anti-HBeAg positive in sera collected at baseline and during treatment (Table 15).

Table 15 shows the DNA sequences that represent the latest bi-annual analysis during therapy (minimum of 24 months) (although DNA sequence analysis was performed at multiple time points). No nucleotide heterogeneity was observed at any of the positions discussed in any of the patient samples. In Table 15, R is a responder; NR is a non-responder; BK is a breakthrough. Also, in Table 15, the “*” indicates sequence not determined since samples were PCR negative (‘ud’, undetectable, limit of detection 1000 copies/ml); the “#” indicates transient detection of M2041; the “ND” indicates not detected; the “˜” indicates transient detection of wild-type; the “\hat{ }” indicates detection of wild-type at 18 months; and the “&” indicates transient detection of G 896A mutation.

Two patients (#1, #4) were HBeAg/anti-HBeAg negative, and one (#23) was HBeAg positive/anti-HBe negative. One patient (#3) was HBeAg/anti-HBe positive at baseline and became anti-HBe negative after 12 months of therapy.

By week 24 of treatment, HBV DNA became undetectable in 19 patients (73%), and serum ALT normalized in 13 (50%). After a minimum of 27 months of therapy, seven patients (27%) were responders, 12 (46%) were breakthroughs, and 7 (27%) were non-responders (Table 15). Virologic breakthrough was observed after 12 months of therapy in 6 patients, between 12 and 18 months in 5 patients, and after 38 months in one patient (#26). In the 7 non-responder patients, HBV-DNA concentrations and ALT levels were generally lower than baseline (but always detectable) for each patient during the first 6 to 12 months of therapy (data not shown), but then increased, in nearly all cases becoming equal to or greater than baseline levels (Table 15). Following identification of long-term treatment outcome, an analysis of the pretreatment serum HBV DNA and ALT values revealed that there were no significant differences in pretreatment serum HBV DNA or ALT levels, or the duration of therapy among the three response categories (Table 14).

Changes in HBV Polymerase Gene Sequences Before and During Treatment.

The amino acid positions for the HBV polymerase gene (HBpol) are numbered to be consistent with the newly established scheme designed to standardize the nomenclature for lamivudine-resistance mutations, L180M and M204V/I (Stuyver, et al., Hepatology 33: 751-757 (2001))(originally designated as L528M or L526M, and M552V/I [Allen, et al., Hepatology 27:1670-1677 (1998); Pillay, et al., International Antiviral News 6(9): 167-169 (1998)]).

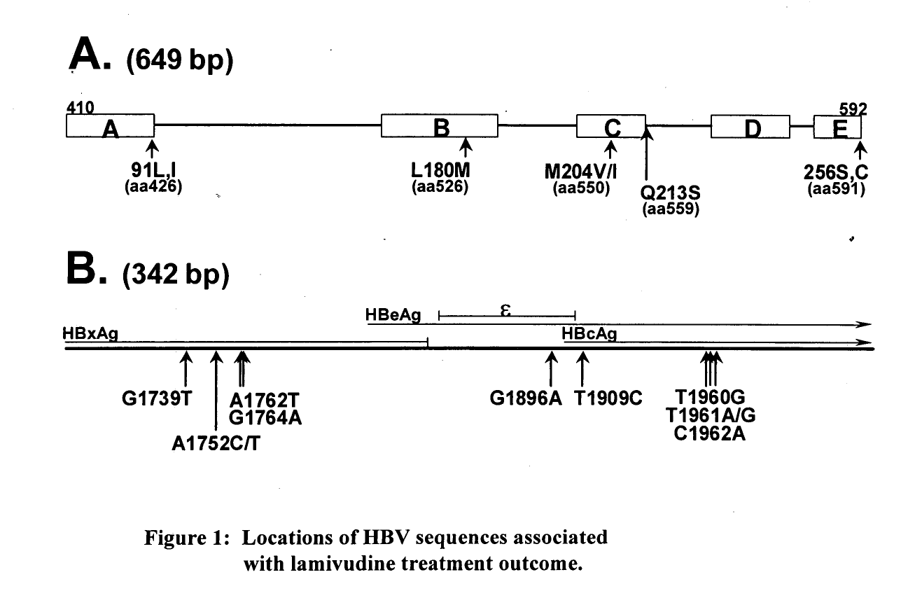

At baseline, no patients carried detectable mutations in the HBV polymerase associated with lamivudine resistance (pL180M, pM204V/I) by DNA sequence analysis (Table 15, FIG. 1) and by INNO-LiPATM analysis (see Stuyver et al., Hepatol 33:751-757 (2001), and Lok A. S. et al., J. Clin. Microbiol 40:3729-3734 (2002)). Alignment of HBsAg gene sequences (see Lok A. S. et al., J. Clin Microbiol 40:3729-3734 (2002)) in serum samples collected at baseline revealed that 25 of 26 patients were HBV genotype D, and one (#4) was genotype A. No changes in genotype occurred in these patients during treatment.

During treatment, 6 patients (4 breakthroughs, 2 non-responders) acquired pL180M (Table 15). Five of these 6 patients (4 breakthroughs, 1 non-responder) also developed pM204V, and one (#22) acquired pM204I. Five additional patients (3 breakthroughs, 2 non-responders) developed pM204I after 12 months of therapy (Table 15). One breakthrough patient (#24) acquired pM204I after 18 months of treatment and was wild-type at p180L, but after 24 months of therapy, a change to pM204V and pL180M was observed. In this study, pM204V/I and pL180M were not detected in 5 of the 12 patients (40%) experiencing virologic breakthrough during lamivudine therapy (Table 15). No sequence heterogeneity was observed at these positions, or at any of the other positions discussed in any of the patient samples.

Two naturally-occurring DNA polymorphisms HBVpol in pretreatment serum samples were found to be significantly (p<0.03 to <0.0003) correlated with the failure (breakthrough or non-response) of long-term response to lamivudine therapy (Tables 17 and 18, FIG. 1).

Table 17 shows the DNA sequences that represent the latest bi-annual analysis during therapy (minimum of 24 months) (although DNA sequence analysis was performed at multiple time points). No nucleotide heterogeneity was observed at any of the positions discussed in any of the patient samples. In Table 17, R is a responder; NR is a non-responder; BK is a breakthrough. Also, in Table 17, “*” indicates sequence not determined since samples were PCR negative; the “ ” indicates ATT 91I, CTT 91L; the “#” indicates AGT 256S, TGT 256C; and the “\hat{ }” indicates A1750T instead of A1752C/T.

One polymorphism was the presence of either a leucine or isoleucine at amino acid 91 of HBpol (p91L or p91I, respectively). P91L was present in 16 of 26 patients (61%) (11/12 breakthroughs, 5/7 non-responders), and was maintained in 15 of these 16 patients during treatment. The remaining 10 patients (1/12 breakthrough, 2/7 non-responder, 7/7 responders) carried p91I. A change from p91I to p91L occurred in one breakthrough patient (#13) at month 11 of treatment (the first sample in which the nested PCR reaction could detect HBV DNA during therapy), one month before virological rebound (as defined by the Monitor™ PCR assay) and the emergence of pM204I.

The other pretreatment DNA sequence polymorphism in HBpol correlated with long-term treatment response was a cysteine or serine at amino acid 256 of HBpol (p256C or p256S, respectively). p256C was present in 13 of 26 patients (50%) (4/7 non-responders, 9/12 breakthroughs), and was sustained in 11 of these patients during therapy (4 non-responders, 7 breakthroughs). HBV in the remaining 13 patients (3/12 breakthrough, 3/7 non-responder, 7/7 responders) was p256S. All of the 13 patients carrying p256C at baseline also carried p91L. The breakthrough patient (#13), who switched from p911 to p91L during therapy, maintained p256S before and during therapy (Tables 17 and 18). One breakthrough patient (#4), switched from p91L to p91I and from p256C to p256S during therapy, and carried wild-type sequences at p180L and p204M throughout treatment (Tables 17, 18). During therapy, another breakthrough patient (#5), switched from p256C (observed at baseline) to p256S, maintained p91L (observed at baseline), and acquired pM204I (Tables 17, 18).

A cluster of previously unreported mutations was detected in HBpol in the pretreatment sera of 7 patients (5 breakthrough, 2 non-responder), producing a glycine to serine substitution (pQ213S) (Tables 17 and 18, FIG. 1). These mutations were maintained during therapy in all 7 patients. In all 7 patients, all three nucleotides in this codon were changed (CAA→TCG). Six of these 7 patients also acquired mutations in the YMDD motif during therapy. None of the 7 responder patients carried this cluster of mutations at baseline or during therapy. However, mutations at this codon were not statistically correlated with treatment response (Table 18).

Published reports have described several changes in HBpol other than those discussed above in individual patients exhibiting HBV resistance to lamivudine (see Naoumov N. V. et al., Hepatol 24:282A (1996); Torresi J. et al., Virol 299:88-99 (2002); Chayama K. et al., Hepatol 27: 1711-1716 (1998); Bartholomew M. M. et al., Lancet 349:20-22 (1997); Fu L et al., Bioch Pharmacol 55:1567-1572 (1998); Pillay D. et al., Int Antivir News 6:167-169 (1998); Yeh C. T. et al., Hepatol 31:1318-1326 (2003); Kao J. H. et al., J Med Virol 68:216-220 (2002); Sallam T. A. et al., J Med Virol 68:328-334 (2002)). Only a few of these previously described mutations were observed in the patients. All were present at baseline and were maintained during therapy. Q130P (Q478R) was present in all 26 patients, S211N (S559N) and S219A (S567A) were observed in 4 patients, L82M (L430M) and L229M/V (L577M/V) were present in a non-responder (#11), and Q215H was found in one breakthrough patient (#13).

In addition to the sequence changes noted above, additional sequence changes to the HBV polymerase gene were noted in the Lamivudine treated patients (Tables 19a and 19b). Additional Polymerase and S gene nucleic acid substitutions appear in Table 21.

### Example 2

### Changes in HBV Precore/Core Sequences Before and During Treatment

A total of 22 of 26 patients carried HBV with mutations conferring an HBe-negative genotype (Tables 15, 16). DNA sequence analysis of the precore region in sera collected at baseline showed that the precore stop codon mutation (G1896A) was present in 16 of 26 patients (62%) (8 breakthroughs, 4 non-responders, 4 responders) (Tables 15 and 16, FIG. 1). All of these 16 patients were HBeAg negative and anti-HBeAg positive. After a minimum of 27 months of treatment, HBV DNA was detectable in 14 of these 16 patients. Mutations in 4 of these 14 patients (29%) (3 breakthrough, 1 non-responder) reverted to wild-type at these positions (Table 15). Two (#6, #24) of the 10 patients (20%) carrying virus wild-type at this position at baseline, acquired G1896A after 12 months of therapy (Table 15).

DNA sequence analysis of the core promoter region in baseline sera showed that 16 of the 26 patients (61%) (8 breakthroughs, 4 non-responders, 4 responders) carried the double promoter mutation, A1762T/G1764A, at baseline (Tables 15 and 16, FIG. 1). Eleven of these 16 patients (69%) also carried G1896A at baseline. After a minimum of 27 months of treatment, HBV DNA was detectable in 12 of these 16 patients. Three of these 12 patients (25%) (all breakthrough) reverted to wild-type (Table 15). After 12 months of therapy, one non-responder patient (#9) who harbored wild-type virus sequence at nucleotide 1764 at baseline acquired G1764A. In a breakthrough patient (#17), A1762T/G1764A present at baseline were replaced by wild-type sequences after 12 months of treatment, but by 18 months of therapy, these mutations re-emerged in the dominant circulating HBV. Reversion at either G1896A or A1762T/G1764A was not correlated with the acquisition of established mutations in the HBV polymerase B or C domains that confer resistance to lamivudine (Tables 15, 16) in this study.

Several mutations were identified in the precore/core region that were never observed in any of the 7 responder patients (Tables 17 and 18, FIG. 1). One or more of these mutations were collectively observed in pretreatment sera from 10 patients (5/7 non-responder, 5/12 breakthrough), and in two additional breakthrough patients during therapy. When taken collectively, this group of 8 precore/core promoter region mutations was significantly (p<0.03) correlated with long-term treatment failure (Table 18).

Two mutations in the HBV precore region recently associated with occult HBV infections (T1802C, T1803G), were present in all 26 patients in the cohort in the pretreatment serum samples. A polymorphism of cytosine or thymidine at nucleotide 1733 in the core promoter region of HBV previously described in HBe-negative chronic infections (see Sallam T. A. et al., J Med Virol 68:328-334 (2002)), was found in 22 of 26 patients (11 breakthrough, 7 non-responders, 4 responders) in the pretreatment samples. However, none of the patients in the cohort carried either the multi-base substitution or deletion at nucleotides 1753-1764 that were associated with this polymorphism.

In addition to the sequence changes above, additional precore/core sequence changes were noted in Lamivudine treated patients (Tables 20a and 20b). Additional HBV Core and X gene nucleic acid substitutions appear in Table 22.

TABLE 14
Attributes of the three categories of treatment responses.

| Initial Serum | | | | | | | | Duration | | |
|---|---|---|---|---|---|---|---|---|---|---|
| HBV DNA (log₁₀)# | | | | | Initial ALT (IU/L)# | | | of treatment (m .)# | | |
| Outcome | No. | Mean | Median | Range | Mean | Median | Range | Mean | Median | Range |
| responder | 7 | 6.43 | 6.00 | 3.00-6.98 | 309 | 245 | 65-953 | 34 | 28 | 27-50 |
| non- | 7 | 5.48 | 4.03 | 3.00-6.00 | 207 | 245 | 66-295 | 38 | 38 | 33-47 |
| responder | | | | | | | | | | |
| Breakthrough | 12 | 6.30 | 4.61 | 3.00-7.02 | 172 | 128 | 62-715 | 40 | 38 | 27-53* |
| All patients | 26 | 6.23 | 4.86 | 3.00-7.02 | 219 | 173 | 62-953 | 38 | 38 | 27-53 |

#indicates mean and median values for the duration of group treatment periods, initial HBV serum HBV DNA, and mean initial ALT values were not significantly different among the groups (Mann-Whitney rank sum). Median initial ALT for the breakthrough group was significantly different from the responder and non-responder groups by Mann-Whitney (p = .047), but was not significantly different by two-sample t-test (p = .29).
*indicates the 9/12 patients that experienced breakthrough by 21 months [(mo. 12 (1 pt.), mo. 18(5 pts.), mo. 21(3 pts.)]. The remaining 3 patients experienced breakthrough at months 24, 27 and 38.

TABLE 15
HBV core/precore and drug-resistant polymerase sequences before and during treatment

| | PRETREATMENT | | | | | | | | | | | |
|---|---|---|---|---|---|---|---|---|---|---|---|---|
| | | | | | HBV-DNA | | | PPRECORE/CORE | | | POLYMERASE | |
| | Outcome | Pt # | Sex | ALT (IU/L) | (log₁₀/ml) | HBeAg | Anti-HBe | 1762 | 1764 | 1896 | L180 M | M204V/I |
| | | | | | | | | | | | CTG | |
| Cons. seq | | | | | | | | A | G | G | TTG | ATG |
| | R | 1 | M | 953 | 6.00 | − | − | A | G | G | CTG | ATG |
| | R | 2 | M | 190 | 6.48 | − | + | T | A | A | CTG | ATG |
| | R | 7 | F | 262 | 3.00 | − | + | T | A | G | TTG | ATG |
| | R | 14 | M | 187 | 6.72 | − | + | A | G | A | CTG | ATG |
| | R | 18 | M | 65 | 6.98 | − | + | T | A | A | CTG | ATG |
| | R | 23 | M | 245 | 3.40 | + | − | A | G | G | TTG | ATG |
| | R | 25 | M | 262 | 5.00 | − | + | T | A | A | CTG | ATG |
| | NR | 3 | M | 245 | 3.00 | + | + | T | A | G | CTG | ATG |
| | NR | 6 | M | 135 | 4.03 | − | + | T | A | G | TTG | ATG |
| | NR | 9 | M | 66 | 3.73 | − | + | G | G | G | CTG | ATG |
| | NR | 11 | M | 286 | 5.00 | − | + | A | G | A | CTG | ATG |
| | NR | 15 | M | 295 | 6.00 | − | + | T | A | A | CTG | ATG |
| | NR | 21 | M | 248 | 4.00 | − | + | T | A | A | CTG | ATG |
| | NR | 22 | M | 174 | 6.00 | − | + | A | G | A | CTG | ATG |
| | BK | 4 | M | 62 | 3.90 | − | − | T | A | G | CTG | ATG |
| | BK | 5 | F | 194 | 7.00 | − | + | T | A | A | CTG | ATG |
| | BK | 8 | M | 171 | 7.02 | − | + | T | A | A | CTG | ATG |
| | BK | 10 | M | 715 | 3.00 | − | + | T | A | A | CTG | ATG |
| | BK | 12 | M | 128 | 4.66 | − | + | A | G | G | CTG | ATG |
| | BK | 13 | M | 84 | 3.00 | − | + | A | A | A | CTG | ATG |
| | BK | 16 | M | 128 | 6.00 | − | + | T | A | A | CTG | ATG |
| | BK | 17 | M | 136 | 4.56 | − | + | T | A | A | CTG | ATG |
| | BK | 19 | M | 155 | 6.08 | − | + | T | A | A | CTG | ATG |
| | BK | 20 | M | 94 | 3.00 | − | + | A | G | A | CTG | ATG |
| | BK | 24 | M | 86 | 3.00 | − | + | T | A | G | CTG | ATG |
| | BK | 26 | M | 115 | 6.00 | − | + | A | A | G | CTG | ATG |

| | TREATMENT (LAST FOLLOW UP) | | | | | | | | | |
|---|---|---|---|---|---|---|---|---|---|---|
| | Treatment | | | | | PPRECORE/ | | | | |
| | duration | | HBV DNA | | | CORE | | | POLYMERASE | |
| | (months) | ALT (IU/L) | (log₁₀/ml) | HBeAg | Anti-HBe | 1762 | 1764 | 1896 | L180 M | M204V/I |
| Cons. seq | | | | | | A | G | G | CTG | ATG |
| | 29 | 30 | ud* | − | − | * | * | * | *** | *** |
| | 27 | 24 | ud | − | + | * | * | * | *** | *** |
| | 28 | 20 | ud | − | + | * | * | * | *** | *** |
| | 27 | 18 | ud | − | + | * | * | * | *** | *** |
| | 27 | 24 | ud | − | + | * | * | * | *** | *** |
| | 50 | 21 | ud | − | + | * | * | * | *** | *** |
| | 47 | 16 | ud | − | + | * | * | * | *** | *** |
| | 39 | 68 | 4.25 | + | − | T | A | G | CTG | ATC |
| | 33 | 37 | 4.55 | − | + | A | A | A | TTG | ATG |
| | | 17 | | | | | | | | |
| | 35 | 4 | 6.11 | − | + | G | A | G | CTG | ATG |
| | 37 | 66 | 7.28 | − | + | A | G | A | CTG | ATT |
| | 38 | 74 | 5.19 | − | + | T | A | A | CTG | ATG |
| | | 12 | | | | | | | | |
| | 47 | 4 | 6.19 | − | + | T | A | A | ATG | GTG |
| | 39 | 85 | 6.11 | − | + | A | G | A | ATG | ATT |
| | | 16 | | | | | | | | |
| | 50 | 9 | 5.00 | − | − | T | A | G^{&} | CTG | ATG |
| | | 10 | | | | | | | | |
| | 53 | 5 | 5.31 | − | + | T | A | A | CTG | ATT |
| | 39 | 21 | 5.17 | − | + | A^{\hat{ }} | G | G^{\hat{ }} | ATG | GTG |
| | 45 | 75 | 6.20 | − | + | A | G | A | ATG | GTG |
| | 33 | 81 | 5.67 | − | + | A | G | G | ATG | GTG |
| | 33 | 51 | 6.62 | − | + | A | G | A | CTG | ATT |
| | | 20 | | | | | | | | |
| | 38 | 5 | 5.88 | − | + | T | A | A | CTG | ATG |
| | 36 | 80 | 4.54 | − | + | T^{˜} | A | A^{˜} | CTG | ATG |
| | | 11 | | | | | | | | |
| | 31 | 2 | 6.08 | − | + | T | A | A | CTG | ATT |
| | | 11 | | | | | | | | |
| | 52 | 4 | 7.02 | − | + | A | G | G | CTG | ATG |
| | | 15 | | | | | | | | |
| | 38 | 5 | 6.18 | − | + | T | A | A | ATG | GTG^{#} |
| | | 71 | | | | | | | | |
| | 27 | 9 | 5.13 | − | + | A | A | G | CTG | ATG |

TABLE 16
Relationships of precore/core and polymerase region mutations.

| | | NO. OF PATIENTS WITH | | | |
|---|---|---|---|---|---|
| | No. of | ALT | Sustained ALT | PC/C | YMDD |
| | Patients (%) | elevation | Normalization | Mutations^{˜} | Mutations^{#} |
| PRETREATMENT (26 pts) | | | | | |
| A1762T/G1764A only | 5 (19%) | 5 | 0 | | 0 |
| A1762T/G1764A + G1896A | 11 (42%) | 11 | 0 | | 0 |
| G1896A only | 5 (19%) | 5 | 0 | | 0 |
| Wild-type precore/core | 5 (19%) | 5 | 0 | | 0 |
| Wild-type p204M | 26 (100%) | 26 | 0 | 21 | |
| M204I (YIDD) | 0 | 0 | 0 | 0 | |
| M204V (YVDD) | 0 | 0 | 0 | 0 | |
| TREATMENT (18 pts)* | | | | | |
| A1762T/G1764A only | 2 (11%) | 1 | 1 | | 1 |
| A1762T/G1764A + G1896A | 6 (33%) | 4 | 1 | | 3 |
| G1896A only | 5 (28%) | 4 | 0 | | 4 |
| Wild-type precore/core | 5 (28%) | 3 | 0 | | 2 |
| Wild-type p204M | 8 (44%) | 5 | 3 | 5 | |
| M204I (YIDD) | 5 (28%) | 3 | 2 | 5 | |
| M204V (YVDD) | 5 (28%) | 4 | 1 | 3 | |
| OUTCOME SUMMARY | | | | | |
| (26 pts) | | | | | |
| Responder | 7 (27%) | 7 | 0 | * | * |
| Non-responder | 7 (27%) | 7 | 0 | 6 | 4 |
| Breakthrough | 12 (46%) | 5 | 7 | 6 | 7 |

*indicates sequences from responder patients that were not analyzed since samples were HBV DNA negative.
^{˜}includes A1762T, G1764A, and G1896A.
^{#}includes M204V and M204I.

TABLE 17
Newly identified HBV sequences in pretreatment serum potentially correlating with treatment response.

| PRETREATMENT | | | | | | | | | TREATMENT (LAST FOLLOW UP) | | | | | | |
|---|---|---|---|---|---|---|---|---|---|---|---|---|---|---|---|
| PRECORE/CORE | | | | | | POLYMERASE | | | | | | | POLYMERASE | | |
| Out | | | | | | | Q | S/C | PECORE/CORE | | | | | | S/C |
| come | Pt # | 1739 | 1752 | 1909 | 1960-62 | 91 I/L^{˜} | 213 S | 256^{#} | 1739 | 1752 | 1909 | 1960-62 | 91I/L^{˜} | Q 213S | 256^{#} |
| | | | | | | A/C | | A/T | | | | | A/C | | A/T |
| | C ns | G | A | T | TTC | T T | C A A | G T | G | A | T | TTC | T T | C A A | G T |
| R | 1 | G | A | T | TTC | A T T | C A A | A G T | * | * | * | *** | * ** | * * * | * * * |
| R | 2 | G | A | T | TTC | A T T | C A A | A G T | * | * | * | *** | * ** | * * * | * * * |
| R | 7 | G | A | T | TTC | A T T | C A A | A G T | * | * | * | *** | * ** | * * * | * * * |
| R | 14 | G | A | T | TTC | A T T | C A A | A G T | * | * | * | *** | * ** | * * * | * * * |
| R | 18 | G | A | T | TTC | A T T | C A A | A G T | * | * | * | *** | * ** | * * * | * * * |
| R | 23 | G | A | T | TTC | A T T | C A A | A G T | * | * | * | *** | * ** | * * * | * * * |
| R | 25 | G | A | T | TTC | A T T | C A A | A G T | * | * | * | *** | * ** | * * * | * * * |
| NR | 3 | G | C | T | TTC | C T T | C A A | T G T | G | C | T | TTC | C T T | C A A | T G T |
| NR | 6 | G | A | T | TTC | A T T | C A A | A G T | G | A | T | TTC | A T T | C A A | A G T |
| NR | 9 | G | A | C | TAC | C T T | C A A | A G T | G | A | C | TAC | C T T | C A A | A G T |
| NR | 11 | G | C | T | TTC | C T T | C A A | T G T | G | C | T | TTC | C T T | C A A | T G T |
| NR | 15 | T | T | C | TGC | C T T | C A A | T G T | T | T | C | TGC | C T T | C A A | T G T |
| NR | 21 | G | A | C | GTC | A T T | T C G | A G T | G | A | C | GTC | A T T | T C G | A G T |
| NR | 22 | G | A | T | TTC | C T T | T C G | T G T | G | A | T | TTC | C T T | T C G | T G T |
| BK | 4 | G | A | T | TTC | C T T | C A A | T G T | T | T, A^{\hat{ }} | T | TTC | A T T | C A A | A G T |
| BK | 5 | G | A | T | TTC | C T T | C A A | T G T | G | A | T | TTC | C T T | C A A | A G T |
| BK | 8 | G | A | T | TAC | C T T | T C G | A G T | G | A | C | TAC | C T T | T C G | A G T |
| BK | 10 | G | T | C | TAA | C T T | T C G | T G T | G | A | C | TAA | C T T | T C G | T G T |
| BK | 12 | G | C | C | TAC | C T T | T C G | T G T | G | C | C | TAC | C T T | T C G | T G T |
| BK | 13 | G | A | T | TAC | A T T | C A A | A G T | G | C | T | TTC | C T T | C A A | A G T |
| BK | 16 | G | A | T | TTC | C T T | C A A | T G T | T | T | C | TGC | C T T | C A A | T G T |
| BK | 17 | T | T | C | TGC | C T T | C A A | T G T | T | T | C | TGC | C T T | C A A | T G T |
| BK | 19 | G | A | T | TTC | C T T | T C G | A G T | G | A | T | TTC | C T T | T C G | A G T |
| BK | 20 | G | C | T | TTC | C T T | T C G | T G T | G | A | C | TTC | C T T | T C G | T G T |
| BK | 24 | G | A | T | TTC | C T T | C A A | T G T | G | A | C | TTC | A T T | C A A | T G T |
| BK | 26 | G | C | C | TAA | C T T | C A A | T G T | G | C | C | TAA | C T T | C A A | T G T |

TABLE 18
Summary of HBV sequence markers absent in pretreatment serum samples of
sustained responders.

| | | | | | | | | | | | pre/core | | |
|---|---|---|---|---|---|---|---|---|---|---|---|---|---|
| P91 I/L | | | | | P256/S/C | | | pQ213S | | | promoter region | | |
| Outcome | No. | I | L | (p)# | S | C | (p)# | Q | S | (p)# | Wt.* | Mut* | (p)# |
| Responder | 7 | 7 | 0 | | 7 | 0 | | 7 | 0 | | 7 | 0 | |
| Non- | 7 | 2 | 5 | (<.3) | 3 | 4 | (ns.) | 5 | 2 | (ns.) | 2 | 5 | (<.03) |
| responder | | | | | | | | | | | | | |
| Breakthrough | 12 | 1^{˜} | 11 | (<.0003) | 3 | 9 | (<.004) | 7 | 5 | (ns.) | 7 | 5 | (ns.) |
| All Tx | 19 | 3 | 16 | (<.0003) | 6 | 13 | (<.006) | 12 | 7 | (ns.) | 9 | 10 | (<.03) |
| failures | | | | | | | | | | | | | |

The frequency of 4 sets of HBV sequence markers in pretreatment serum samples not observed in any of the patients exhibiting sustained response to lamivudine (minimum 27 months treatment), in the three response categories are listed. ‘all Tx failures’ designates a combination of patients in the non-response and breakthrough groups.

“#” indicates all significance listed is versus responder group (Fisher's 2-tailed exact t-test).

“n.s” indicates not significant (p<0.05)

“*” indicates frequency of the following group of mutations: G1739T, A1752C/T, T1909C, T1960G, T1961A/G, C1962A. Patients listed under mutations (‘mut.”) carried virus containing one or more of the 8 mutations listed in this group.

“a” indicates HBV in this patient converted from p91I (pretreatment) to p91L at breakthrough which occurred after 12 months of treatment

TABLE 19a
Polymerase additional sequence changes in Lamivudine treated patients

| | | | | Non | | Breakthrough | | | |
|---|---|---|---|---|---|---|---|---|---|
| | | Total | | Responder (7 pts) | | (12 pts) | | Responder (7 pts) | |
| A.ac Position | Nucl. Acid substit | Pre-treatm | Treatment | Pre-treatm | Treatment | Pre-treatm | Treatment | Pre-treatm | Treatment |
| 288-90* | Ser → Asn | 7 | 9 | 2 | 4 | 4 | 5 | 1 | NA |
| 291-93* | Thr → Tyr | 15 | 14 | 3 | 4 | 10 | 10 | 2 | NA |
| 330-32 | Gln → Pro | 1 | 9 | 0 | 0 | 0 | 0 | 1 | NA |
| 345-47 | Leu → Ile | 0 | 1 | 0 | 0 | 0 | 1 | 0 | NA |
| 357-59 | Asn → Asp | 3 | 2 | 1 | 1 | 1 | 1 | 1 | NA |
| 369-71 | Leu → Val, Ile | 0 | 3 | 0 | 1 | 0 | 2 | 0 | NA |
| 375-77 | Leu → Met | 0 | 1 | 0 | 1 | 0 | 0 | 0 | NA |
| 384-86 | Ser → Cys | 0 | 1 | 0 | 1 | 0 | 0 | 0 | NA |
| 417-19 | Ala → Ser | 3 | 3 | 1 | 1 | 2 | 2 | 0 | NA |
| 438-40 | Val → Ile | 4 | 2 | 1 | 1 | 0 | 1 | 3 | NA |
| 450-52 | Gly → Glu | 0 | 1 | 0 | 1 | 0 | 0 | 0 | NA |
| 459-61 | Arg → Gly | 4 | 3 | 1 | 1 | 1 | 2 | 2 | NA |
| 465-67 | Val → Ala | 1 | 1 | 1 | 1 | 0 | 0 | 0 | NA |
| 468-70 | Ala → Gly | 1 | 0 | 0 | 0 | 1 | 0 | 0 | NA |
| 474-76 | Leu → Val | 1 | 1 | 0 | 0 | 1 | 1 | 0 | NA |
| 480-82 | Ser → His, Cys | 2 | 1 | 1 | 1 | 0 | 0 | 1 | NA |
| 483-85 | Asn → Thr | 1 | 1 | 0 | 0 | 1 | 1 | 0 | NA |
| 495-97 | Ile → Leu, His, Phe, Asn | 26 | 19 | 7 | 7 | 12 | 12 | 7 | NA |
| 498-500 | Asn → Asp | 1 | 0 | 0 | 0 | 1 | 0 | 4 | NA |
| 507-09 | His → Tyr, Arg | 7 | 3 | 1 | 1 | 2 | 2 | 0 | NA |
| 513-15 | Thr → Ser, Ile | 1 | 1 | 0 | 0 | 1 | 1 | 2 | NA |
| 516-18 | Met → Leu | 2 | 0 | 0 | 0 | 0 | 0 | 3 | NA |
| 519-21 | Gln → Pro | 19 | 15 | 5 | 5 | 10 | 10 | 4 | NA |
| 522-24 | Asp → Asn | 25 | 18 | 7 | 6 | 12 | 12 | 6 | NA |
| 531-33 | Asp → Gly, Asn | 2 | 2 | 1 | 1 | 1 | 1 | 0 | NA |
| 534-36 | Ser → Tyr, Thr, His, | 5 | 4 | 2 | 2 | 2 | 2 | 1 | NA |
| | Asn | | | | | | | | |

TABLE 19b
Polymerase additional sequence changes in Lamivudine treated patients (cont'd)

| | | | | Non | | Breakthrough | | | |
|---|---|---|---|---|---|---|---|---|---|
| | | Total | | Responder (7 pts) | | (12 pts) | | Responder (7 pts) | |
| A.ac Position | Nucl. Acid substit | Pre-treatm | Treatment | Pre-treatm | Treatment | Pre-treatm | Treatment | Pre-treatm | Treatment |
| 543-45 | Arg → Asn, Lys | 3 | 0 | 2 | 2 | 1 | 1 | 0 | NA |
| 546-48 | Asn → Gin, Lys, His | 7 | 2 | 2 | 4 | 10 | 10 | 2 | NA |
| 552-54 | Tyr → His | 1 | 0 | 0 | 0 | 1 | 0 | 0 | NA |
| 555-57 | Val → Glu, Asp | 2 | 0 | 0 | 0 | 2 | 0 | 0 | NA |
| 564-66 | Leu → Met | 6 | 4 | 2 | 2 | 1 | 2 | 3 | NA |
| 576-78 | Lys → Gin | 21 | 17 | 6 | 5 | 11 | 12 | 4 | NA |
| 582-84 | Phe → Tyr | 3 | 3 | 1 | 2 | 0 | 1 | 2 | NA |
| 588-90 | Arg → Trp | 4 | 2 | 1 | 1 | 0 | 1 | 3 | NA |
| 618-20 | Ile → Val | 4 | 6 | 3 | 3 | 0 | 3 | 1 | NA |
| 621-23 | Leu → Met | 1 | 1 | 0 | 0 | 0 | 1 | 1 | NA |
| 690-02 | Ile → Leu | 1 | 0 | 0 | 0 | 1 | 0 | 0 | NA |
| 711-13 | Ala → Pro | 0 | 1 | 0 | 0 | 0 | 1 | 0 | NA |
| 756-58 | Leu → Ser, Phe | 1 | 2 | 0 | 1 | 0 | 1 | 1 | NA |
| 780-82 | Leu → Arg, Pro | 1 | 2 | 0 | 1 | 0 | 1 | 1 | NA |
| 783-85 | Glu → Arg | 0 | 1 | 0 | 1 | 0 | 0 | 0 | NA |
| 786-88 | Ser → Ala | 3 | 1 | 0 | 1 | 3 | 0 | 0 | NA |
| 792-94 | Phe → Tyr | 6 | 3 | 1 | 1 | 2 | 2 | 3 | NA |
| 843-45 | Asn → His, Ala, Asp, | 4 | 3 | 2 | 2 | 2 | 1 | 0 | NA |
| | Thr | | | | | | | | |
| 861-63 | Gly → Ala | 0 | 1 | 0 | 1 | 0 | 0 | 0 | NA |
| 873-75 | Asn → His, Gin | 5 | 6 | 3 | 2 | 2 | 4 | 0 | NA |
| 888-91 | Val → Ile | 2 | 1 | 1 | 1 | 0 | 0 | 1 | NA |
| 891-93 | Ile → Phe | 1 | 0 | 0 | 0 | 0 | 0 | 1 | NA |
| 894-96 | Gly → Ala | 0 | 1 | 0 | 1 | 0 | 0 | 0 | NA |
| 900-02* | Trp → Tyr, His | 19 | 17 | 5 | 5 | 10 | 12 | 4 | NA |
| 906-08* | Thr → Tyr, Ala | 17 | 15 | 5 | 5 | 11 | 10 | 1 | NA |

TABLE 20a
Precore/core additional sequence changes in Lamivudine treated patients

| | | | | Non | | Breakthrough | | | |
|---|---|---|---|---|---|---|---|---|---|
| | | Total | | Responder (7 pts) | | (12 pts) | | Responder (7 pts) | |
| A.ac Position | Nucl. Acid substit | Pre-treatm | Treatment | Pre-treatm | Treatment | Pre-treatm | Treatment | Pre-treatm | Treatment |
| 1672-74 | Ser → Phe | 1 | 0 | 1 | 0 | 0 | 0 | 0 | NA |
| 1675-77 | Gln → Leu | 19 | 15 | 6 | 6 | 11 | 9 | 2 | NA |
| 1678-80 | Gln → stop | 1 | 1 | 1 | 1 | 0 | 0 | 0 | NA |
| 1702-04 | His → Pro | 3 | 2 | 0 | 1 | 0 | 1 | 3 | NA |
| 1726-28 | Lys → Arg | 4 | 15 | 1 | 2 | 2 | 3 | 1 | NA |
| 1738-40 | Ser → Asn, Ile | 2 | 4 | 1 | 2 | 0 | 2 | 1 | NA |
| 1741-43 | Trp → Arg, stop | 2 | 2 | 1 | 1 | 1 | 1 | 0 | NA |
| 1753-55 | Leu → Ile | 1 | 2 | 0 | 0 | 1 | 2 | 0 | NA |
| 1756-58 | Gly → Asp | 16 | 15 | 5 | 5 | 10 | 9 | 3 | NA |
| 1759-61 | stop → Tyr | 1 | 1 | 1 | 1 | 0 | 0 | 0 | NA |
| 1765-67 | Ser → Phe | 1 | 1 | 0 | 1 | 0 | 0 | 1 | NA |
| 1768-70 | Leu → Met | 1 | 1 | 0 | 1 | 0 | 0 | 1 | NA |
| 1774-76 | stop → Ser | 1 | 3 | 0 | 0 | 1 | 3 | 0 | NA |
| 1792-94 | Asn → Lys | 1 | 0 | 0 | 0 | 0 | 0 | 1 | NA |
| 1801-03 | Val → Ala | 26 | 19 | 7 | 7 | 12 | 12 | 7 | NA |
| 1807-09 | Gln → His | 2 | 0 | 0 | 0 | 0 | 0 | 2 | NA |
| 1810-12 | His → Ser, Tyr, Ile | 2 | 4 | 0 | 1 | 1 | 3 | 1 | NA |
| 1816-18 | Ala → Val, Ser | 2 | 1 | 0 | 0 | 2 | 1 | 0 | NA |
| 1837-39 | Asn → Lys | 1 | 0 | 0 | 0 | 0 | 0 | 1 | NA |
| 1846-48 | Met → Leu | 26 | 19 | 7 | 7 | 12 | 12 | 7 | NA |
| 1849-51 | Phe → Tyr | 3 | 1 | 0 | 1 | 0 | 0 | 3 | NA |
| 1858-60 | Tyr → His | 3 | 1 | 0 | 0 | 0 | 1 | 3 | NA |
| 1861-63 | Cys → Phe | 2 | 1 | 0 | 1 | 0 | 0 | 2 | NA |

TABLE 20b
Precore/core additional sequence changes in Lamivudine treated patients (cont'd)

| | | | | Non | | Breakthrough | | | |
|---|---|---|---|---|---|---|---|---|---|
| | | Total | | Responder (7 pts) | | (12 pts) | | Responder (7 pts) | |
| A.ac Position | Nucl. Acid substit | Pre-treatm | Treatment | Pre-treatm | Treatment | Pre-treatm | Treatment | Pre-treatm | Treatment |
| 1903-05 | Gly → stop | 0 | 1 | 0 | 0 | 0 | 1 | 0 | NA |
| 1906-08 | His → Pro | 2 | 0 | 0 | 2 | 2 | 0 | 0 | NA |
| 1912-14 | Pro → Ser | 2 | 2 | 1 | 1 | 1 | 1 | 0 | NA |
| 1915-17 | Val → Leu | 24 | 18 | 7 | 11 | 11 | 11 | 7 | NA |
| 1918-20 | stop → Asp, Phe | 1 | 0 | 0 | 0 | 0 | 0 | 1 | NA |
| 1921-23 | Arg → Cys | 1 | 0 | 0 | 0 | 0 | 0 | 0 | NA |
| 1924-26 | Ile → Thr, Phe | 1 | 0 | 0 | 0 | 0 | 0 | 1 | NA |
| 1927-29 | Trp → Arg | 1 | 0 | 0 | 0 | 0 | 0 | 1 | NA |
| 1930-32 | Ser → Arg, Cys | 2 | 3 | 1 | 1 | 1 | 2 | 0 | NA |
| 1933-35 | Phe → Val, Cys, Tyr | 14 | 14 | 3 | 9 | 9 | 11 | 2 | NA |
| 1936-38 | Cys → Arg, Ser | 11 | 10 | 3 | 6 | 6 | 7 | 2 | NA |
| 1939-41 | Gly → Ala | 1 | 0 | 0 | 0 | 0 | 0 | 1 | NA |
| 1945-47 | Thr → Asn | 2 | 1 | 0 | 1 | 1 | 1 | 1 | NA |
| 1951-53 | Phe → Val, Ile | 17 | 14 | 2 | 10 | 10 | 10 | 5 | NA |
| 1963-65 | stop → Arg | 1 | 1 | 0 | 1 | 1 | 1 | 0 | NA |
| 1969-71 | Leu → Phe | 1 | 0 | 0 | 1 | 1 | 0 | 0 | NA |
| 1978-80* | Thr → Ser | 16 | 18 | 5 | 10 | 10 | 11 | 1 | NA |
| 1981-83* | Ser → Thr, Arg, Gln | 0 | 14 | 7 | 0 | 0 | 7 | 0 | NA |
| 1984-86* | Arg → Glu, Lys, Asp, | 0 | 10 | 0 | 0 | 0 | 7 | 0 | NA |
| | Asn | | | | | | | | |
| 1987-89* | Ser → Ile, Thr, Leu, Pro | 0 | 12 | 0 | 0 | 0 | 6 | 0 | NA |
| 1990-92* | Pro → Leu, Ser, Phe | 0 | 13 | 0 | 0 | 0 | 7 | 0 | NA |
| 1993-95* | Arg → Gln | 0 | 8 | 0 | 0 | 0 | 4 | 0 | NA |

TABLE 21
Polymerase and S gene nucleic acid substitutions

| Polymerase | Nucl. ac substit. | | S-surface | Nucl. a substit. | |
|---|---|---|---|---|---|
| 90 I | AUU | | 821 | AUA | |
| 91 L | CUU | | 821 | AUC | |
| L 180M | UUG | --> AUG | W172 | UGG | |
| M204I | AUG | --> AUU | W196 L | UGG | --> UUG |
| M204I | | --> AUC | W196 S | | --> UCG |
| M204V | AUG | --> GUG | I195M | AUA | --> AUG |
| S211T | UCU | --> ACU | S204 R | AGU | --> AGA |
| | | | S204 N | | --> AAU |
| | | | S204 K | | --> AAA |
| | | --> AAU | L205M | CUG | --> AUG |
| V212A | GUA | --> GCA | Y206 H | UAC | --> CAC |
| | | | Y206 H | | --> CAU |
| | | | Y206 S | | --> UCU |
| | | | Y206 L | | --> UUC |
| | | | Y206 C | | --> UGC |
| | | --> GGU | Y206 V | | --> GUU |
| Q213S | CAA | --> UCG | Y206 H | UAC | --> CAC |
| | | | Y206 H | | --> CAU |
| | | | Y206 S | | --> UCU |
| | | | Y206 L | | --> UUC |
| | | | Y206 C | | --> UGC |
| | | | Y206 V | | --> GUU |
| | | | N207 R | AAC | --> CGC |
| | | | N207 S | | --> AGC |
| 256 S | AGU | | outside | NA | |
| 256 C | UGU | | outside | NA | |

TABLE 22
Core and X gene nucleic acid substitutions

| Precore/core | Nucl. ac substit. | | X-region | Nucl. ac substit. | | Core | Nucl. ac substit | |
|---|---|---|---|---|---|---|---|---|
| S1738 I | AGU | --> AUU | E121 D (aa1736-8) | GAG | --> GAU | outside | NA | |
| S1738 M | AGU | --> AUG | L122 V (aa1739-40) | | | | | |
| R1750 S | AGU | --> AGC | E128 M (aa1749-51) | GAG | --> AUG | outside | NA | |
| | | --> AGU | I127L (aa17452-4) | AUU | --> CUU | | | |
| | | --> UGA | I127S (aa17452-4) | | --> UCU | | | |
| | | | I127N (aa17452-4) | | --> AAU | | | |
| | | | I127T (aa17452-4) | | --> ACU | | | |
| stop 1909 R | UGA | --> AGA | outside | NA | | I 3 (aa1907-9) | AUU | --> AUC |
| stop 1909 G | | --> GGU | | | | I 3 L (aa1907-9) | | --> CUU |
| | | | | | | I 3 V (aa1907-9) | | --> GUU |
| | | | | | | D 4 (aa1910-12) | GAU | --> GAC |
| P1960 Y | UCC | --> UAC | outside | NA | | P19 (aa1958-60) | CCU | --> CUG |
| P1960 C | | --> UGC | | | | S 20 T (aa1961-63) | UCU | --> ACU |
| P1960 stop | | --> UAA | | | | S 20 N (aa1961-63) | | --> AAU |
| P1960 V | | --> GUC | | | | S20 A (aa1961-63) | | --> GCU |

1. A method for predicting the long term response of a host of hepatitis B virus (HBV) to 3TC therapy comprising determining whether the HBV carried by the host (i) bears a nucleic acid that encodes for a leucine at amino acid position (aa) 91 in the DNA polymerase region (originally codon 438); or a (ii) a cysteine at aa256 (originally codon 604) in the DNA polymerase region of HBV.

2. A method for predicting the long term response of a host of hepatitis B virus (HBV) to 3TC therapy comprising determining whether the HBV carried by the host bears one or more of the following mutations: (i) Q213S (glutamine to serine at aa213) (originally codon 604) in the HBV polymerase region; (ii) G1739T, A1752C/F, T1909C, T1960G, or T1961A/G specific point mutation in the DNA precore/core promoter or open reading frame (ORF) region; or (iii) a pair of nucleotide changes A1738C and G1739T; A1750G and A1752G; T1909G and A1911T; or T1961A and C1962A representing specific double point mutations in the DNA precore/core promoter or open reading frame (ORF) region.

3. The method of claim 1 that determines whether the HBV carried by the host bears a nucleic acid that encodes for a leucine at amino acid position (aa) 91 in the DNA polymerase region.

4. The method of claim 1 that determines whether the HBV carried by the host bears a cysteine at aa256 (originally codon 604) in the DNA polymerase region of HBV.

5. The method of claim 2 that determines whether the HBV carried by the host bears mutation Q213S (glutamine to serine at aa213) in the HBV polymerase region.

6. The method of claim 2 that determines whether the HBV carried by the host bears mutation G1739T in the DNA precore/core promoter or open reading frame (ORF) region.

7. The method of claim 2 that determines whether the HBV carried by the host bears mutation A1752C/T in the DNA precore/core promoter or open reading frame (ORF) region.

8. The method of claim 2 that determines whether the HBV carried by the host bears mutation T1909C in the DNA precore/core promoter or open reading frame (ORF) region.

9. The method of claim 2 that determines whether the HBV carried by the host bears mutation T1960G in the DNA precore/core promoter or open reading frame (ORF) region.

10. The method of claim 2 that determines whether the HBV carried by the host bears mutation T1961A/G in the DNA precore/core promoter or open reading frame (ORF) region.

11. The method of claim 2 wherein the HBV bears a pair of nucleotide changes A1738C and G1739T representing specific double point mutations in the DNA precore/core promoter or open reading frame (ORF) region.

12. The method of claim 2 wherein the HBV bears a pair of nucleotide changes A1750G and A1752G representing specific double point mutations in the DNA precore/core promoter or open reading frame (ORF) region.

13. The method of claim 2 wherein the HBV bears a pair of nucleotide changes T1909G and A1911T representing specific double point mutations in the DNA precore/core promoter or open reading frame (ORF) region.

14. The method of claim 2 wherein the HBV bears a pari of nucleotide changes T1961A and C1962A representing specific double point mutations in the DNA precore/core promoter or open reading frame (ORF) region.

15. A method for determining the long term response of an HBV carrier to 3TC therapy, comprising determining whether a biological sample from the carrier contains an HBV protein, peptide, or peptide fragment that is encoded by a nucleic acid described in claim 1.

16. A method for determining the long term response of an HBV carrier to 3TC therapy, comprising determining whether a biological sample from the carrier contains an HBV protein, peptide, or peptide fragment that is encoded by a nucleic acid described in claim 2.

17. The method of claim 16, wherein the protein, peptide, or peptide fragment viral markers is detected by a method selected from the group consisting of western blot assay, two dimensional gel electrophoresis (2D-PAGE), enzyme linked immunosorbent assays (ELISA), enhanced chemiluminescence (ECL), immunohistochemistry, ELI-Spot assays, peptide sequencing, and antibody based protein array technology.

18. The method of claim 1, wherein the carrier is HBeAg negative.

19. The method of claim 2, wherein the carrier is HBeAg negative.

20. The method of claim 15, wherein the carrier is HBeAg negative.

21. The method of claim 16, wherein the carrier is HBeAg negative.

22. The method of claim 1 or 2, wherein the HBV nucleic acid sequence is determined by contact with a oligonucleotide probe having a sequence complementary to a section of the gene that includes the viral marker.

23. The method of claim 22, wherein probe is labeled probe using a detectable agent.

24. The method of claim 23, wherein the probe is labeled with a material selected from the grout consisting of a radioisotope, biotin, fluorescent dye, electron-dense reagent, enzyme, hapten or protein for which antibodies are available.

25. The method of claim 23, wherein the detectable label is assayed by spectroscopic, photochemical, biochemical, immunochemical, radioisotopic, or chemical means.

26. The method of claim 23, wherein the probe is detected by an oligomer restriction technique, a dot blot assay, a reverse dot blot assay, a line probe assay, or a 5′ nuclease assay.

27. The method of claim 23, wherein the probe is detected using DNA array technology.

28. The method of claim 27, wherein the probe is detected using a macroarray.

29. The method of claim 27, wherein the probe is detected using a microarray.

30. The method of claim 27, wherein the probe is detected using DNA microchip technology.

31. A nucleic acid sequence selected from the group consisting of Seq. Id Nos. 1-351 or an oligonucleotide which hybridizes under stringent conditions to a sequence selected from the group consisting of Seq. Id Nos. 1-351.

32. A nucleic acid sequence of 14 to 28 nucleotides illustrated in the embodiments of Tables 1-13 of Seq. Id Nos. 1-351 or an oligonucleotide which hybridizes under stringent conditions to a nucleic acid sequence of 14 to 28 nucleotides illustrated in the embodiments of Tables 1-13 of Seq. Id Nos. 1-351.

33. A kit for predicting the long term response of a host of hepatitis B virus (HBV) to 3TC therapy comprising reagents to determine whether the HBV carried by the host bears a nucleic acid that encodes for a leucine at amino acid position (aa) 91 in the DNA polymerase region (originally codon 438).

34. A kit for predicting the long term response of a host of hepatitis B virus (HBV) to 3TC therapy comprising reagents to determine whether the HBV carried by the host bears a cysteine at aa256 (originally codon 604) in the DNA polymerase region of HBV.

35. A kit for predicting the long term response of an HBV carrier to 3TC therapy, comprising reagents that can determine whether a biological sample from the carrier contains an HBV protein, peptide, or peptide fragment that is encoded by a nucleic acid described in claim 1.

36. A kit for predicting the long term response of an HBV carrier to 3TC therapy, comprising reagents that determining whether a biological sample from the carrier contains an HBV protein, peptide, or peptide fragment that is encoded by a nucleic acid described in claim 2.
